(19) United States
(12) Patent Application Publication
(10) Pub. No.: US 2012/0282180 A1
(43) Pub. Date: Nov. 8, 2012

(54) Compounds with Matrix-Metalloproteinase Inhibitory Activity and Imaging Agents Thereof

(76) Inventors: Hartmuth C. Kolb, Playa Del Rey, CA (US); Guenter Haufe, Muenster (DE); Malte Behrends, Lingen (DE); Klaus Kopka, Muenster (DE); Stefan Wagner, Muenster (DE); Verena Hugenberg, Bersenbrueck (DE); Hans-Joerg Breyholz, Muenster (DE); Sven Hermann, Muenster (DE); Michael Schaefers, Havixbeck (DE)

(21) Appl. No.: 13/155,558

(22) Filed: Jun. 8, 2011

(30) Foreign Application Priority Data
May 6, 2011 (EP) 11165157.6

(51) Int. Cl. A61K 51/04; A61K 31/216; C07D 213/55; A61K 31/44; A61P 35/00; A61K 31/18; C07D 309/10; A61K 31/351; A61P 9/00; A61P 29/00; C07C 311/16; A61K 31/195

(52) U.S. Cl. 424/1.89; 560/12; 514/538; 546/335; 514/357; 514/539; 562/430; 514/562; 564/89; 514/604; 549/419; 514/460; 424/1.85

(57) ABSTRACT

The present invention relates to the field of therapeutic and diagnostic agents and more specifically to compounds of formula (I) that are inhibitors of matrix-metalloproteinases (MMPs) and are useful in the treatment of diseases related thereto such as cardiovascular diseases, inflammatory diseases and malignant diseases. One embodiment of the invention is a compound of formula (I) labeled with a 18-fluorine atom having matrix metalloproteinase inhibitory activity suitable for diagnostic imaging. Also disclosed in the present invention is a pharmaceutical composition comprising the inhibitors of matrix-metalloproteinases (MMPs) of the invention or the corresponding labeled compounds useful as diagnostic imaging agents of the invention in a form suitable for mammalian administration. The invention furthermore discloses intermediates in the synthesis of the inhibitors of matrix-metalloproteinases (MMPs) of the invention and of the diagnostic imaging agents of the invention and kits for the preparation of the pharmaceutical composition of the invention.

### RELATED PATENT APPLICATIONS

This application claims priority to European patent application number EP 11 165 157.6, filed on May 6, 2011; the entire contents of which is incorporated by reference.

### TECHNICAL FIELD OF THE INVENTION

The present invention relates to the field of therapeutic and diagnostic agents and more specifically to compounds that are inhibitors of matrix-metalloproteinases (MMPs) and are useful in the treatment of diseases related thereto such as cardiovascular diseases, inflammatory diseases and malignant diseases. The compounds are also useful in the field of in vivo diagnostic imaging and in particular in (Positron Emission Tomography) PET imaging.

### DESCRIPTION OF RELATED ART

The matrix-metalloproteinases (MMPs) are a family of at least 20 zinc-dependent endo-peptidases which mediate degradation, or remodeling of the extracellular matrix (ECM) [Massova et al., FASEB J. 1998, 12, 1075-1095]. Together, the members of the MMP family can degrade e.g. components of the blood vessel wall and play a major role in both physiological and pathological events that involve the degradation of components of the ECM. Since the MMPs can interfere with the cell-matrix interactions that control cell behavior, their activity affects processes as diverse as cellular differentiation, migration, proliferation and apoptosis [Nagase and Woessner, J. Biol. Chem. 1999, 274, 21491-21494]. The negative regulatory controls that finely regulate MMP activity in physiological situations do not always function as they should. Inappropriate expression of MMP activity is thought to constitute part of the pathological mechanism in several disease states. MMPs are therefore targets for therapeutic inhibitors in many inflammatory, malignant and degenerative diseases [Whittaker et al., Chem. Rev. 1999, 99, 2735-2776].

Consequently, it is believed that synthetic inhibitors of MMPs may be useful in the treatment of many inflammatory, malignant and degenerative diseases. Furthermore, it has been suggested that inhibitors of MMPs may be useful in the diagnosis of these diseases. WO 01/60416 discloses compounds, which are proposed to be useful in the diagnosis of cardiovascular pathologies associated with extracellular matrix degradation such as atherosclerosis, heart failure and restenosis. The compounds disclosed therein comprise MMP inhibitors linked, via an optional linker, to a chelator capable of conjugating to a diagnostic metal. Preferred MMP inhibitors, chelators and linkers are described therein. A report by Zheng et al. [Nuc. Med. Biol. 2002, 29, 761-770] documented the synthesis of MMP inhibitors labeled with the positron emission tomography (PET) tracers ¹¹C and ¹⁸F. These compounds described therein are useful in the non-invasive imaging of breast cancer.

### SUMMARY OF THE INVENTION

Novel compounds having MMP inhibitory activity are disclosed, which have been found to be particularly useful in the prevention, treatment and diagnostic imaging of diseases associated with an unpaired activity of MMP, amongst others MMP-2, MMP-8, MMP-9 and/or MMP-13.

Another aspect of the present invention relates to pharmaceutical compositions useful in prevention, treatment and diagnostic imaging of diseases associated with an unpaired activity of MMP.

The compounds of the present invention are useful for the prevention, the treatment and the in vivo diagnostic imaging of a range of disease states (inflammatory, malignant and degenerative diseases) where specific matrix metalloproteinases are known to be involved. These include:

(a) atherosclerosis, where various MMPs are overexpressed. Elevated levels of MMP-1, 3, 7, 9, 11, 12, 13 and MT1-MMP have been detected in human atherosclerotic plaques [George, Exp. Opin. Invest. Drugs 2000, 9, 993-1007 and references therein]. Expression of MMP-2 [Li et al. Am. J. Pathol. 1996, 148, 121-128] and MMP-8 [Herman et al., Circulation 2001, 104, 1899-1904] in human atheroma has also been reported;
(b) CHF [Peterson et al., Matrix metalloproteinase inhibitor development for the treatment of heart failure, Drug Dev. Res. 2002, 55, 29-44] report that MMP-1, MMP-2, MMP-3, MMP-8, MMP-9, MMP-13 and MMP-14 are upregulated in heart failure;
(c) cancer [Vihinen et al., Int. J. Cancer 2002, 99, 157-186] reviews MMP involvement in cancers, and particularly highlights MMP-2, MMP-3, MMP-7, and MMP-9];
(d) arthritis [Meson et al., Inflamm. Res. 2001, 50, 183-186 “Selective matrix metalloproteinase inhibition in rheumatoid arthritis-targeting gelatinase A activation”], MMP-2 is particularly discussed;
(e) amyotrophic lateral sclerosis [Lim et al., J. Neurochem. 1996, 67, 251-259] where MMP-2 and MMP-9 are involved;
(f) brain metastases, where MMP-2, MMP-9 and MMP-13 have been reported to be implicated [Spinale, Circul. Res. 2002, 90, 520-530];
(g) cerebrovascular diseases, where MMP-2 and MMP-9 have been reported to be involved [Lukes et al., Mol. Neurobiol. 1999, 19, 267-284];
(h) Alzheimer's disease, where MMP-2 and MMP-9 have been identified in diseased tissue [Backstrom et al., J. Neurochem. 1992, 58, 983-992];
(i) neuroinflammatory diseases, where MMP-2, MMP-3 and MMP-9 are involved [Mun-Bryce et al., Brain. Res. 2002, 933, 42-49];
(j) COPD (i.e. chronic obstructive pulmonary disease) where MMP-1, MMP-2, MMP-8 and MMP-9 have been reported to be upregulated [Segura-Valdez et al., Chest. 2000, 117, 684-694];
(k) eye pathology [Kurpakus-Wheater et al., Prog. Histo. Cytochem. 2001, 36, 179-259];
(l) skin diseases [Herouy, Int. J. Mol. Med. 2001, 7, 3-12].

### DETAILED DESCRIPTION OF THE INVENTION

It has been surprisingly found that compounds of below general formula (I) wherein the group R³ is an alkenyl optionally substituted with a fluorine or an alkyl substituted with a fluorine act as inhibitors of MMP and in particular of MMP-2 and MMP-9. It has been surprisingly found that the presence of the group R³ as defined below in formula (I) provide these compounds with a very good inhibitory activity. Additionally, it has been surprisingly found that the inhibition is remarkable also in the (S)-enantiomer of the compounds of formula (I). This is particularly surprising because the (R)-enantiomer of the prior art analog compounds (e.g. hydroxamate derivatives such as CGS 27023A) are far more active than the corresponding (S)-enantiomers. For example, it is known that the (S)-enantiomer of compound CGS 27023A is far less potent then the corresponding (R)-enantiomer in the inhibition of MMP-2 and MMP-9 (see table 1). As a consequence, the use of the (S)-enantiomers or the racemates of the compounds of formula (I) as MMP inhibitors as well as the use of the (R)-enantiomers as MMP inhibitors is rendered possible by the introduction of the R³ group.

A first aspect of the present invention relates to compounds of formula (I)

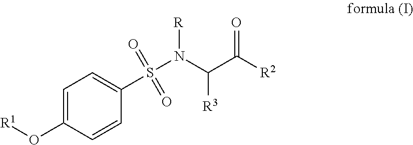

wherein
R is selected from the group of optionally substituted arylalkyl and optionally substituted heteroarylalkyl;
R¹ is C₁-C₆ alkyl, C₁-C₆ alkenyl, C₁-C₇ alkynyl wherein the C₁-C₆ alkyl, C₁-C₆ alkenyl, C₁-C₇ alkynyl are optionally substituted with one or more substituents selected from halogen, OH or OTs, or R¹ is (CH₂—CH₂—O—)ₙ—CH₂—CH₂—R⁵;
R² is C₁-C₆ alkyl, C₁-C₆ alkoxy or —NH—OR⁴;
R³ is C₁-C₆ fluoro-alkyl or optionally substituted C₂-C₆ alkenyl wherein the substituent is one or more F; preferably F is a fluorine atom on one of the carbons forming the double bond, preferably when R³ is a C₂-C₆ alkenyl substituted with F then R³ is a CH₂—C(F)═CH₂. Preferably, the F atom is on the carbon atom which is the second carbon atom of the alkyl or alkenyl chain of R³ counted starting from the first carbon atom of R³ attached to the rest of the molecule (e.g. —CH₂—C(F)═CH₂);
R⁴ is H, C₁-C₆ alkyl, C₃-C₆ cycloalkyl or C₅-C₆ heterocycloalkyl;
R⁵ is F, Cl, OH, OTs; and
   n is 0, 1, 2, 3, 4, 5 or 6, preferably n is 3.
   and enantiomers and pharmaceutically acceptable salts and ester thereof.

Preferably, in the compounds of formula (I)
R is pyrid-3-yl-CH₂— or phenyl-CH₂—;
R¹ is —CH₃, —CH₂—CH₂Cl, —CH₂—CH₂F, —CH₂—CH₂-OTs or —CH₂—CH₂OH;
R² is —NH—OR⁴;
R³ is —CH₂—CH₂F, —CH₂—C(F)═CH₂ or CH₂—CH═CH₂;
R⁴ is H, methyl, t-butyl or tetrahydropyranyl.

The pyrid-3-yl-CH₂— or the phenyl-CH₂— may be optionally substituted. Preferably, the substituent(s) when present is/are on the ring.

The compounds according to formula (I) have the carbon atom bearing radical R³ with a (S) configuration or the compounds according to formula (I) have the carbon atom bearing radical R³ with a (R) configuration.

In another aspect, the present invention relates to compounds of formula (I) wherein R denotes benzyl, 2-picolyl, and in particular 3-picolyl.

In another aspect, the present invention relates to compounds of formula (I) wherein R¹ is —CH₃, —CH₂—CH₂F, —CH₂—CH₂⁻¹⁸F, CH₂—CH₂—Cl, —CH₂—CH₂—OH or —CH₂—CH₂-OTs.

In another aspect, the present invention relates to compounds of formula (I) wherein R² is —NH—OH, —O-tert-butyl, or —OCH₃.

In another aspect, the present invention relates to compounds of formula (I) wherein R³ is selected from —CH₂—CH₂F, —CH₂—CH₂⁻¹⁸F, CH₂—C(F)═CH₂, —CH₂—C(¹⁸F)═CH₂ and CH₂—CH═CH₂.

In another aspect, the present invention relates to compounds wherein R⁴ is selected from H, tert-butyl and methyl.

In another aspect the present invention relates to compounds of formula (I) selected from:

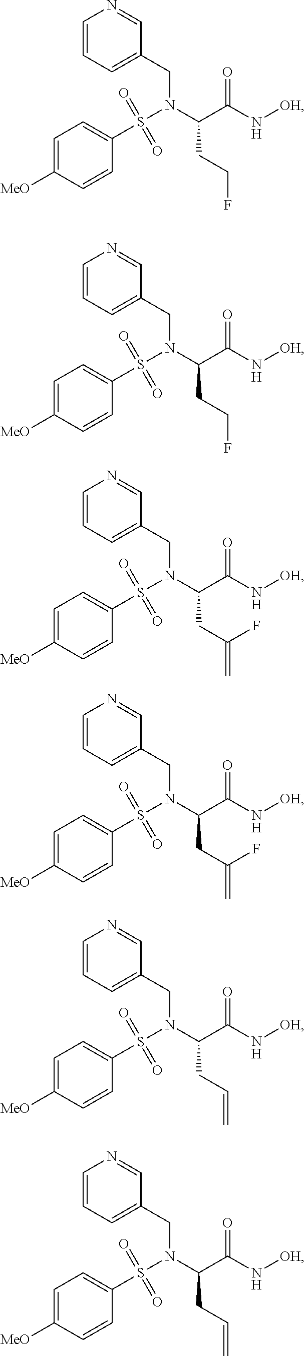

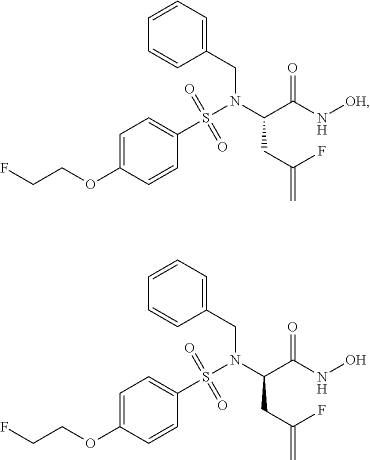

and the corresponding racemates, ester and pharmaceutically acceptable salts thereof.

In another aspect, the present invention relates to compounds of formula (I) which are substituted with ¹⁸F. It will be understood that other radionuclides may be used other than ¹⁸F. Such radionuclides may include radionuclides useful for PET imaging, which include ¹¹C, ¹³N, ¹⁵O, ¹⁸F, ⁶⁴Cu and ¹²⁴I, or those useful for SPECT imaging, such as ⁹⁹Tc, ⁷⁷Br, ⁶¹Cu, ¹⁵³Gd, ¹²³I, ¹²⁵I, ¹³¹I and ³²P.

In another aspect, the present invention relates to compounds of formula (I) wherein R³ is selected from C₁-C₆(¹⁸F) alkyl, C₂-C₆(¹⁸F) alkenyl. More preferably, R³ is CH₂CH₂—¹⁸F or —CH₂—C(¹⁸F)═CH₂.

In another aspect, the present invention relates to compounds of formula (I) wherein R¹ is C₁-C₆ alkyl substituted with a ¹⁸F. More preferably, R¹ is CH₂CH₂—¹⁸F.

In another aspect, the present invention relates to compounds of formula (I) wherein radical R is substituted with a ¹⁸F. Preferably, ¹⁸F is on the aryl or heteroaryl moiety of radical R or on a substituent of the aryl or heteroaryl moiety of radical R.

R⁵ in the compounds of formula (I) may be ¹⁸F.

Preferably, the compounds of formula (I) bear one ¹⁸F. In this case, ¹⁸F may be on a carbon atom of radical R or R¹ or R³, preferably as specified above or R⁵ can be ¹⁸F.

The compounds according to formula (I) as described above have the carbon atom bearing radical R³ with a (S) configuration or a (R) configuration.

In another aspect, the present invention relates to compounds of formula (I) for use as a medicament.

In another aspect, the present invention relates to compounds of formula (I) which are labeled with a ¹⁸F atom for use as a diagnostic agent, in particular as an in vivo diagnostic agent and more in particular in Positron Emission Tomography (PET).

In a further aspect, the present invention relates to compounds for use in the prevention and/or treatment of pathological conditions associated with unpaired expression of matrix-metalloproteases in human and animal, in particular mammals.

In another aspect, the present invention relates to compounds of formula (I) labeled with a ¹⁸F for use in the diagnosis of pathological conditions associated with unpaired expression of metalloproteases in human and animal, in particular mammals. In particular, the compounds are used in the in vivo diagnostic imaging, more in particular in PET.

The pathological conditions are selected from the group consisting of cardiovascular diseases, inflammatory diseases and malignant diseases. More in particular, the cardiovascular diseases are selected from atherosclerosis and congestive heart failure, the inflammatory disease is a chronic obstructive pulmonary disease, the malignant diseases are cancers.

The use of the diagnostic imaging compound of the invention permits the identification of active plaque burden, which allows risk stratification of patients with known or suspected coronary artery disease, i.e. patients with pain or a history of pain, or identified as high risk but asymptomatic. In addition, the diagnostic imaging agents of the invention permit identification of vulnerable plaques in symptomatic patients, which allows identification of high risk of acute myocardial infarction or stroke irrespective of stenosis and permits immediate risk stratification when the patient presents with chest pain. Furthermore, angioplasty of vulnerable plaques is high risk, and may lead to embolism of the artery tree post surgery. Thus imaging of this subtype of plaques may help reduce post-surgical complication.

In a further aspect the present invention relates to pharmaceutical compositions comprising a compound of formula (I).

Suitable preparations include for example tablets, capsules, suppositories, solutions, —particularly solutions for injection (s.c, i.v., i.m.) and infusion—syrups, elixirs, solution for inhalation.

The invention relates to pharmaceutical compositions comprising an effective amount, especially an amount effective in the treatment of one of the above-mentioned disorders, of the active ingredient together with pharmaceutically acceptable carriers that are suitable for topical, enteral, for example oral or rectal, or intravenous or parenteral administration and that may be inorganic or organic, solid or liquid. They are used for oral administration, especially tablets or gelatin capsules that comprise the active ingredient together with diluents, for example lactose, dextrose, sucrose, mannitol, sorbitol, cellulose and/or glycerol, and/or lubricants, for example silica, talc, stearic acid or salts thereof, such as magnesium or calcium stearate, and/or polyethylene glycol. Tablets may also comprise binders, for example magnesium aluminum silicate, starches, such as corn, wheat or rice starch, gelatin, methylcellulose, sodium carboxymethylcellulose and/or polyvinylpyrrolidone, and, if desired, disintegrators, for example starches, agar, alginic acid or a salt thereof, such as sodium alginate, and/or effervescent mixtures, or adsorbents, dyes, flavorings and sweeteners.

It is also possible to use the pharmacologically active compounds of the present invention in the form of intravenously and parentally administrable compositions or in the form of infusion solutions. Such solutions are preferably isotonic aqueous solutions or suspensions which, for example in the case of lyophilized compositions that comprise the active ingredient alone or together with a carrier, for example mannitol, can be made up prior to use. The pharmaceutical compositions may be sterilized and/or may comprise excipients, for example preservatives, stabilizers, wetting agents and/or emulsifiers, solubilizes, salts for regulating the osmotic pressure and/or buffers. The present pharmaceutical compositions are prepared in a manner known per se, for example by means of conventional mixing, granulating, confectioning, dissolving or lyophilizing processes, and comprise approximately from 1% to 95%, especially from approximately 1% to approximately 20%, active ingredient(s).

The present invention further provides a pharmaceutical composition which comprises a compound of formula (I) labeled with a ¹⁸F together with a biocompatible carrier, in a form suitable for mammalian, more in particular human administration.

In a further aspect, the present invention relates to a precursor useful in the preparation of the compounds of formula (I) labeled with a ¹⁸F atom. The precursor comprises a group suitable for reaction with ¹⁸F to give said compound of formula (I). Suitable precursors of the invention for preparation of imaging compounds of formula (I) are compounds of formula (I) which comprise a non-radioactive group (e.g. tosyl group) to permit ¹⁸F exchange, preferably via nucleophilic substitution reaction. Preferably, the radiofluorination with ¹⁸F-fluoride occurs via nucleophilic substitution reaction of a tosyl group on a precursor compound of the compounds of formula (I) with [¹⁸F] (Kryptofix222)KF.

Preferably the precursors are compounds of formula (II)

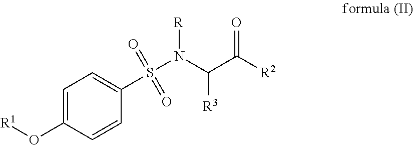

wherein R and R² are as defined above in formula (I); R¹ is an optionally substituted C₁-C₆ alkyl (preferably a C₂ alkyl), wherein the substituent is a OH, OTs or another leaving group for nucleophilic substitution; R³ is an optionally substituted C₁-C₆ alkyl or an optionally substituted C₂-C₆ alkenyl wherein the substituent is a OH, OTs or suitable leaving group for nucleophilic substitution, with the proviso that at least one of R¹ and R³ is substituted with a OH, OTs or another leaving group for nucleophilic substitution and/or with the proviso that when R¹ is substituted with a OH, OTs or another leaving group for nucleophilic substitution then R³ is C₁-C₆ fluoro alkyl or C₂-C₆ alkenyl optionally substituted with F. Preferably, in formula (II): R¹ is CH₂—CH₂-OTs and R³ is CH₂—CH₂F or CH₂—C(F)═CH₂ or CH₂—C(H)═CH₂; or R¹ is CH₃ and R³ is CH₂—CH₂OTs or CH₂—C(OTs)=CH₂.

In another aspect, the invention provides a process for preparing compounds of formula (I) having at least one carbon atom substituted with a ¹⁸F. The process comprises reacting a compound of formula (II)

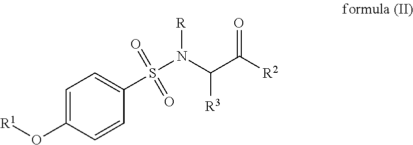

as defined above with a ¹⁸F containing reagent for substitution of a OH, OTs or another leaving group with ¹⁸F, preferably for substitution of OTs. Preferably, in formula (II):
R¹ is CH₂—CH₂-OTs and R³ is CH₂—CH₂—F or CH₂—C(F)═CH₂; or R¹ is CH₃ and R³ is CH₂—CH₂-OTs. Preferably, the ¹⁸F containing reagent is [¹⁸F] (Kryptofix222)KF.

In a further aspect, the present invention relates to compounds of formula (III) that are intermediate compounds in the synthesis of the compounds of the invention:

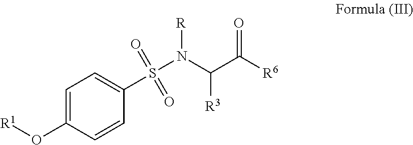

wherein
R, R¹ and R³ are as defined in formula (I) above; and
R⁶ is OH.

Compounds of formula (I) wherein R² is C₁-C₆ alkoxy can also be used as intermediate compounds in the synthesis of compounds of formula (I). In particular, they may be used in the synthesis of compounds of formula (I) wherein R² is NH—OR⁴ and R⁴ is H, C₁-C₆ alkyl, C₃-C₆ cycloalkyl or C₅-C₆ heterocycloalkyl.

Compounds of formula (I) wherein R² is NH—OR⁴ and R⁴ is C₁-C₆ alkyl, C₃-C₆ cycloalkyl or C₅-C₆ heterocycloalkyl can also be used as intermediate compound in the synthesis of compounds of formula (I). In particular, they are used in the synthesis of compounds of formula (I) wherein R² is NH—OR⁴ and R⁴ is H.

Intermediates according to the invention are for example compounds having the following structures:

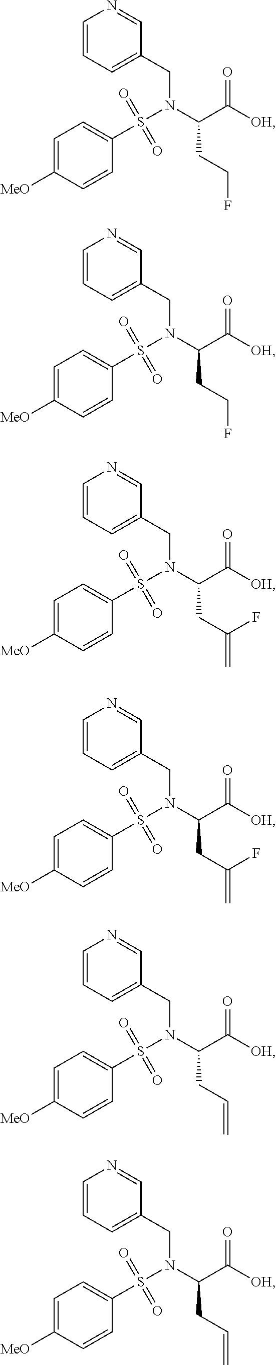

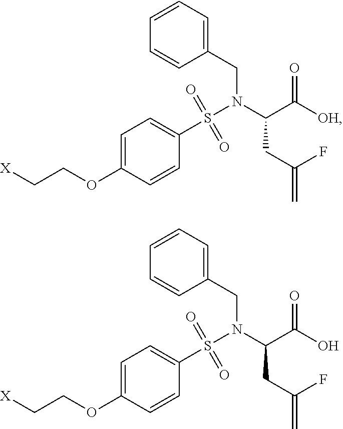

wherein X is selected from F, Cl, OH, OTs and ester thereof.

A further aspect of the present invention is a process for preparing a compound of formula (I)

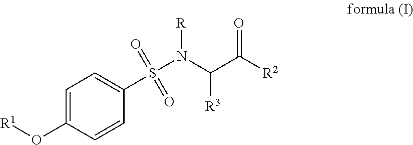

wherein
R, R¹ and R³ are as defined in formula (I) above; and
R² is NH—OR⁴; and
R⁴ is H, C₁-C₆ alkyl, C₃-C₆ cycloalkyl or C₅-C₆ heterocycloalkyl
   comprising
a) reacting a compound of formula (III′)

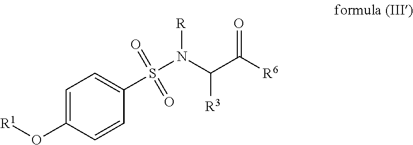

wherein
R, R¹ and R³ are as defined in formula (I)
and R⁶ is OH or C₁-C₆ alkoxy
   with an hydroxylamine derivative of formula (IV)

R⁴O—NH₂ formula (IV)

wherein R⁴ is as defined above.

A compound obtained in step a) having R⁴ selected from C₁-C₆ alkyl, C₃-C₆ cycloalkyl or C₅-C₆ heterocycloalkyl may be optionally hydrolyzed to a compound wherein R⁴ is H.

The compound of formula (III′) is prepared by
a′) reacting a compound of formula (V)

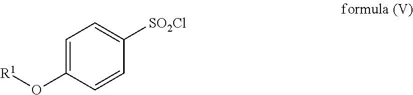

with a compound of formula (VI)

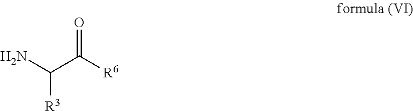

to obtain a compound of formula (VII)

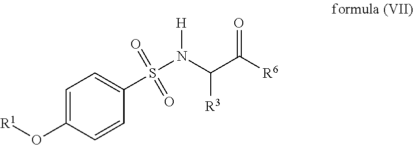

and
a″) reacting the compound of formula (VII) with a compound of formula (VIII)

R—Cl formula (VIII)
to obtain a compound of formula (III′)

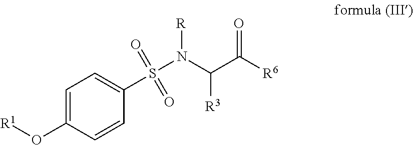

wherein
in any of the above formula (III′), (V), (VI), and (VII) and (VIII), R, R¹, R², R³, and R⁶ are as defined above.

Preferably, in step a′) pyridine is used as a solvent. Preferably, the reaction of step a″) is conducted in dimethylformamide (DMF) in the presence of potassium carbonate. Preferably, the reaction of step a) is conducted in the presence of dichloromethane, 1-Hydroxy-benzotriazol (HOB T), N-methyl-morpholin (NMM) and 1-(3-Dimethylaminopropyl)-3-ethylcarbodiimide (EDC) hydrochloride.

In a further aspect, the present invention provides a kit for the preparation of the pharmaceutical composition of the invention. Where the pharmaceutical composition of the invention comprises a diagnostic imaging agent labeled with a ¹⁸F, said kit comprises a precursor which is a compound of formula (I) comprising a group (e.g. tosyl group) suitable for reaction with a ¹⁸F containing compound (e.g. [¹⁸F] (Kryptofix222)KF) such that reaction of said precursor with said ¹⁸F containing compound gives said diagnostic imaging agent product of formula (I). Preferably said precursor is a compound of formula (II).

The kits may optionally further comprise additional components such as a radioprotectant, antimicrobial preservative, pH-adjusting agent or filler.

### DEFINITIONS

The term “C₁-C₆ alkyl”, when used either alone or within other terms such as “haloalkyl” and “arylalkyl” or “heteroarylalkyl” embraces linear or branched radicals having C₁-C₆ carbon atoms. Examples of such radicals include methyl, ethyl, n-propyl, isopropyl, n-butyl, isobutyl, tert-butyl, pentyl, isoamyl, hexyl. The term “alkylen” embraces bridging divalent alkyl radicals such as methylen and ethylen.

The term “C₂-C₆ alkenyl”, when used alone or in combination, embraces linear or branched radicals having at least one carbon-carbon double bond in a moiety having between two and six carbon atoms. Examples of alkenyl radicals include, without limitation, vinyl, propenyl, allyl, butenyl and 4-methylbutenyl. The term “alkenyl” encompasses radicals having “cis” and “trans” orientations, or alternatively, “E” and “Z” orientations, as appreciated by those of ordinary skill in the art.

The term “C₁-C₆ alkoxy” when used alone or in combination, embraces linear or branched oxygen-containing radicals each having alkyl portions of one to six carbon atoms. Examples of such radicals include methoxy, ethoxy, propoxy, butoxy and tert-butoxy. Alkoxy radicals may be further substituted with one or more halo atoms, such as fluoro, chloro or bromo, to provide “haloalkoxy” radicals. Examples of such radicals include fluoromethoxy, chloromethoxy, trifluoromethoxy, trifluoroethoxy, fluoroethoxy, fluoropropoxy and fluorobutoxy.

The term “halo”, when used alone or in combination, means halogens such as fluorine, chlorine, bromine or iodine atoms, preferably fluorine. The term “haloalkyl”, when used alone or in combination, embraces radicals wherein any one or more of the alkyl carbon atoms is substituted with halo as defined above. For example, this term includes monohaloalkyl, dihaloalkyl and polyhaloalkyl radicals such as a perhaloalkyl. A monohaloalkyl radical, for example, may have either an iodo, bromo, chloro or fluoro atom within the radical, preferably a fluoro atom. Dihalo and polyhaloalkyl radicals may have two or more of the same halo atoms or a combination of different halo radicals.

The term “C₁-C₆ haloalkyl” embraces radicals having 1-6 carbon atoms and, for example, haloalkyl radicals having one to three carbon atoms. Examples of haloalkyl radicals include fluoromethyl, difluoromethyl, trifluoromethyl, chloromethyl, dichloromethyl, trichloromethyl, pentafluoroethyl, heptafluoropropyl, chlorodifluoromethyl, dichlorofluoromethyl, fluoroethyl, difluoroethyl, trifluoroethyl, fluoropropyl, difluoropropyl, trifluoropropyl, chloroethyl, dichloroethyl and dichloropropyl.

The term “C₂-C₆ haloalkenyl”, when used alone or in combination, embraces radicals wherein anyone or more of the alkyl carbon atoms is substituted with halo as defined above, preferably a fluorine atom. For example, this term includes monohaloalkenyl, dihaloalkenyl and polyhaloalkenyl radicals. A monohaloalkenyl radical, for example, may have either an iodo, bromo, chloro or fluoro atom within the radical. Dihalo and polyhaloalkenyl radicals may have two or more of the same halo atoms or a combination of different halo radicals. Examples of haloalkenyl radicals include fluorovinyl, fluoropropenyl and fluorobutenyl.

The term “aryl”, when used alone or in combination, means a carbocyclic aromatic moiety containing one, two or even three rings wherein such rings may be attached together in a fused manner. Thus, the term “aryl” embraces aromatic radicals such as phenyl, naphthyl, indenyl, tetrahydronaphthyl, dihydrobenzofuranyl, anthracenyl, indanyl, benzodioxazinyl. The “aryl” group may be substituted, such as with 1 to 5 substituents including C₁-C₆ alkyl, hydroxyl, halo, halo-C₁-C₆-alkyl, nitro, cyano, C₁-C₆ alkoxy and C₁-C₆ alkylamino.

The term “heteroaryl”, as used herein, either alone or in combination, means a fully unsaturated (aromatic) ring moiety formed from carbon atoms and having one or more heteroatoms selected from nitrogen, oxygen and sulfur. The ring moiety or ring system may contain one (“monocyclic”), two (“bicyclic”) or even three (“tricyclic”) rings wherein such rings are attached together in a fused manner. Every ring of a “heteroaryl” ring system need not be aromatic, and the ring(s) fused thereto (to the heteroaromatic ring) may be partially or fully saturated and optionally include one or more heteroatoms selected from nitrogen, oxygen and sulfur.

Examples of unsaturated heteroaryl radicals, include unsaturated 5- to 6-membered heteromonocyclyl groups containing 1 to 4 nitrogen atoms, including for example, pyrrolyl, imidazolyl, pyrazolyl, 2-pyridyl, 3-pyridyl, 4-pyridyl, pyrimidyl, pyrazinyl, pyridazinyl and tetrazole; unsaturated 7- to 10-membered heterobicyclyl groups containing 1 to 4 nitrogen atoms, including for example, quinolinyl, isoquinolinyl, quinazolinyl, isoquinazolinyl, aza-quinazolinyl; unsaturated 5- to 6-membered heteromonocyclic groups containing an oxygen atom, for example, pyranyl, 2-furyl, 3-furyl, benzofuryl, etc.; unsaturated 5 to 6-membered heteromonocyclic groups containing a sulfur atom, for example, 2-thienyl, 3-thienyl, benzothienyl, etc.; unsaturated 5- to 6-membered heteromonocyclic groups containing 1 to 2 oxygen atoms and 1 to 3 nitrogen atoms, for example, oxazolyl, isoxazolyl, oxadiazolyl [e.g., 1,2,4-oxadiazolyl, 1,3,4-oxadiazolyl, 1,2,5-oxadiazolyl]; unsaturated 5 to 6-membered heteromonocyclic group containing 1 to 2 sulfur atoms and 1 to 3 nitrogen atoms, for example, thiazolyl, isothiazolyl, thiadiazolyl [e.g. 1,2,4-thiadiazolyl, 1,3,4-thiadiazolyl, 1,2,5-thiadiazolyl].

The term “aryl-alkyl” is any C₇-C₂₀ group consisting of an alkyl and an aryl group as defined above, preferably a benzyl group. Preferably the alkyl moiety of the “arylalkyl” group is a —CH₂- or a —CH₂—CH₂— group. Preferably the aryl moiety of the “arylalkyl” group is a phenyl. The “arylalkyl” may be optionally substituted with one or more substituents independently selected from OH, OTs, amino, halo in particular F and C₁, C₁-C₆ alkyl optionally substituted with OH, OTs, amino, halo in particular F and Cl or the “arylalkyl” may be optionally substituted with —O(CH₂—CH₂—O)ₙCH₂—CH₂—R⁵ wherein n is 1, 2, 3, 4, 5, 6, preferably 3, and R⁵ can be F, ¹⁸F, Cl, OH, OTs; or —(CH₂—CH₂—O)ₙCH₂—CH₂—R⁵ wherein n is 1, 2, 3, 4, 5, 6, preferably 3, and R⁵ can be F, ¹⁸F, Cl, OH, OTs. Preferably the substituent(s) are on the “aryl” moiety.

The term “heteroaryl-alkyl” is any C₇-C₂₀ group consisting of an alkyl and a heteroaryl group as defined above, preferably a picolyl group, more preferably a 3-picolyl group. Preferably, the alkyl moiety of the “heteroaryl-alkyl” group is a —CH₂— or a —CH₂—CH₂— group. Preferably, the heteroaryl moiety of the “heteroaryl-alkyl” group is a pyridyl, more preferably a 3-pyridyl. The “heteroaryl-alkyl” may be optionally substituted with one or more substituents independently selected from OH, OTs, amino, halo in particular F and C₁, C₁-C₆ alkyl optionally substituted with OH, OTs, amino, halo in particular F and Cl; the “heteroaryl-alkyl” may be optionally substituted with —O(CH₂—CH₂—O)ₙCH₂—CH₂—R⁵ wherein n is 1, 2, 3, 4, 5, 6, preferably 3, and R⁵ can be F, ¹⁸F, Cl, OH, OTs; or —(CH₂—CH₂—O)ₙCH₂—CH₂—R⁵ wherein n is 1, 2, 3, 4, 5, 6, preferably 3, and R⁵ can be F, ¹⁸F, Cl, OH, OTs. Preferably the substituent(s) are on the “aryl” moiety.

The term “C₅-C₆-heterocycloalkyl” relates to saturated or unsaturated mono ring comprising 5 or 6 carbon atoms, which carry heteroatoms such as N, O, S instead of one or more carbon atoms. Examples of such heterocyclic group are tetrahydrofuranyl, tetrahydropyranyl, pyrrolidinyl, pyrrolidino, piperidinyl, piperidino, piperazinyl, piperazino, morpholinyl, morpholino, thiomorpholinyl, thiomorpholino, tetrahydrothiofuranyl, and pyranyl

“Leavings group for nucleophilic substitution” are known to the skilled person in the art. They include e.g., Cl, Br, I etc.

The phrase “labeled with a ¹⁸F” used herein means that a compounds of the invention comprises a ¹⁸F either as an artificially enriched level of an atom intrinsic to the substructure, or as an additional essential feature that has been chemically attached via a functionality suitable for coupling said ¹⁸F.

By the term “biocompatible carrier” is meant a fluid, especially a liquid, in which the imaging agent can be suspended or dissolved, such that the composition is physiologically tolerable, i.e. it can be administered to the mammalian body without toxicity or undue discomfort. The biocompatible carrier is suitably an injectable carrier liquid such as sterile, pyrogen-free water for injection; an aqueous solution such as saline (which may advantageously be balanced so that the final product for injection is either isotonic or not hypotonic); an aqueous solution of one or more tonicity-adjusting substances (e.g. salts of plasma cations with biocompatible counter-ions), sugars (e.g. glucose or sucrose), sugar alcohols (e.g. sorbitol or mannitol), glycols (e.g. glycerol), or other non-ionic polyol materials (e.g. polyethyleneglycols, propylene glycols and the like).

By the term “biocompatible cation” is meant a positively charged counter-ion which forms a salt with an ionized, negatively charged group, where said positively charged counter-ion is also non-toxic and hence suitable for administration to the mammalian body, especially the human body. Examples of suitable biocompatible cations include the alkali metals sodium or potassium; the alkaline earth metals calcium and magnesium; and the ammonium ion. Preferred biocompatible cations are sodium and potassium, most preferably sodium.

By the term “radioprotectant” is meant a compound which inhibits degradation reactions, such as redox processes, by trapping highly-reactive free radicals, such as oxygen-containing free radicals arising from the radiolysis of water. The radioprotectants of the present invention are suitably chosen from: ascorbic acid, para-aminobenzoic acid (i.e. 4-aminobenzoic acid), gentisic acid (i.e. 2,5-dihydroxybenzoic acid) and salts thereof with a biocompatible cation as described above.

By the term “antimicrobial preservative” is meant an agent which inhibits the growth of potentially harmful micro-organisms such as bacteria, yeasts or moulds. The antimicrobial preservative may also exhibit some bactericidal properties, depending on the dose. The main role of the antimicrobial preservative(s) of the present invention is to inhibit the growth of any such micro-organism in the pharmaceutical composition post-reconstitution, i.e. in the radioactive diagnostic product itself. The antimicrobial preservative may, however, also optionally be used to inhibit the growth of potentially harmful microorganisms in one or more components of the kit of the present invention prior to reconstitution. Suitable antimicrobial preservatives include: the parabens, i.e. methyl, ethyl, propyl or butyl paraben or mixtures thereof; benzyl alcohol; phenol; cresol; cetrimide and thiomersal. Preferred antimicrobial preservative(s) are the parabens.

The term “pH-adjusting agent” means a compound or mixture of compounds useful to ensure that the pH of the reconstituted kit is within acceptable limits (approximately pH 4.0 to 10.5) for human or mammalian administration. Suitable such pH-adjusting agents include pharmaceutically acceptable buffers, such as tricine, phosphate or TRIS [i.e. tris(hydroxymethyl)aminomethane], and pharmaceutical acceptable bases such as sodium carbonate, sodium bicarbonate or mixtures thereof. When the ligand conjugate is employed in acid salt form, the pH-adjusting agent may optionally be provided in a separate vial or container, so that the user of the kit can adjust the pH as part of a multi-step procedure.

By the term “filler” is meant a pharmaceutically acceptable bulking agent which may facilitate material handling during production and lyophilisation. Suitable fillers include inorganic salts such as sodium chloride, and water soluble sugars or sugar alcohols such as sucrose, maltose, mannitol or trehalose.

### BRIEF DESCRIPTION OF THE FIGURES

FIG. 1 illustrates the synthetic route that was used to prepare precursor compounds bearing a fluorine atom on the moiety corresponding to radical R³ of formula (I) in the synthesis of the compounds of the invention. These syntheses were executed according to the following references:
D. M. Shendage, R. Fröhlich, K. Bergander, G. Haufe, Eur. J. Org. Chem. 2005, 719-727.
K.-W. Laue, S. Kröger, E. Wegelius, G. Haufe, Eur. J. Org. Chem. 2000, 3737-3743.
K.-W. Laue, C. Mück-Lichtenfeld, G. Haufe, Tetrahedron 1999, 55, 10413-10424.
K.-W. Laue, G. Haufe, Synthesis 1998, 1453-1456.
S. Kroger, G. Haufe, Amino Acids 1997, 12, 363-372.

FIG. 2a illustrates the synthetic route that was used to prepare compounds 1a, 1b, 3a, 3b, 4a, 4b, 5a and 5b, respectively.

FIG. 2b illustrates the synthetic route that was used to prepare compounds 6a, 6b, 8a, 8b, 9a, 9b, 10a and 10b, respectively.

FIG. 2c illustrates the synthetic route that was used to prepare compounds 16a, 16b, 17a, 17b, 18a, 18b, 19a and 19b, respectively.

FIG. 3 illustrates the synthetic route that was used to prepare compounds 20a, 20b, 21a, 21b, 22a, 22b, 23a and 23b, respectively.

FIGS. 4a-c illustrate the synthetic route that was used to prepare the compounds of formula (I) wherein R¹ is F—CH₂—CH₂, TsO—CH₂—CH₂, HO—CH₂—CH₂, Cl—CH₂—CH₂, respectively.

FIG. 5 illustrates the synthetic route that was used to prepare compounds 30a (MAB 254), 30b (MAB 255), 31a (MAB 258) and 31b (MAB 259).

FIG. 6 illustrates the synthetic route that was used to prepare e.g. compounds 33a (X═F), (HUG 78), and 33b (X═F), (HUG 74) or compound wherein X is OTs, OH, Cl.

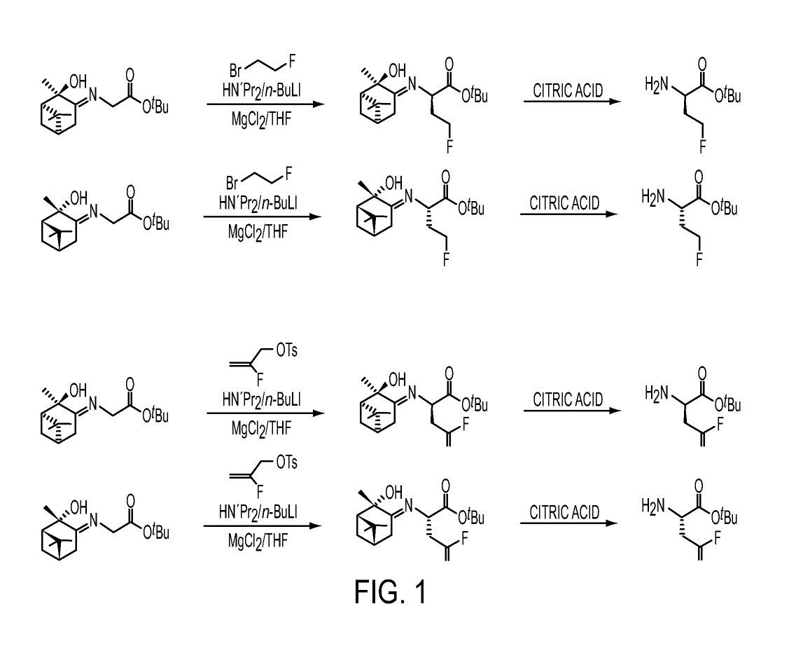

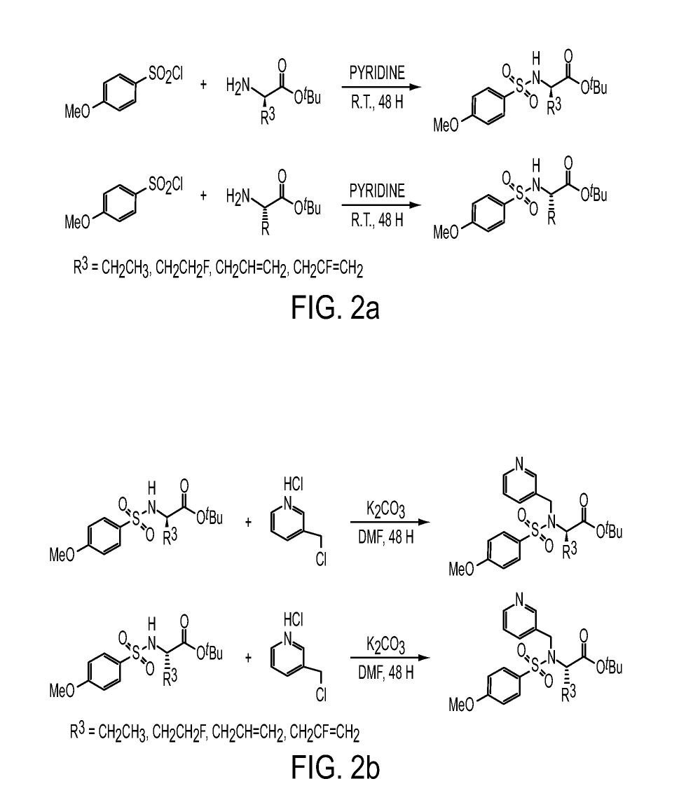

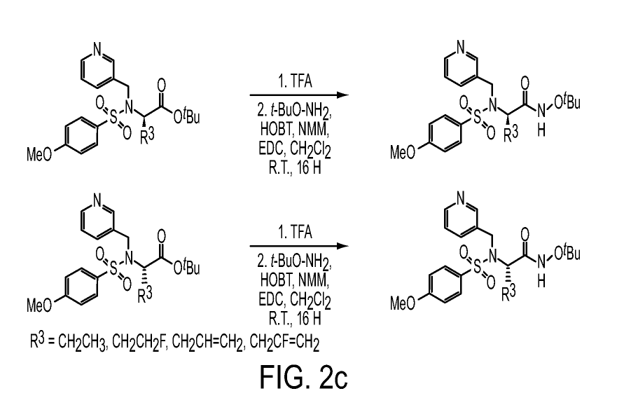

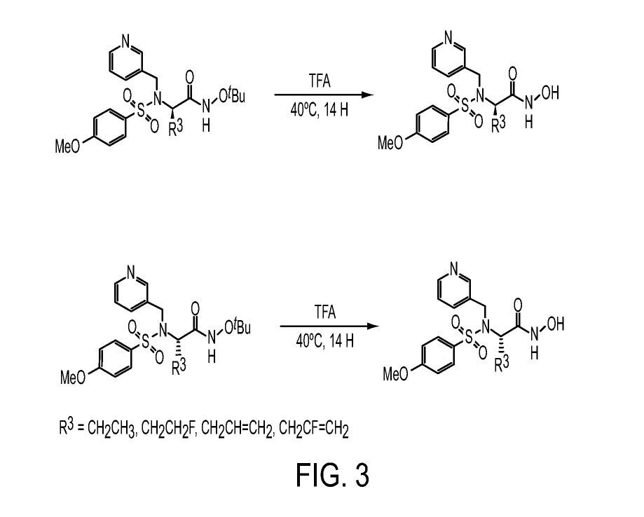

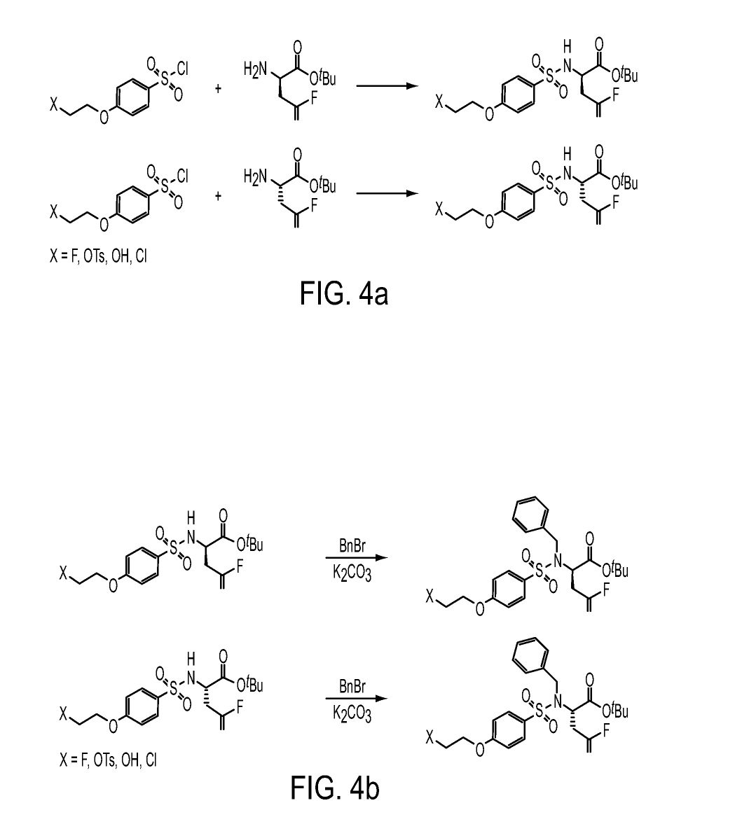

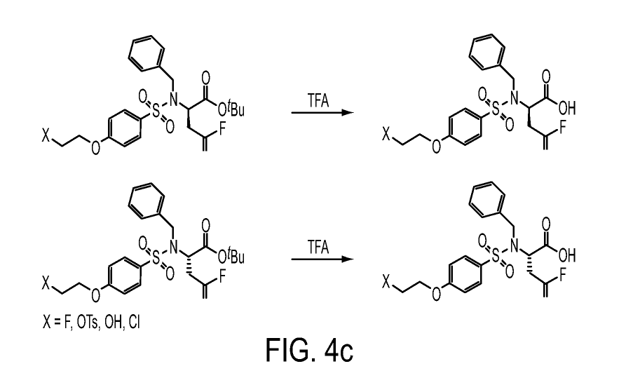

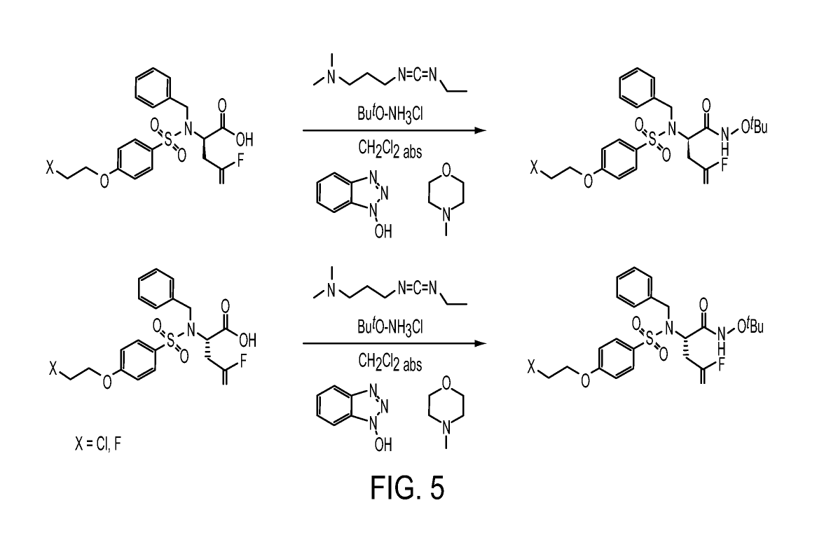

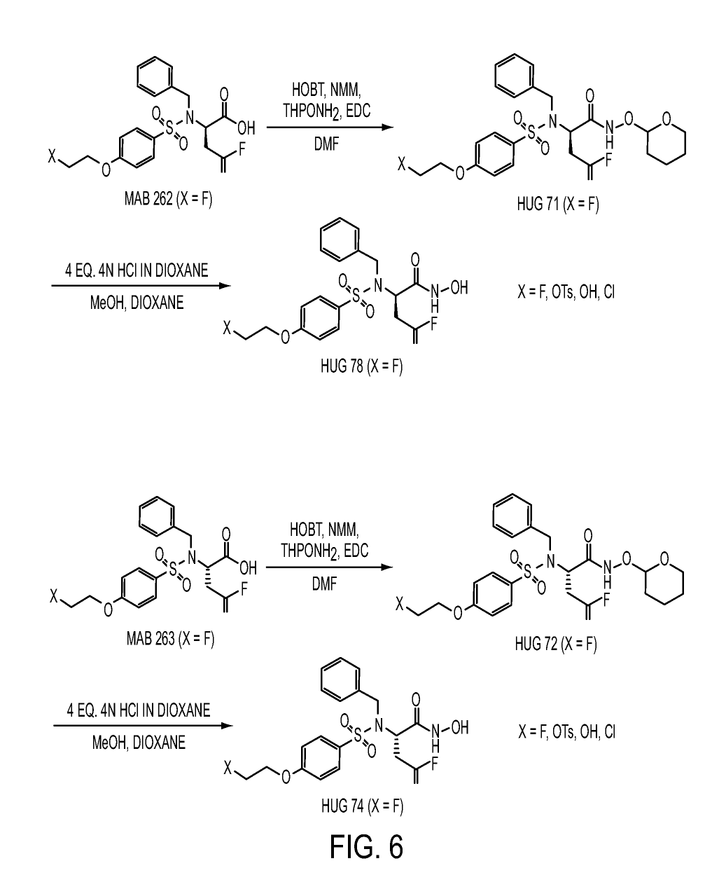

The syntheses disclosed in FIGS. 1 to 6 are useful for the preparation of the enantiomers of the compound of the invention. They are also useful in the preparation of the racemate of the compounds of the invention. In this case, the starting compounds of the respective synthesis are non-chiral molecules or they are in the form of racemate.

### EXAMPLES

### Example 1

### Example 1a

### Tert-butyl (S)—N-(4-methoxyphenylsulfonyl)aminobutanoate

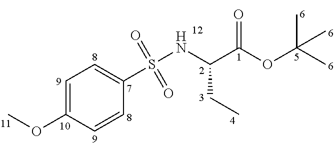

According to the general procedure tert-butyl (S)-aminobutanoate (630 mg, 3.96 mmol) in pyridin (15 mL) was treated with p-methoxyphenylsulfonyl chloride (818 mg, 3.96 mmol). The crude product was purified by column chromatography to get a white solid. Yield: 467 mg (36%). M.p. 82° C. ¹H NMR (300 MHz, CDCl₃): δ 0.92 (t, ³J_{H,H}=7.4 Hz, 3H, 4-CH₃), 1.27 (s, 9H, 6-CH₃), 1.61 (m, 1H, 3-CH_{A}), 1.77 (m, 1H, 3-CH_{B}), 3.72 (ddd, ³J_{H,H}=9.1 Hz, ³J_{H,H}=6.9 Hz, ³J_{H,H}=5.2 Hz, 1H, 2-CH), 3.85 (s, 3H, 11-CH₃), 5.19 (d, ³J_{H,H}=9.1 Hz, 1H, 12-NH), 6.95 (dm, ³J_{H,H}=9.0 Hz, 2H, 8-CH), 7.78 (dm, ³J_{H,H}=9.0 Hz, 2H, 9-CH). ¹³C NMR (75 MHz, CDCl₃): δ 9.2 (q, C-4), 26.8 (q, C-3), 27.7 (t, C-6), 55.6 (q, C-11), 57.1 (d, C-2), 82.2 (s, C-5), 114.1 (d, C-8), 129.4 (d, C-9), 131.4 (s, C-7), 162.9 (s, C-10), 170.8 (s, C-1). Exact mass (ESI⁺): C₁₅H₂₃NO₅S+Na⁺, calcd. 352.1195. found 352.1192, (C₁₅H₂₃NO₅S)₂+Na⁺, calcd. 681.2492. found 681.2483. MS (GC/MS, 70 eV): m/z (%)=329 (1) [M⁺], 228 (88) [M⁺—C₅H₉O₂], 171 (100) [M⁺—C₇H₇O₃S], 155 (5) [C₆H₅NO₂S⁺], 123 (18), 107 (21) [C₇H₇O⁺], 77 (13) [C₆H₅⁺], 57 (15) [C₄H₉⁺]. Optical rotation: [α]₅₈₉²⁰=+21.3, [α]₅₇₈²⁰=22.3, [α]₅₄₆²⁰=26.2, [α]₄₃₆²⁰=54.4, [α]₃₆₅²⁰=110.5 (c=1.009, CHCl₃).

### Example 1b

### Tert-butyl (R)—N-(4-methoxyphenylsulfonyl)aminobutanoate

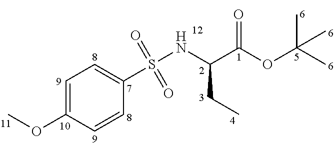

According to the general procedure tert-butyl (R)-aminobutanoic acid (551 mg, 3.46 mmol) was dissolved in pyridin (15 mL) and treated with p-methoxyphenylsulfonyl chloride (715 mg, 3.46 mmol). The crude product was purified by column chromatography to get a white solid. Yield: 446 mg (39%). M.p. 81° C. ¹H NMR (400 MHz, CDCl₃): δ 0.92 (t, ³J_{H,H}=7.4 Hz, 3H, 4-CH₃), 1.27 (s, 9H, 6-CH₃), 1.64 (m, 1H, 3-CH_{A}), 1.76 (ddd, ²J_{H,H}=14.7 Hz, ³J_{H,H}=7.4 Hz, ³J_{H,H}=5.2 Hz, 1H, 3-CH_{B}), 3.72 (ddd, ³J_{H,H}=9.1 Hz, ³J_{H,H}=7.0 Hz, ³J_{H,H}=5.2 Hz, 1H, 2-CH), 3.85 (s, 3H, 11-CH₃), 5.18 (d, ³J_{H,H}=9.1 Hz, 1H, 12-NH), 6.95 (dm, ³J_{H,H}=9.0 Hz, 2H, 8-CH), 7.78 (dm, ³J_{H,H}=9.0 Hz, 2H, 9-CH₃). ¹³C NMR (100 MHz, CDCl₃): δ 9.2 (q, C-4), 26.8 (t, C-3), 27.7 (q, C-6), 55.6 (q, C-11), 57.1 (d, C-2), 82.3 (s, C-5), 114.1 (d, C-8), 129.4 (d, C-9), 131.4 (s, C-7), 162.9 (s, C-10), 170.8 (s, C-1). Exact mass (ESI⁺): C₁₅H₂₃NO₅S+Na⁺, calcd. 352.1195. found 352.1189; (C₁₅H₂₃NO₅S)₂+Na⁺, calcd. 681.2492. found 681.2483. MS (GC/MS, 70 eV): m/z (%), 29 (3) [M⁺], 273 (1) [M⁺—C₄H₈], 228 (100) [M⁺—C₅H₉O₂], 171 (90) [C₇H₇O₃S⁺], 155 (4) [C₆H₅NO₂S⁺], 123 (22), 107 (29) [C₇H₇O⁺], 92 (10) [C₆H₄O⁺], 77 (15) [C₆H₅⁺], 57 (15) [C₄H₉⁺], 56 (48) [C₄H₈⁺]. Optical rotation: [α]₅₈₉²⁰=−20.6, [α]₅₇₈²⁰=−22.0, [α]₅₄₆²⁰=−26.8, [α]₄₃₆²⁰=−54.1, [α]₃₆₅²⁰=−109.9 (c=1.008, CHCl₃).

### Example 2

### Example 2a

### Methyl (S)—N-(4-methoxyphenylsulfonyl)aminobutanoate

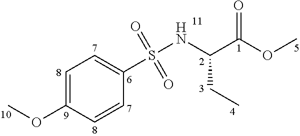

According to the general procedure 558 mg (3.63 mmol) methyl (S)-aminobutanoate hydrochloride was dissolved in pyridin (30 mL) and treated with p-methoxyphenylsulfonyl chloride (751 mg, 3.63 mmol). The crude product was purified by column chromatography to get a white solid. Yield: 450 mg (43%). M.p. 64° C. ¹H NMR (300 MHz, CDCl₃): δ 0.91 (t, ³J_{H,H}=7.4 Hz, 3H, 4-CH₃), 1.60-1.86 (m, 2H, 3-CH₂), 3.52 (s, 3H, 5-CH₃), 3.86 (s, 3H, 10-CH₃), 3.85 (m 1H, 2-CH), 5.30 (d, ³J_{H,H}=9.2 Hz, 1H, 11-NH), 6.93-6.99 (m, 2H, 7-CH), 7.75-7.81 (m, 2H, 8-CH). ¹³C NMR (75 MHz, CDCl₃): δ 9.3 (q, C-4), 26.6 (t, C-3), 52.4 (q, C-5), 55.6 (q, C-10), 56.7 (d, C-2), 114.0 (d, C-7), 129.3 (d, C-8), 131.2 (s, C-6), 162.9 (s, C-9), 172.1 (s, C-1). Elemental analysis: C₁₂H₁₇NO₅S (M=287.332 g/mol), calcd. C, 50.16; H, 5.96; N, 4.87. found C, 50.16; H, 5.91; N, 4.91%. Exact mass (ESI⁺): C₁₂H₁₇NO₅S+H⁺, calcd. 288.0906. found 288.0891. C₁₂H₁₇NO₅S+Na⁺, calcd. 310.0725. found 310.0718; (C₁₂H₁₇NO₅S)₂+Na⁺, 597.1553. found 597.1546. MS (GC/MS, 70 eV): m/z (%) 287 (5) [M⁺], 228 (70) [M⁺—CO₂CH₃], 171 (100) [C₇H₇O₃S⁺], 155 (5) [C₇H₇O₂S⁺], 123 (23) [C₇H₇O₂⁺], 107 (38) [C₇H₇O⁺], 92 (9), 77 (12) [C₆H₅⁺], 64 (6) [SO₂⁺], 59 (3) [CO₂CH₃⁺]. Optical Rotation: [α]₅₈₉²⁰=+8.4, [α]₅₇₈²⁰=+8.7, [α]₅₄₆²⁰=+10.7, [α]₄₃₆²⁰=+27.0, [α]₃₆₅²⁰=+64.0 (c=1.001, CHCl₃).

### Example 2b

### Methyl (R)—N-(4-methoxyphenylsulfonyl)aminobutanoate

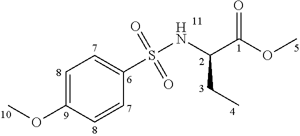

According to the general procedure methyl (R)-aminobutanoate hydrochloride (1000 mg, 6.51 mmol) was dissolved in pyridin (30 mL) and treated with p-methoxyphenylsulfonyl chloride (1350 mg, 6.51 mmol). The crude product was purified by column chromatography to get a white solid. Yield: 879 mg (47%). M.p. 63-64° C. ¹H NMR (300 MHz, CDCl₃): δ 0.91 (t, ³J_{H,H}=7.4 Hz, 3H, 4-CH₃), 1.60-1.84 (m, 2H, 3-CH₂), 3.52 (s, 3H, 5-CH₃), 3.86 (s, 3H, 10-CH₃), 3.85 (m 1H, 2-CH), 5.24 (d, ³J_{H,H}=9.2 Hz, 1H, 11-NH), 6.92-6.99 (m, 2H, 7-CH), 7.73-7.83 (m, 2H, 8-CH). ¹³C NMR (75 MHz, CDCl₃): δ 9.3 (q, C-4), 26.6 (t, C-3), 52.4 (q, C-5), 55.6 (q, C-10), 56.7 (d, C-2), 114.1 (d, C-7), 129.3 (d, C-8), 131.2 (s, C-6), 162.9 (s, C-9), 172.1 (s, C-1). Exact mass (ESI⁺): C₁₂H₁₇NO₅S+H⁺, calcd. 288.0906. found 288.0913; C₁₂H₁₇NO₅S+Na⁺, calcd. 310.0725. found 310.0733; (C₁₂H₁₇NO₅S)₂+Na⁺, calcd. 597.1553. found 597.1550. MS (ESI⁺, daughter ion experiment): m/z (%)=288 (9) [M⁺+H⁺], 228 (100) [M⁺−C₂H₃O₂], 171 (32) [C₇H₇O₃S⁺].

### Example 3

### Example 3a

### Tert-butyl (S)—N-(4-methoxyphenylsulfonyl)aminopent-4-enoate

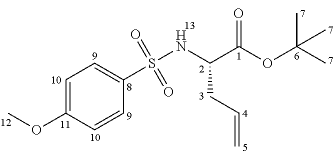

According to the general procedure tert-butyl (S)-aminopent-4-enoate (1200 mg, 7.02 mmol) was dissolved in pyridin (20 mL) and treated with p-methoxyphenylsulfonyl chloride (1454 mg, 7.02 mmol). The crude product was purified by column chromatography to get a white solid. Yield: 1150 mg (48%). M.p. 58-59° C. ¹H NMR (300 MHz, CDCl₃): δ 1.29 (s, 9H, 7-CH₃), 2.40-2.48 (m, 2H, 3-CH₂), 3.85 (s, 3H, 12-CH₃), 3.88 (dt, ³J_{H,H}=9.0 Hz, ³J_{H,H}=5.8 Hz, 1H, 13-NH), 5.05-5.13 (m, 2H, 5-CH₂), 5.20 (d, ³J_{H,H}=8.9 Hz, 1H, 2-CH), 5.66 (ddt, ³J_{H,H}=15.9 Hz, ³J_{H,H}=11.2 Hz, ³J_{H,H}=7.1 Hz, 1H, 4-CH), 6.95 (dm, ³J_{H,H}=9.0 Hz, 2H, 9-CH), 7.78 (dm, ³J_{H,H}=9.0 Hz, 2H, 10-CH). ¹³C NMR (75 MHz, CDCl₃): δ 27.8 (q, C-7), 37.9 (t, C-3), 55.5 (d, C-2), 55.6 (q, C-12), 82.6 (s, C-6), 114.2 (d, C-9), 119.3 (t, C-5), 129.4 (d, C-10), 131.6 (d, C-4 and C-8), 163.0 (s, C-11), 170.0 (s, C-1).

### Example 3b

### tert-Butyl (R)—N-(4-methoxyphenylsulfonyl)aminopent-4-enoate

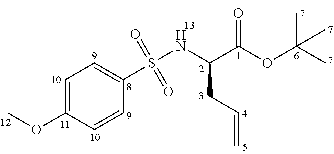

According to the general procedure tert-butyl (R)-aminopent-4-enoate (2140 mg, 12.48 mmol) was dissolved in pyridin (25 mL) and treated with p-methoxyphenylsulfonyl chloride (2570 mg, 12.48 mmol). The crude product was purified by column chromatography to get a white solid. Yield: 2375 mg (56%). M.p. 59° C. ¹H NMR (300 MHz, CDCl₃): δ 1.27 (s, 9H, 7-CH₃), 2.39-2.50 (m, 2H, 3-CH₂), 3.85 (s, 3H, 12-CH₃), 3.88 (dt, ³J_{H,H}=8.6 Hz, ³J_{H,H}=5.5 Hz, 1H, 13-NH), 5.04-5.13 (m, 2H, 5-CH₂), 5.20 (d, ³J_{H,H}=9.0 Hz, 1H, 2-CH), 5.67 (m, 1H, 4-CH), 6.95 (dm, ³J_{H,H}=9.0 Hz, 2H, 9-CH), 7.78 (dm, ³J_{H,H}=9.0 Hz, 2H, 10-CH). ¹³C NMR (75 MHz, CDCl₃): δ 27.7 (q, C-7), 37.9 (t, C-3), 55.4 (d, C-2), 55.6 (q, C-12), 82.5 (s, C-6), 114.1 (d, C-9), 119.4 (t, C-5), 129.4 (d, C-10), 131.4 (s, C-8), 131.5 (d, C-4), 162.9 (s, C-11), 169.9 (s, C-1). Exact mass (ESI⁺): C₁₆H₂₃NO₅S+Na⁺, calcd. 364.1195. found 364.1187; (C₁₆H₂₃NO₅S)₂+Na⁺, calcd. 705.2492. found 705.2488. MS (GC/MS, 70 eV): m/z (%) 364 (0) [M⁺], 300 (10) [M⁺-C₃H₅], 285 (4) [M⁺—C₄H₈], 240 (52) [M⁺—C₅H₉O₂], 171 (100) [C₇H₇O₃S⁺], 155 (5) [C₆H₅NO₂S⁺], 123 (36), 107 (50) [C₇H₇O⁺], 92 (13) [C₆H₄O⁺], 77 (24) [C₆H₅⁺], 64 (8) [SO₂⁺], 57 (42) [C₄H₉⁺], 56 (48) [C₄H₈⁺], 41 (40) [C₃H₅⁺]. Optical rotation: [α]₅₈₉²⁰=−17.1, [α]₅₇₈²⁰=−18.4, [α]₅₄₆²⁰=−20.4, [α]₄₃₆²⁰=−43.8, [α]₃₆₅²⁰=−88.5 (c=1.010, CHCl₃).

### Example 4

### Example 4a

### Tert-butyl (S)—N-(4-methoxyphenylsulfonyl)-γ-fluoro-α-aminobutanoate

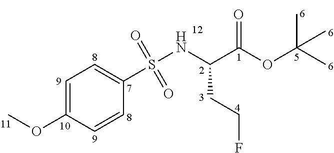

According to the general procedure tert-butyl (S)-2-amino-4-fluorobutanoate (1600 mg, 9.03 mmol) was dissolved in pyridin (15 mL) and treated with p-methoxyphenylsulfonyl chloride (1860 mg, 9.03 mmol). The crude product was purified by column chromatography to get a white solid. Yield: 2200 mg (70%). M.p. 89-90° C. ¹H NMR (300 MHz, CDCl₃): δ 1.28 (s, 9 H, 6-CH₃), 1.87-2.25 (m, ³J_{H,F}=26.2 Hz, ³J_{H,H}=5.5 Hz, 2H, 3-CH₂), 3.85 (s, 3H, 11-CH₃), 3.89 (m, ³J_{H,H}=8.0 Hz, 1H, 2-CH), 4.58 (dt, ²J_{H,F}=46.8 Hz, ³J_{H,H}=5.5 Hz, 2H, 4-CH₂F), 5.34 (d, ³J_{H,H}=8.0 Hz, 1H, 12-NH), 6.96 (dm, ³J_{H,H}=8.8 Hz, 2H, 8-CH), 7.79 (dm, ³J_{H,H}=8.8 Hz, 2H, 9-CH). ¹³C NMR (75 MHz, CDCl₃): δ 27.6 (q, C-6), 34.1 (dt, ²J_{H,F}=20.3 Hz, C-3), 52.7 (dd, ³J_{H,F}=3.7 Hz, C-2), 55.6 (q, C-11), 79.7 (dt, ¹J_{H,F}=165.9 Hz, C-4), 82.9 (s, C-5), 114.2 (d, C-8), 129.5 (d, C-9), 130.9 (s, C-7), 163.1 (s, C-10), 170.2 (s, C-1). ¹⁹F NMR (282 MHz, CDCl₃): δ −221.6 (tt, ²J_{H,F}=46.8 Hz, ³J_{H,F}=26.0 Hz, 4-CH₂F). Elemental analysis: C₁₅H₂₂FNO₅S (M=347.402 g/mol), calcd. C, 51.86; H, 6.38; N, 4.03. found C, 51.92; H, 6.24; N, 3.96%. Exact mass (ESI⁺): C₁₅H₂₂FNO₅S+Na⁺, cacld. 370.1100. found 370.1094. (C₁₅H₂₂FNO₅S)₂+Na⁺, calcd. 717.2303. found 717.2298. Optical rotation: [α]₅₈₉²⁰=−23.5, [α]₅₇₈²⁰=−25.1, [α]₅₄₆²⁰=−29.3, [α]₄₃₆²⁰=−58.4, [α]₃₆₅²⁰=−114.1 (c=1.021, CHCl₃).

### Example 4b

### Tert-butyl (R)—N-(4-methoxyphenylsulfonyl)-γ-fluoro-α-aminobutanoate

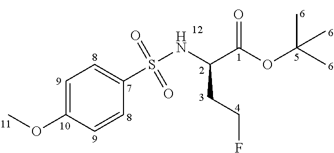

According to the general procedure tert-butyl (R)-2-amino-4-fluorobutanoate (1480 mg, 8.35 mmol) was dissolved in pyridin (15 mL) and treated with p-methoxyphenylsulfonyl chloride (1730 mg, 8.35 mmol). The crude product was purified by column chromatography to get a white solid. Yield: 1190 mg (41%). M.p. 89° C. ¹H NMR (400 MHz, CDCl₃): δ 1.28 (s, 9H, 6-CH₃), 1.90-2.23 (m, 2H, 3-CH₂), 3.85 (s, 3H, 11-CH₃), 3.89 (m, 1H, 12-NH), 4.58 (dt, ²J_{H,F}=46.8 Hz, ³J_{H,H}=5.5 Hz, 2H, 4-CH₂F), 5.38 (d, ³J_{H,H}=8.6, 1 H, 2-CH), 6.87-7.07 (m, 2H, 8-CH), 7.75-7.82 (m, 2H, 9-CH). ¹³C NMR (100 MHz, CDCl₃): δ 27.6 (q, C-6), 34.1 (dt, ²J_{C,F}=20.3 Hz, C-3), 52.7 (dt, ³J_{C,F}=3.7 Hz, C-2), 55.6 (q, C-11), 79.7 (dt, ¹J_{H,F}=165.9 Hz, C-4), 82.9 (s, C-5), 114.2 (s, C-8), 129.4 (d, C-9), 130.9 (s, C-7), 163.0 (s, C-10), 170.2 (s, C-1). ¹⁹F NMR (282 MHz, CDCl₃): δ −221.6 (tt, ²J_{H,F}=46.8 Hz, ³J_{H,F}=26.0 Hz, 4-CH₂F). Exact mass (ESI⁺): C₁₅H₂₂FNO₅S+Na⁺, calcd. 370.1100. found 370.1094. MS (GC/MS, 70 eV): m/z (%) 347 (2) [M⁺], 246 (82) [M⁺—CO₂C(CH₃)₃], 171 (100) [C₇H₇O₃S⁺], 155 (5) [177-CH₃], 123 (19) [C₆H₄OS⁺], 107 (38) [C₇H₇O⁺], 92 (18), 77 (27) [C₆H₅⁺], 64 (10) [SO₂⁺], 57 (52) [C₄H₉⁺]41 (35). Optical rotation: [α]₅₈₉²⁰=+24.7, [α]₅₇₈²⁰=+26.1, [α]₅₄₆²⁰=+30.4, [α]₄₃₆²⁰=+61.2, [α]₃₆₅²⁰=+118.4 (c=1.015, CHCl₃).

### Example 5

### Example 5a

### Tert-butyl (S)—N-(4-methoxyphenylsulfonyl)-γ-fluoro-α-aminopent-4-enoate

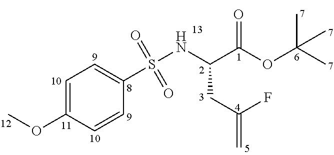

According to the general procedure tert-butyl (S)-2-amino-4-fluoropent-4-enoate (1360 mg, 7.19 mmol) was dissolved in pyridin (20 mL) and treated with p-methoxyphenylsulfonyl chloride (1490 mg, 7.19 mmol). The crude product was purified by column chromatography to get a greenish viscose liquid. Yield: 1.27 g (49%). ¹H NMR (300 MHz, CDCl₃): δ 1.30 (s, 9H, 7-CH₃), 2.58 (dd, ³J_{H,H}=5.7 Hz, ³J_{H,H}=3.6 Hz, 1H, 3-CH_{A}), 2.64 (d, ³J_{H,H}=5.9 Hz, 1 H, 3-CH_{B}), 3.85 (s, 3H, 12-CH₃), 3.99 (t, ³J_{H,H}=5.6 Hz, 1H, 2-CH), 4.34 (dd, ³J_{H,F}=49.3 Hz, ²J_{H,H}=3.0 Hz, 1H, 5-CHₜᵣₐₙₛ 4.63 (dd, ³J_{H,F}=17.1 Hz, ²J_{H,H}=3.0 Hz, 1H, 5-CH_{cis}), 5.41 (s, 1H, 13-NH), 6.96 (dm, ³J_{H,H}=9.0 Hz, 2H, 9-CH), 7.79 (dm, ³J_{H,H}=9.0 Hz, 2H, 10-CH). ¹³C NMR (75 MHz, CDCl₃): δ 27.6 (q, C-7), 36.4 (dt, ²J_{H,F}=27.5 Hz, C-3), 53.3 (d, C-2), 55.5 (q, C-12), 83.0 (s, C-6), 93.9 (dt, ²J_{H,F}=18.7 Hz, C-5), 114.1 (d, C-9), 129.4 (d, C-10), 131.3 (s, C-8), 161.0 (ds, ¹J_{H,F}=257.1 Hz, C-4), 163.0 (s, C-11), 169.2 (s, C-1). ¹⁹F NMR (282 MHz, CDCl₃): δ −96.0 (ddt, ³J_{H,F}=49.5 Hz, ³J_{H,F}=19.5 Hz, ³J_{H,F}=17.5 Hz, 1H). MS (ESI⁺, daughter ion experiment): m/z (%) 360 (0) [M⁺+H], 304 (8) [M⁺-C₄H₈], 284 (20) [304-F], 244 (10) [304-C₃H₄F], 214 (10) [M⁺-C₅H₉O₂—C₂H₂F], 188 (15) [M⁺-C₇H₇O₃S], 171 (100) [C₇H₇O₃S⁺], 107 (1) [C₇H₇O⁺], 88 (60), 77 (2) [C₆H₅⁺]. Exact mass (ESI⁺): C₁₆H₂₂FNO₅S+Na⁺, calcd. 382.1100. found 382.1096. Optical rotation: [α]₅₈₉²⁰=+3.0, [α]₅₇₈²⁰=+3.1, [α]₅₄₆²⁰=+4.1, [α]₄₃₆²⁰=+13.2, [α]₃₆₅²⁰=+36.8 (c=1.005, CHCl₃).

### Example 5b

### Tert-butyl (R)—N-(4-methoxyphenylsulfonyl)-γ-fluoro-α-aminopent-4-enoate

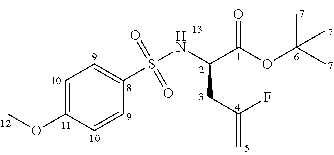

According to the general procedure tert-butyl (R)-2-amino-4-fluoro-pent-4-enoate (1362 mg, 7.20 mmol) was dissolved in pyridin (20 mL) and treated with p-methoxyphenylsulfonyl chloride (1490 mg, 7.20 mmol). The crude product was purified by column chromatography to get a greenish viscose liquid. Yield: 1.01 g (39%). ¹H NMR (400 MHz, CDCl₃): δ 1.30 (s, 9H, 7-CH₃), 2.45-2.69 (m, ³J_{H,F}=20.5 Hz, 2H, 3-CH₂), 3.85 (s, 3H, 12-CH₃), 3.98 (dt, ³J_{H,H}=8.9 Hz, ³J_{H,H}=5.8 Hz, 1H, 2-CH), 4.35 (dd, ³J_{H,F}=49.3, ²J_{H,H}=3.0 Hz, 1H, 5-CHₜᵣₐₙₛ), 4.64 (dd, ³J_{H,F}=17.1 Hz, ²J_{H,H}=3.0 Hz, 1H, 5-CH_{cis}), 5.33 (d, ³J_{H,H}=8.9 Hz, 1H, 13-NH), 6.96 (dm, ³J_{H,H}=9.0 Hz, 2H, 9-CH), 7.79 (dm, ³J_{H,H}=9.0 Hz, 2H, 10-CH). ¹³C NMR (100 MHz, CDCl₃): δ 27.6 (q, C-7), 36.5 (dt, ²J_{H,F}=27.5 Hz, C-3), 53.2 (d, C-2), 55.6 (q, C-12), 83.1 (s, C-6), 94.1 (dt, ²J_{H,F}=18.7 Hz, C-5), 114.2 (d, C-9), 129.4 (d, C-10), 131.2 (s, C-8), 161.0 (ds, ¹J_{H,F}=257.1 Hz, C-4), 163.0 (s, C-11), 169.2 (s, C-1). ¹⁹F NMR (282 MHz, CDCl₃): δ −96.1 (ddt, ³J_{H,F}=49.3 Hz, ³J_{H,F}=20.1 Hz, ³J_{H,F}=17.2 Hz, 4-CF). Elemental analysis: C₁₆H₂₂FNO₅S (M=359.413 g/mol), calcd. C, 53.47; H, 6.17; N, 3.90. found C, 53.44; H, 6.01; N, 3.83%. MS (GC/MS, 70 eV): m/z (%) 359 (0) [M⁺], 303 (7) [M⁺-C₄H₈], 300 (5) [M⁺—C₃H₄F], 258 (52) [M⁺-CO₂C(CH₃)₃], 244 (21) [258-CH₂], 187 (1) [C₇H₇O₃SNH₂⁺], 173 (9) [M⁺-C₇H₈O₃SNH/C₉H₁₄FO₂⁺], 171 (100) [C₇H₇O₃S⁺], 155 (8) [C₆H₅NO₂S⁺/C₉H₁₅O₂⁺], 123 (21), 107 (28) [C₇H₇O⁺], 92 (11), 78 (3) [NO₂S⁺], 77 (19) [C₆H₅⁺], 64 (7) [SO₂⁺], 59 (4) [C₃H₄F⁺], 57 (24) [C₄H₉⁺], 41 (6). Optical rotation: [α]₅₈₉²⁰=−0.8, [α]₅₇₈²⁰=−1.0, [α]₅₄₆²⁰=−1.2, [α]₄₃₆²⁰=−3.9, [α]₃₆₅²⁰=−10.2 (c=0.998, CHCl₃).

### Example 6

### Example 6a

### Tert-butyl (S)—N-(4-methoxyphenylsulfonyl)-N-(3-pyridylmethyl)-aminobutanoate

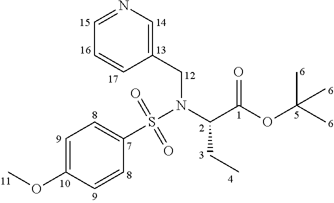

According to the general procedure tert-butyl (S)—N-(4-methoxyphenyl-sulfonyl)amino butanoate (450 mg, 1.37 mmol) was treated with potassium carbonate (1890 mg, 13.70 mmol) and picolyl chloride (225 mg, 1.37 mmol) in DMF (30 mL). After column chromatography (column Ø 3 cm×16 cm) the product was isolated as a white solid. Yield: 505 mg (88%). M.p. 170° C. ¹H NMR (400 MHz, CDCl₃): δ 0.80 (t, ³J_{H,H}=7.4 Hz, 3H, 4-CH₃), 1.31 (s, 9 H, 6-CH₃), 1.49 (ddd, ²J_{H,H}=14.1 Hz, ³J_{H,H}=11.5 Hz, ³J_{H,H}=6.1 Hz, 1H, 3-CH_{A}), 1.77 (m, 1H, 3-CH_{B}), 3.84 (s, 3H, 11-CH₃), 4.31 (dd, ³J_{H,H}=8.4 Hz, ³J_{H,H}=6.7 Hz, 1H, 2-CH), 4.50 (d, ²J_{H,H}=16.5 Hz, 1H, 12-CH_{A}), 4.71 (d, ²J_{H,H}=16.5 Hz, 1H, 12-CH_{B}), 6.94 (dm, ³J_{H,H}=9.0 Hz, 2H, 8-CH), 7.24 (dd, ³J_{H,H}=7.8 Hz, ³J_{H,H}=4.8 Hz, 1H, 16-CH), 7.72 (dm, ³J_{H,H}=8.9 Hz, 2H, 9-CH), 7.88 (dd, ³J_{H,H}=7.9 Hz, ⁴J_{H,H}=1.7 Hz, 1H, 17-CH), 8.49 (dd, ³J_{H,H}=4.8 Hz, ⁴J_{H,H}=1.5 Hz, 1H, 15-CH), 8.56 (d, ⁴J_{H,H}=1.9 Hz, 1H, 14-CH). ¹³C NMR (100 MHz, CDCl₃): δ 10.7 (q, C-4), 24.2 (t, C-3), 27.6 (q, C-6), 46.2 (t, C-12), 55.4 (q, C-11), 61.9 (d, C-2), 81.7 (s, C-5), 113.9 (d, C-8), 123.0 (d, C-16), 129.3 (d, C-9), 131.4 (s, C-7), 133.7 (s, C-13), 136.0 (d, C-17), 148.5 (d, C-15), 149.2 (d, C-14), 162.7 (s, C-10), 169.7 (s, C-1).

Optical rotation: [α]₅₈₉²⁰=−43.3, [α]₅₇₈²⁰=−45.5, [α]₅₄₆²⁰=−52.5, [α]₄₃₆²⁰=−96.2, [α]₃₆₅²⁰=−169.7 (c=1.013, CHCl₃):

### Example 6b

### Tert-butyl (R)—N-(4-methoxyphenylsulfonyl)-N-(3-pyridylmethyl)amino-butanoate

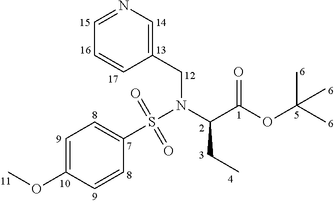

According to the general procedure tert-butyl (R)—N-(4-methoxyphenylsulfonyl)amino butanoate (338 mg, 1.03 mmol), potassium carbonate (1420 mg, 10.30 mmol) and picolyl chloride (169 mg, 1.03 mmol) in DMF (30 mL). After column chromatography (column Ø3 cm×16 cm) the product was isolated as a white solid. Yield: 394 mg (91%). M.p. 171° C. ¹H NMR (400 MHz, CDCl₃): δ 0.81 (t, ³J_{H,H}=7.4 Hz, 3H, 4-CH₃), 1.31 (s, 9H, 6-CH₃), 1.50 (m, ²J_{H,H}=14.5 Hz, 1H, 3-CH_{A}), 1.77 (m, ²J_{H,H}=14.5 Hz, ³J_{H,H}=7.3 Hz, 1H, 3-CH_{B}), 3.86 (s, 3H, 11-CH₃), 4.32 (dd, ³J_{H,H}=8.2 Hz, ³J_{H,H}=6.9 Hz, 1H, 2-CH), 4.51 (d, ³J_{H,H}=16.6 Hz, 1H, 12-CH_{A}), 4.71 (d, ³J_{H,H}=16.6 Hz, 1H, 12-CH_{B}), 6.94 (dm, ³J_{H,H}=9.0 Hz, 2H, 8-CH), 7.27 (dd, ³J_{H,H}=7.6 Hz, ³J_{H,H}=4.7 Hz, 1H, 16-CH), 7.72 (dm, ³J_{H,H}=9.0 Hz, 2H, 9-CH), 7.92 (dd, ³J_{H,H}=8.2 Hz, ⁴J_{H,H}=1.4 Hz, 1H, 17-CH), 8.51 (d, ³J_{H,H}=4.7 Hz, 1H, 15-CH), 8.55 (d, ⁴J_{H,H}=1.5 Hz, 1H, 14-CH). ¹³C NMR (100 MHz, CDCl₃). δ 11.0 (q, C-4), 24.5 (t, C-3), 27.8 (q, C-6), 46.4 (t, C-12), 55.6 (q, C-11), 62.1 (d, C-2), 82.9 (s, C-5), 114.1 (d, C-8), 123.4 (d, C-16), 129.5 (d, C-9), 131.6 (s, C-7), 134.1 (s, C-13), 136.6 (d, C-17), 148.5 (d, C-15), 149.0 (d, C-14), 162.9 (s, C-10), 167.0 (s, C-1). Exact mass (ESI⁺): C₂₁H₂₈N₂O₅S+H⁺, calcd. 421.1797. found 421.1788; C₂₁H₂₈N₂O₅S+Na⁺, calcd. 443.1617. found 443.1609; (C₂₁H₂₈N₂O₅S)₂+H⁺, calcd. 841.3516. found 841.3514; (C₂₁H₂₈N₂O₅S)₂+Na⁺, calcd. 863.3336. found 863.3341.

Optical rotation: [α]₅₈₉²⁰=+44.8, [α]₅₇₈²⁰=+47.0, [α]₅₄₆²⁰=+54.0, [α]₄₃₆²⁰=+98.7, [α]₃₆₅²⁰=+173.6 (c=1.011, CHCl₃).

### Example 7

### Example 7a

### Methyl (S)—N-(4-methoxyphenylsulfonyl)-N-(3-pyridylmethyl)amino-butanoate

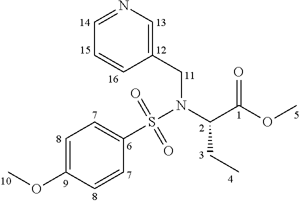

According to the general procedure methyl (S)—N-(4-methoxyphenylsulfonyl)-aminobutanoate (386 mg, 1.34 mmol) was treated with potassium carbonate (1850 mg, 12.40 mmol) and picolyl chloride (222 mg, 1.34 mmol) in DMF (15 mL). After column chromatography (column Ø2 cm×12 cm) the product was isolated as colorless highly viscose liquid. Yield: 384 mg (76%). ¹H NMR (300 MHz, CDCl₃). δ 0.80 (t, ³J_{H,H}=7.4 Hz, 3H, 4-CH₃), 1.54 (ddq, ²J_{H,H}=14.2 Hz, ³J_{H,H}=7.3 Hz, ³J_{H,H}=7.0 Hz, 1H, 3-CH_{A}), 1.81 (ddq, ²J_{H,H}=14.4 Hz, ³J_{H,H}=7.3 Hz, ³J_{H,H}=7.0 Hz, 1H, 3-CH_{B}), 3.43 (s, 3H, 5-CH₃), 3.86 (s, 3H, 10-CH₃), 4.43 (dd, ³J_{H,H}=8.1 Hz, ³J_{H,H}=7.0 Hz, 1H, 2-CH), 4.48 (d, ²J_{H,H}=16.5 Hz, 1H, 11-CH_{A}), 4.61 (d, ²J_{H,H}=16.4 Hz, 1H, 11-CH_{B}), 6.96 (d, ³J_{H,H}=9.0 Hz, 2H, 7-CH), 7.25 (dd, ³J_{H,H}=7.8 Hz, ³J_{H,H}=4.9 Hz, 1H, 15-CH), 7.72 (d, ³J_{H,H}=9.0 Hz, 2H, 8-CH), 7.84 (d, ³J_{H,H}=7.9 Hz, 1H, 16-CH), 8.50 (d, ³J_{H,H}=4.5 Hz, 1H, 14-CH), 8.54 (s, 1H, 13-CH). ¹³C NMR (75.48 MHz, CDCl₃): δ 10.8 (q, C-4), 23.9 (t, C-3), 46.4 (t, C-11), 51.9 (q, C-5), 55.6 (q, C-10), 61.2 (d, C-2), 114.0 (d, C-7), 123.3 (d, C-15), 129.5 (d, C-8), 131.2 (s, C-6), 133.5 (s, C-12), 136.4 (d, C-16), 148.7 (d, C-14), 149.1 (d, C-13), 163.0 (s, C-9), 171.2 (s, C-1). Exact mass (ESI⁺): C₁₈H₂₂N₂O₅S+H⁺, calcd. 379.1328. found 379.1325; C₁₈H₂₂N₂O₅S+Na⁺, calcd. 401.1147. found 401.1142; (C₁₈H₂₂N₂O₅S)₂+H², calcd. 757.2577. found 757.2583; (C₁₈H₂₂N₂O₅S)₂+Na⁺, calcd. 779.2397. found 779.2397. MS (GC-MS-EI): m/z (%) 378 (0) [M⁺], 349 (3) [M⁺-C₂H₅], 319 (100) [M⁺-CO₂CH₃], 207 (22) [M⁺-C₇H₇O₃S], 171 (24) [C₇H₇O₃S⁺], 147 (13) [C₅H₉NO₂S⁺], 123 (16) [C₇H₇O₂⁺], 107 (32) [C₇H₇O⁺], 92 (65) [C₆H₆N⁺], 77 (14) [C₆H₅⁺], 64 (5) [SO₂⁺], 59 (3) [CO₂CH₃⁺]. Optical rotation: [α]₅₈₉²⁰=−52.5, [α]₅₇₈²⁰=−55.0, [α]₅₄₆²⁰=−63.4, [α]₄₃₆²⁰=−115.8, [α]₃₆₅²⁰=−203.8 (c=1.010, CHCl₃).

### Example 7b

### Methyl (R)—N-(4-methoxyphenylsulfonyl)-N-(3-pyridylmethyl)amino-butanoate

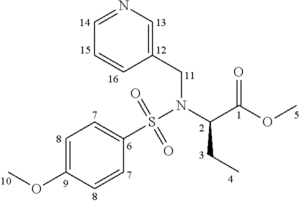

According to the general procedure methyl (R)—N-(4-methoxyphenylsulfonyl)-aminobutanoate (920 mg, 3.2 mmol), potassium carbonate (4770 mg, 32.0 mmol) and picolyl chloride (539 mg, 3.2 mmol) in DMF (15 mL). After column chromatography (column Ø 3 cm×19 cm) the product was isolated as a colorless highly viscose liquid. Yield: 1160 mg (96%). ¹H NMR (400 MHz, CDCl₃): δ 0.80 (t, ³J_{H,H}=7.4 Hz, 3H, 4-CH₃), 1.51 (m, ²J_{H,H}=21.5 Hz, ³J_{H,H}=7.3 Hz, 1H, 3-CH_{A}), 1.81 (dddd, ²J_{H,H}=21.5 Hz, ³J_{H,H}=14.4 Hz, ³J_{H,H}=7.3 Hz, ³J_{H,H}=7.0 Hz, 1H, 3-CH_{B}), 3.44 (s, 3H, 5-CH₃), 3.86 (s, 3H, 10-CH₃), 4.44 (dd, ³J_{H,H}=8.4 Hz, ³J_{H,H}=7.0 Hz, 1H, 2-CH), 4.48 (d, ²J_{H,H}=16.6 Hz, 1H, 11-CH_{A}), 4.61 (d, ²J_{H,H}=16.5 Hz, 1H, 11-CH_{B}), 6.96 (dm, ³J_{H,H}=9.0 Hz, 2H, 7-CH), 7.25 (ddd, ³J_{H,H}=7.9 Hz, ³J_{H,H}=4.8 Hz, ⁴J_{H,H}=0.5 Hz, 1H, 15-CH), 7.72 (dm, ³J_{H,H}=9.0 Hz, 2H, 8-CH), 7.85 (dt, ³J_{H,H}=7.9 Hz, ⁴J_{H,H}=1.9 Hz, 1H, 16-CH), 8.50 (dd, ³J_{H,H}=4.8 Hz, ⁴J_{H,H}=1.5 Hz, 1H, 14-CH), 8.53 (d, ⁴J_{H,H}=1.9 Hz, 1H, 13-CH). ¹³C NMR (100.62 MHz, CDCl₃): δ 10.6 (q, C-4), 23.7 (t, C-3), 46.3 (t, C-11), 51.7 (q, C-5), 55.4 (q, C-10), 61.1 (d, C-2), 113.8 (d, C-7), 123.1 (d, C-15), 129.3 (d, C-8), 131.0 (s, C-6), 133.2 (s, C-12), 136.1 (d, C-16), 148.6 (d, C-14), 149.1 (d, C-13), 162.8 (s, C-9), 170.9 (s, C-1). Exact mass (ESI⁺): C₁₈H₂₂N₂O₅S+H⁺, calcd. 379.1322. found 379.1314; C₁₈H₂₂N₂O₅S+Na⁺, calcd. 401.1142. found 401.1135; (C₁₈H₂₂N₂O₅S)₂+H⁺, cacld. 757.2572. found 757.2559; (C₁₈H₂₂N₂O₅S)₂+Na⁺, calcd 779.2391. found 779.2383. MS (ESI⁺, daughter ion experiment): m/z (%) 379 (85) [M⁺+H⁺], 319 (100) [M⁺-CO₂CH₃], 264 (8), 200 (30) [C₈H₁₀NO₃S⁺], 171 (22) [C₇H₇O₃S⁺], 123 (3) [C₇H₇O₂⁺].

### Example 8

### Example 8a

### Tert-butyl (S)—N-(4-methoxyphenylsulfonyl)-N-(3-pyridylmethyl)-2-amino-pent-4-enoate

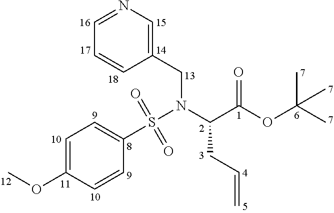

According to the general procedure tert-butyl (S)—N-(4-methoxyphenylsulfonyl)-aminopent-4-enoate (1084 mg, 3.17 mmol), potassium carbonate (4380 mg 31.70 mmol) and picolyl chloride (520 mg, 3.17 mmol) in DMF (25 mL). After column chromatography (column Ø 4 cm×16 cm) the product was isolated as a white solid. Yield: 750 mg (55%). M.p. 176-177° C. ¹H NMR (300 MHz, CDCl₃): δ 1.31 (s, 9H, 7-CH₃), 2.27 (ddd, ²J_{H,H}=14.7 Hz, ³J_{H,H}=8.0 Hz, ³J_{H,H}=6.9 Hz, 1H, 3-CH_{A}), 2.49 (dt, ³J_{H,H}=13.8 Hz, ³J_{H,H}=6.9 Hz, 1H, 3-CH_{B}), 3.86 (s, 3H, 12-CH₃), 4.49 (d, ²J_{H,H}=16.1 Hz, 1H, 13-CH_{A}), 4.51 (t, ³J_{H,H}=7.5 Hz, 1H, 2-CH), 4.69 (d, ²J_{H,H}=16.6 Hz, 1H, 13-CH_{B}), 4.89 (dd, ³J_{H,H}=17.1 Hz, ²J_{H,H}=1.5 Hz, 1H, 5-CHₜᵣₐₙₛ), 4.99 (dd, ³J_{H,H}=10.2 Hz, ²J_{H,H}=1.5 Hz, 1H, 5-CH_{cis}), 5.60 (ddt, ³J_{H,H}=17.0 Hz, ³J_{H,H}=10.3 Hz, ³J_{H,H}=6.7 Hz, 1H, 4-CH), 6.94 (dm, ³J_{H,H}=9.0 Hz, 2H, 9-CH), 7.25 (dd, ³J_{H,H}=7.9 Hz, ³J_{H,H}=4.9 Hz, 1H, 17-CH), 7.73 (dm, ³J_{H,H}=9.0 Hz, 2H, 10-CH), 7.86 (d, ³J_{H,H}=7.9 Hz, 1H, 18-CH), 8.50 (d, ³J_{H,H}=4.0 Hz, 1H, 16-CH), 8.54 (d, ⁴J_{H,H}=1.7 Hz, 1H, 15-CH). ¹³C NMR (75 MHz, CDCl₃): δ 27.8 (q, C-7), 35.5 (t, C-3), 46.6 (t, C-13), 55.6 (q, C-12), 60.2 (d, C-2), 82.3 (s, C-6), 114.1 (d, C-9), 118.4 (t, C-5), 123.3 (d, C-17), 129.6 (d, C-10), 131.5 (s, C-8), 133.0 (d, C-4), 133.7 (s, C-14), 136.6 (d, C-18), 148.5 (d, C-16), 149.0 (d, C-15), 163.0 (s, C-11), 169.3 (s, C-1). Exact mass (ESI⁺): C₂₂H₂₈N₂O₅S+H⁺, calcd. 433.1797. found 433.1812; C₂₂H₂₈N₂O₅S+Na⁺, calcd. 455.1617. found 455.1630, (C₂₂H₂₈N₂O₅S)₂₊H⁺, calcd. 865.3516. found 865.3532; (C₂₂H₂₈N₂O₅S)₂+Na⁺, calcd. 887.3336. found 887.3345. Optical rotation: [α]₅₈₉²⁰=−26.4, [α]₅₇₈²⁰=−27.3, [α]₅₄₆²⁰=−31.2, [α]₄₃₆²⁰=−56.6, [α]₃₆₅²⁰=−97.7 (c=1.001, CHCl₃):

### Example 8b

### Tert-butyl (R)—N-(4-methoxyphenylsulfonyl)-N-(3-pyridylmethyl)-2-aminopent-4-enoate

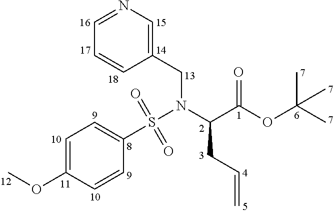

According to the general procedure tert-butyl (R)—N-(4-methoxyphenylsulfonyl)-aminopent-4-enoate (2375 mg, 6.96 mmol), was treated with potassium carbonate (9600 mg 69.60 mmol) and picolyl chloride (1140 mg 6.96 mmol) in DMF (45 mL). After column chromatography (column æ 5 cm×20 cm) the product was isolated as a white solid. Yield: 2010 mg (67%). M.p. 177° C. ¹H NMR (300 MHz, CDCl₃): δ 1.31 (s, 9H, 7-CH₃), 2.27 (m, 1H, 3-CH_{A}), 2.49 (m, 1H, 3-CH_{B}), 3.86 (s, 3H, 12-CH₃), 4.49 (d, ²J_{H,H}=16.4 Hz, 1H, 13-CH_{A}), 4.52 (t, ³, J_{H,H}=7.5 Hz, 1H, 2-CH), 4.69 (d, ²J_{H,H}=16.6 Hz, 1H, 13-CH_{B}), 4.89 (dq, ³J_{H,H}=17.1 Hz, ²J_{H,H}=1.4 Hz, 1H, 5-CH_{cis}), 4.99 (dd, ³J_{H,H}=10.3 Hz, ²J_{H,H}=1.4 Hz, 1H, 5-CHₜᵣₐₙₛ), 5.60 (ddt, ³J_{H,H}=17.0 Hz, ³J_{H,H}=10.3 Hz, ³J_{H,H}=6.7 Hz, 1H, 4-CH), 6.94 (dm, ³J_{H,H}=9.0 Hz, 2 H, 9-CH), 7.26 (ddd, ³J_{H,H}=4.8 Hz, ³J_{H,H}=3.1 Hz, ⁴J_{H,H}=0.6 Hz, 1H, 17-CH), 7.73 (dm, ³J_{H,H}=9.0 Hz, 2H, 10-CH), 7.87 (dt, ³J_{H,H}=7.9 Hz, ⁴J_{H,H}=1.8 Hz, 1H, 18-CH), 8.50 (dd, ³J_{H,H}=4.8 Hz, ⁴J_{H,H}=1.5 Hz, 1H, 16-CH), 8.54 (d, ⁴J_{H,H}=1.9 Hz, 1H, 15-CH). ¹³C NMR (75 MHz, CDCl₃): δ 27.8 (q, C-7), 35.5 (t, C-3), 46.6 (t, C-13), 55.6 (q, C-12), 60.2 (d, C-2), 82.3 (s, C-6), 114.1 (d, C-9), 118.4 (t, C-5), 123.3 (d, C-17), 129.6 (d, C-10), 131.5 (s, C-8), 133.0 (d, C-4), 133.7 (s, C-14), 136.7 (d, C-18), 148.4 (d, C-16), 149.0 (d, C-15), 163.0 (s, C-11), 169.3 (s, C-1). Exact mass (ESI⁺): C₂₂H₂₈N₂O₅S+H⁺, calcd. 433.1797. found 433.1782; C₂₂H₂₈N₂O₅S+Na⁺, calcd. 455.1617. found 455.1599; (C₂₂H₂₈N₂O₅S)₂+H⁺, calcd. 865.3516. found 865.3505; (C₂₂H₂₈N₂O₅S)₂+Na⁺, calcd. 887.3336. found 887.3318. MS (ESI⁺, daughter ion experiment): m/z (%) 433 (0) [M⁺+H⁺], 377 (12) [433-C₄H₈], 279 (4) [C₁₃H₁₅N₂O₃S⁺], 171 (15) [C₇H₇O₃S⁺], 165 (100) [C₈H₉N₂O₂⁺], 121 (24) [C₇H₉N₂⁺], 92 (18) [C₆H₆N⁺]. Optical rotation: [α]₅₈₉²⁰=+26.7, [α]₅₇₈²⁰=+27.8, [α]₅₄₆²⁰=+31.8, [α]₄₃₆²⁰=+57.2, [α]₃₆₅²⁰=+99.1 (c=1.009, CHCl₃).

### Example 9

### Example 9a

### Tert-butyl (S)—N-(4-methoxyphenylsulfonyl)-N-(3-pyridylmethyl)-2-amino-4-fluorobutanoate

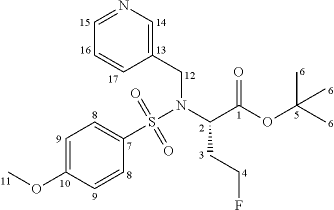

According to the general procedure tert-butyl (S)—N-(4-methoxyphenylsulfonyl)-2-amino-4-fluorobutanoate (1415 mg, 4.07 mmol), was reacted with potassium carbonate (5500 mg, 40.00 mmol) and picolyl chloride (673 mg 4.1 mmol) in DMF (20 mL). After column chromatographie (column Ø 4 cm×16 cm) the product was isolated as a greenish-white, viscose liquid. Yield: 1512 mg (85%). ¹H NMR (300 MHz, CDCl₃): δ 1.35 (s, 9H, 6-CH₃), 1.86 (m, 1H, 3-CH_{A}), 2.22 (ddt, ²J_{H,H}=13.6 Hz, ³J_{H,H}=9.7 Hz, ³J_{H,H}=6.0 Hz, 1H, 3-CH_{B}), 3.86 (s, 3H, 11-CH₃), 4.19-4.45 (m, 2H, 4-CH₂F), 4.36 (d, ²J_{H,H}=16.1 Hz, 1H, 12-CH_{A}), 4.49 (dd, ³J_{H,H}=7.7 Hz, ³J_{H,H}=6.6 Hz, 1H, 2-CH), 4.69 (d, ²J_{H,H}=16.2 Hz, 1H, 12-CH_{B}), 6.96 (dm, ³J_{H,H}=8.9 Hz, 2H, 8-CH), 7.25 (dd, ³J_{H,H}=7.9 Hz, ³J_{H,H}=4.8 Hz, 1H, 16-CH), 7.77 (dm, ³J_{H,H}=8.9 Hz, 2H, 9-CH), 7.82 (dm, ³J_{H,H}=8.4 Hz, 1H, 17-CH), 8.51 (s, 1H, 15-CH), 8.53 (s, 1H, 14-CH). ¹³C NMR (75 MHz, CDCl₃): δ 27.7 (q, C-6), 31.8 (dt, ²J_{H,F}=20.5 Hz, C-3), 47.3 (t, C-12), 55.6 (q, C-11), 57.0 (dd, ³J_{c}=4.3 Hz, C-2), 80.1 (dt, ¹J_{H,F}=166.3 Hz, C-4), 82.5 (s, C-5), 114.1 (d, C-8), 123.4 (d, C-16), 129.6 (d, C-9), 131.3 (s, C-7), 132.9 (s, C-13), 136.3 (d, C-17), 149.1 (d, C-15), 149.4 (d, C-14), 163.0 (s, C-10), 169.0 (s, C-1). ¹⁹F NMR (282 MHz, CDCl₃): δ −221.4 (ddt, ²J_{H,F}=46.9 Hz, ³J_{H,F}=28.3 Hz, ³J_{H,F}=22.8 Hz, 4-CH₂F). Exact mass (ESI⁺): C₂₁H₂₇N₂O₅S+H⁺, calcd. 439.1703. found 439.1701; C₂₁H₂₇N₂O₅S+Na⁺, calcd. 461.1522. found 431.1526; (C₂₁H₂₇N₂O₅S)₂+Na⁺, calcd. 877.3328. found 877.3320; (C₂₁H₂₇N₂O₅S)₂+Na⁺, calcd. 899.3147. found 899.3146. MS (ESI⁺, daughter ion experiment): m/z (%) 439 (15) [M⁺+H⁺], 383 (100) [439-C₄H₈], 171 (5) [C₇H₇O₃S⁺], 167 (10) [383-C₇H₇O₃S—CO₂H], 92 (3) [C₆H₆N⁺]. Optical rotation: [α]₅₈₉²⁰=+34.1, [α]₅₇₈²⁰=+35.5, [α]₅₄₆²⁰=+41.1, [α]₄₃₆²⁰=+74.5, [α]₃₆₅²⁰=n.d. (c=1.001, CHCl₃).

### Example 9b

### Tert-butyl (R)—N-(4-methoxyphenylsulfonyl)-N-(3-pyridylmethyl)-2-amino-4-fluorobutanoate

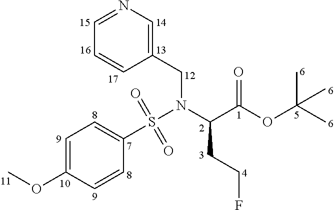

According to the general procedure tert-butyl (S)—N-(4-methoxyphenyl-sulfonyl)-2-amino-4-fluorobutanoate (500 mg, 1.44 mmol) was reacted with potassium carbonate (1990 mg 14.40 mmol) and picolyl chloride (237 mg, 1.44 mmol) in DMF (20 mL). After column chromatography (column Ø 2 cm×15 cm) the product was isolated as greenish-white, viscose liquid. Yield: 587 mg (93%). ¹H NMR (400 MHz, CDCl₃): δ 1.35 (s, 9H, 6-CH₃), 1.86 (m, ³J_{H,F}=28.3 Hz, ²J_{H,H}=14.2 Hz, 1H, 3-CH_{B}), 2.22 (dddt, ³J_{H,F}=22.7 Hz, ²J_{H,H}=14.2 Hz, ³J_{H,H}=7.5 Hz, ³J_{H,H}=6.6 Hz, 1H, 3-CH_{A}), 3.87 (s, 3H, 11-CH₃), 4.37 (d, ²J_{H,H}=16.2 Hz, 1H, 12-CH_{A}), 4.22-4.46 (m, ²J_{H,F}=47.1 Hz, 2H, 4-CH₂F), 4.48 (dd, ³J_{H,H}=7.9 Hz, ³J_{H,H}=6.7 Hz, 1H, 2-CH), 4.69 (d, ²J_{H,H}=16.2 Hz, 1H, 12-CH_{B}), 6.96 (dm, ³J_{H,H}=9.0 Hz, 2H, 8-CH), 7.25 (dd, ³J_{H,H}=7.9 Hz, ³J_{H,H}=4.8 Hz, 1H, 16-CH), 7.77 (dm, ³J_{H,H}=8.9 Hz, 2H, 9-CH), 7.82 (dt, ³J_{H,H}=7.9 Hz, ⁴J_{H,H}=1.8 Hz, 1H, 17-CH), 8.50 (d, ⁴J_{H,H}=2.0 Hz, 1H, 14-CH), 8.52 (dd, ³J_{H,H}=4.8 Hz, ⁴J_{H,H}=1.6 Hz, 1H, 15-CH). ¹³C NMR (100 MHz, CDCl₃): δ 27.8 (q, C-6), 31.9 (dt, ²J_{H,F}=20.5 Hz, C-3), 47.3 (t, C-12), 55.6 (q, C-11), 57.1 (dd, ³J_{H,F}=4.3 Hz, C-2), 80.2 (dt, ¹J_{H,F}=166.4 Hz, C-4), 82.6 (s, C-5), 114.2 (d, C-8), 123.4 (d, C-16), 129.7 (d, C-9), 131.4 (s, C-7), 132.9 (s, C-13), 136.3 (d, C-17), 149.2 (d, C-15), 149.5 (d, C-14), 163.1 (s, C-10), 169.1 (s, C-1). ¹⁹F NMR (282 MHz, CDCl₃): δ 221.4 (tdd, ²J_{H,F}=46.9 Hz, ³J_{H,F}=28.3 Hz, ³J_{H,F}=22.8 Hz, 4-CH₂F). Elemental analysis: C₂₁H₂₇FN₂O₅S (M=438.513 g/mol); calcd. C, 57.52; H, 6.21, N, 6.39. found C, 57.82; H, 6.00; N, 6.38%. Exact mass (ESI⁺): C₂₁H₂₇FN₂O₅S+H⁺, calcd. 439.1703. found 439.1697; C₂₁H₂₇FN₂O₅S+Na⁺, calcd. 461.1522. found 461.1513; (C₂₁H₂₇FN₂O₅S)₂+H⁺, calcd. 877.3328. found 877.3321; (C₂₁H₂₇FN₂O₅S)₂+Na⁺, calcd. 899.3147. found 899.3145. MS (GC/MS, 70 eV): m/z (%) 438 (0) [M⁺], 362 (1), 277 (3) [M⁺-C₈H₁₄FO₂], 191 (100), 171 (22) [C₇H₇O₃S⁺], 147 (75), 123 (12) [C₆H₄OS⁺], 107 (22) [C₇H₇O⁺], 92 (32) [C₆H₆N⁺], 77 (10) [C₆H₅⁺]. Optical rotation: [α]₅₈₉²⁰=−33.2, [α]₅₇₈²⁰=−34.5, [α]₅₄₆²⁰=−39.9, [α]₄₃₆²⁰=−72.6, [α]₃₆₅²⁰=−124.9 (c=1.001, CHCl₃).

### Example 10

### Example 10a

### Tert-butyl (S)—N-(4-methoxyphenylsulfonyl)-N-(3-pyridylmethyl)-2-amino-4-fluoropent-4-enoate

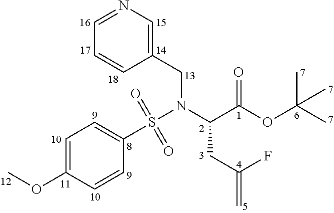

According to the general procedure tert-butyl (S)—N-(4-methoxyphenyl-sulfonyl)-2-amino-4-fluoropent-4-enoate (1500 mg, 4.17 mmol) was reacred with potassium carbonate (5760 mg 41.7 mmol) and picolyl chloride (685 mg, 4.17 mmol) in DMF (20 mL). After column chromatography (column Ø 4 cm×16 cm) the product was isolated as yellow, viscose liquid. Yield: 1428 mg (76%). ¹H NMR (300 MHz, CDCl₃): δ 1.35 (s, 9H, 7-CH₃), 2.48 (m, ³J_{H,F}=19.2 Hz, 1H, 3-CH_{A}), 2.73 (td, ³J_{H,F}=15.0 Hz, ³J_{H,H}=6.8 Hz, 1H, 3-CH_{B}), 3.86 (s, 3H, 12-CH₃), 4.14 (dd, ³J_{H,F}=49.7 Hz, ²J_{H,H}=3.1 Hz, 1H, 5-CHₜᵣₐₙₛ), 4.39 (d, ²J_{H,H}=16.3 Hz, 1H, 13-CH_{A}), 4.55 (dd, ³J_{H,F}=17.2 Hz, ²J_{H,H}=3.0 Hz, 1H, 5-CH_{cis}), 4.58 (t, ³J_{H,H}=7.3 Hz, 1H, 2-CH), 4.65 (d, ²J_{H,H}=16.3 Hz, 1H, 13-CH_{B}), 6.95 (d, ³J_{H,H}=9.0 Hz, 2H, 9-CH), 7.23 (ddd, ³J_{H,H}=7.9 Hz, ³J_{H,H}=4.8 Hz, ⁴J_{H,H}=0.7 Hz, 1H, 17-CH), 7.78 (m, 1H, 18-CH), 7.78 (d, ³J_{H,H}=9.0 Hz, 2H, 10-CH), 8.51 (dd, ³J_{H,H}=4.6 Hz, ⁴J_{H,H}=1.7 Hz, 2H, 15-CH and 16-CH). ¹³C NMR (75 MHz, CDCl₃): δ 27.8 (s, C-7), 34.0 (d, ²J_{H,F}=28.1 Hz, C-3), 47.4 (s, C-13), 55.6 (s, C-12), 57.6 (s, C-2), 82.8 (s, C-6), 93.4 (d, ²J_{H,F}=19.1 Hz, C-5), 114.2 (s, C-9), 123.3 (s, C-17), 129.8 (s, C-10), 131.4 (s, C-8), 132.8 (s, C-14), 136.4 (s, C-18), 149.1 (s, C-16), 149.5 (s, C-15), 161.5 (d, ¹J_{H,F}=256.5 Hz, C-4), 163.1 (s, C-11), 168.4 (s, C-1). ¹⁹F NMR (282 MHz, CDCl₃): δ −97.6 (dddd, ³J_{H,F}=50.0 Hz, ³J_{H,F}=19.2 Hz, ³J_{H,F}=17.2 Hz, ³J_{H,F}=15.0 Hz, 4-CF). Exact mass (ESI⁴): C₂₂H₂₇FN₂O₅S+Na⁺, cacld. 451.1703. found 451.1701; C₂₂H₂₇FN₂O₅S+Na⁺, calcd. 473.1522. found 473.1521; C₂₂H₂₇FN₂O₅S)₂+H⁺, calcd. 901.3328. found 901.3320; (C₂₂H₂₇FN₂O₅S)₂+Na⁺, calcd. 923.3147. found 923.3144.

Optical rotation: [α]₅₈₉²⁰=−6.3, [α]₅₇₈²⁰=−6.8, [α]₅₄₆²⁰=−7.9, [α]₄₃₆²⁰=−14.2, [α]₃₆₅²⁰=−22.9 (c=1.010, CHCl₃).

### Example 10b

### Tert-butyl (R)—N-(4-methoxyphenylsulfonyl)-N-(3-pyridylmethyl)-2-amino-4-fluoropent-4-enoate

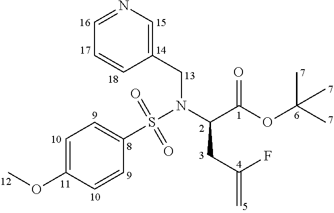

According to the general procedure tert-butyl (R)—N-(4-methoxyphenyl-sulfonyl)-2-amino-4-fluoropent-4-enoate (720 mg, 2.00 mmol) was reacted with potassium carbonate (2760 mg, 20.00 mmol) and picolyl chloride (329 mg, 2.00 mmol) in DMF (20 mL). After column chromatography the product was isolated as yellow, viscose oil. Yield: 523 g (58%). ¹H NMR (500 MHz, CDCl₃): δ 1.37 (s, 9H, 7-CH₃), 2.48 (ddd, ³J_{H,F}=19.2 Hz, ²J_{H,H}=15.1 Hz, ³J_{H,H}=7.8 Hz, 1H, 3-CH_{A}), 2.74 (dt, ²J_{H,H}=15.2 Hz, ³J_{H,H}=6.9 Hz, 1H, 3-CH_{B}), 3.88 (s, 3 H, 12-CH₃), 4.17 (dd, ³J_{H,F}=49.7 Hz, ²J_{H,H}=3.1 Hz, 1H, 5-CHₜᵣₐₙₛ), 4.42 (d, ²J_{H,H}=16.3 Hz, 1H, 13-CH_{A}), 4.57 (dd, ³J_{H,F}=17.2 Hz, ²J_{H,H}=3.1 Hz, 1H, 5-CH_{cis}), 4.60 (t, ³J_{H,H}=7.3 Hz, 1H, 2-CH), 4.67 (d, ²J_{H,H}=16.3 Hz, 1H, 13-CH_{B}), 6.97 (dm, ³J_{H,H}=9.0 Hz, 2H, 9-CH), 7.29 (dd, ³J_{H,H}=7.9 Hz, ³J_{H,H}=4.9 Hz, 1H, 17-CH), 7.79 (dm, ³J_{H,H}=9.0 Hz, 2H, 10-CH), 7.86 (dd, ³J_{H,H}=7.8 Hz, ⁴J_{H,H}=1.4 Hz, 1H, 18-CH), 8.52-8.54 (m, ⁴J_{H,H}=1.5 Hz, 2H, 15-CH and 16-CH). ¹³C NMR (100.62 MHz, CDCl₃): δ 27.8 (q, C-7), 34.0 (dt, ²J_{H,F}=28.1 Hz, C-3), 47.3 (t, C-13), 55.6 q, C-12), 57.6 (d, C-2), 82.9 (s, C-6), 93.4 (dt, ²J_{H,F}=19.0 Hz, C-5), 114.2 (d, C-9), 123.4 (d, C-17), 129.8 (d, C-10), 131.4 (s, C-8), 132.9 (s, C-14), 136.6 (d, C-18), 148.9 (d, C-16), 149.3 (d, C-15), 161.5 (ds, ¹J_{H,F}=256.5 Hz, C-4), 163.1 (s, C-11), 168.5 (s, C-1). ¹⁹F NMR (470 MHz, CDCl₃): δ −97.6 (dddd, ³J_{H,F}=49.7 Hz, ³J_{H,F}=19.2 Hz, ³J_{H,F}=17.2 Hz, ³J_{H,F}=15.0 Hz, 4-CF).

Optical rotation: [α]₅₈₉²⁰=+7.7, [α]₅₇₈²⁰=+8.1, [α]₅₄₆²⁰=+9.3, [α]₄₃₆²⁰=+16.3, [α]₃₆₅²⁰=+28.4 (c=1.004, CHCl₃).

General Procedure for Hydrolysis of Amino Acid Tert-Butylesters

In a dried YOUNG-tube trifluoroacetic acid (20 mL/mmol) is added under argon to the corresponding amino acid tert-butylester dissolved in dry dichloromethane (20 mL/mmol). After flushing with argon the YOUNG-tube is sealed and the mixture is stirred at r.t. for 3-4 h. Then the reaction mixture was evaporated to dryness. The residue is dissolved in chloroform (100 mL/mmol) and washed with an aqueous solution of citric acid and bicarbonate (pH≈4) (25 mL/mmol). The aqueous phase is extracted with chloroform (4×30 mL/mmol) and the combined organic layer is dried with magnesium sulfate. The solvent is evaporated to get the crude product.

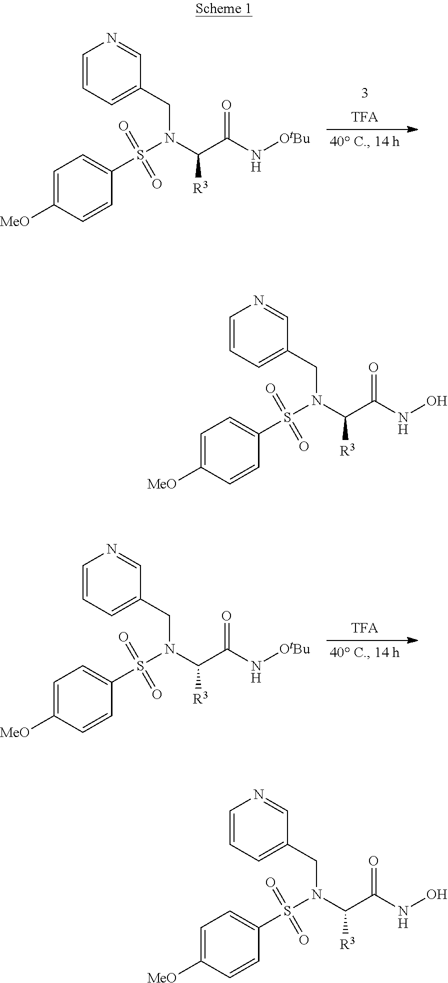

wherein R³ is C₁-C₆-fluoro-alkyl or optionally substituted C₂-C₆ alkenyl wherein the substituent is a F.

### Example 11

### Example 11a

### (S)—N-(4-Methoxyphenylsulfonyl)-N-(3-pyridylmethyl)-aminobutanoic acid

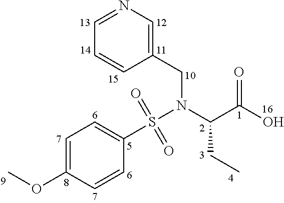

According to the general procedure tert-butyl (S)—N-(4-methoxyphenylsulfonyl)-N-(3-pyridylmethyl)-aminobutanoate (243 mg, 0.58 mmol) was dissolved in dry dichloromethane (12 mL) and treated with trifluoroacetic acid (12 mL). The product was isolated as colorless, highly viscos oil. Yield: 194 mg (91%). ¹H NMR (300 MHz, DMSO-d₆): δ 0.70 (t, ³J_{H,H}=7.3 Hz, 3H, 4-CH₃), 1.45 (ddd, ²J_{H,H}=14.2 Hz, ³J_{H,H}=8.7 Hz, ³J_{H,H}=7.3 Hz, 1H, 3-CH_{A}), 1.75 (dt, ²J_{H,H}=14.0 Hz, ³J_{H,H}=6.8 Hz, 1H, 3-CH_{B}), 3.83 (s, 3H, 9-CH₃), 4.23 (dd, ³J_{H,H}=8.9 Hz, ³J_{H,H}=6.0 Hz, 1H, 2-CH), 4.43 (d, ²J_{H,H}=16.9 Hz, 1H, 10-CH_{A}), 4.61 (d, ²J_{H,H}=16.9 Hz, 1H, 10-CH_{B}), 7.07 (dm, ³J_{H,H}=9.0 Hz, 2H, 6-CH), 7.38 (dd, ³J_{H,H}=7.8 Hz, ³J_{H,H}=4.9 Hz, 1H, 14-CH), 7.74 (dm, ³J_{H,H}=9.0 Hz, 2H, 7-CH), 7.84 (d, ³J_{H,H}=7.9 Hz, 1H, 15-CH), 8.47 (d, ³J_{H,H}=4.1 Hz, 1H, 13-CH), 8.57 (s, 1H, 12-CH). ¹³C NMR (75 MHz, DMSO-d₆): δ 11.0 (q, C-4), 23.2 (t, C-3), 46.4 (t, C-10), 55.7 (q, C-9), 61.5 (d, C-2), 114.3 (d, C-6), 123.4 (d, C-14), 129.5 (d, C-7), 131.0 (s, C-5), 134.3 (s, C-11), 136.3 (d, C-15), 148.0 (d, C-13), 148.9 (d, C-12), 162.6 (s, C-8), 171.7 (s, C-1). Exact mass (ESI⁺): C₁₇H₂₀N₂O₅S+H⁺, calcd. 365.1171. found 365.1169; C₁₇H₂₀N₂O₅S+Na⁺, calcd. 387.0991. found 387.0988; (C₁₇H₂₀N₂O₅S)₂₊H⁺, calcd. 729.2264. found 729.2267; (C₁₇H₂₀N₂O₅S)₂+Na⁺, calcd. 751.2084. found 751.2085. MS (ESI⁺, daughter ion experiment): m/z (%) 365 (90) [M⁺+H⁺], 319 (100) [365−H⁺—CO₂1-1], 280 (65) [C₁₃H₁₆N₂O₃S⁺], 228 (15) [319-C₆H₆N], 200 (30) [228-C₂H₅], 171 (55) [C₇H₇O₃S⁺].

### Example 11b

### (R)—N-(4-Methoxyphenylsulfonyl)-N-(3-pyridylmethyl)-aminobutanoic acid

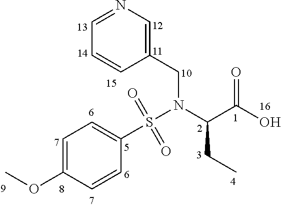

According to the general procedure tert-butyl (R)—N-(4-methoxyphenylsulfonyl)-N-(3-pyridylmethyl)-aminobutanoate (133 mg, 0.32 mmol) dissolved in dry dichloromethane (8 mL) and treated with trifluoroacetic (8 mL) acid. The product was isolated as colorless, highly viscos oil. Yield: 112 mg (96%). ¹H NMR (300 MHz, DMSO-d₆): δ 0.71 (t, ³J_{H,H}=7.3 Hz, 3 H, 4-CH₃), 1.59-1.87 (m, 2H, 3-CH₂), 3.84 (s, 3H, 9-CH₃), 4.25 (dd, ³J_{H,H}=8.8 Hz, ³J_{H,H}=6.0 Hz, 1H, 2-CH), 4.52 (d, ²J_{H,H}=17.1 Hz, 1H, 10-CH_{A}), 4.66 (d, ²J_{H,H}=17.1 Hz, 1H, 10-CH_{B}), 7.08 (dm, ³J_{H,H}=8.9 Hz, 2H, 6-CH), 7.65 (m, 1H, 14-CH), 7.76 (dm, ³J_{H,H}=8.9 Hz, 2H, 7-CH), 8.10 (d, ³J_{H,H}=7.8 Hz, 1H, 15-CH), 8.45-8.95 (m, 2H, 12-CH & 13-CH), 12.44 (s br, 1H, 16-OH). ¹³C-NMR (75 MHz, DMSO-d₆): δ 11.0 (q, C-4), 23.2 (t, C-3), 46.2 (t, C-10), 55.7 (q, C-9), 61.5 (d, C-2), 114.3 (d, C-6), 124.6 (d, C-14), 129.5 (d, C-7), 130.4 (s, C-11), 130.8 (s, C-5), 139.0 (d, C-15), 145.5 (d, C-13), 146.2 (d, C-12), 162.7 (s, C-8), 171.7 (s, C-1). Exact mass (ESI⁺): C₁₇H₂₀N₂O₅S+H⁺, calcd. 365.1171, 365.1166; C₁₇H₂₀N₂O₅S+Na⁺, calcd. 387.0991. found 387.0986; (C₁₇H₂₀N₂O₅S)₂+H⁺, calcd. 729.2264. found 729.2267; (C₁₇H₂₀N₂O₅S)₂+Na⁺, calcd. 751.2084. found 751.2085. MS (ESI⁺, daughter ion experiment): m/z (%) 365 (100) [M⁺+H⁺], 319 (32) [365-H⁺—CO₂H], 280 (20) [C₁₃H₁₆N₂O₃S⁺], 228 (5) [319-C₆H₆N], 200 (10) [228-C₂H₅], 171 (18) [C₇H₇O₃S⁺], 149 (38) [319-C₇H₇O₃S].

### Example 12

### Example 12a

### (S)—N-(4-Methoxyphenylsulfonyl)-N-(3-pyridylmethyl)-aminopent-4-enoic acid

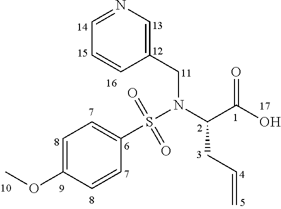

According to the general procedure tert-butyl (S)—N-(4-methoxyphenylsulfonyl)-N-(3-pyridylmethyl)-2-aminopent-4-enoate (400 mg, 0.93 mmol) was dissolved in dry dichloromethane (20 mL) and treated with trifluoroacetic acid (20 mL). The product was isolated as colorless, highly viscos oil. Yield: 312 mg (89%). ¹H NMR (400 MHz, DMSO-d₆): δ 2.16 (ddd, ²J_{H,H}=15.0 Hz, ³J_{H,H}=8.4 Hz, ³J_{H,H}=7.4 Hz, 1H, 3-CH_{A}), 2.42 (m, 1H, 3-CH_{B}), 3.76 (s, 3H, 10-CH₃), 4.35 (dd, ³J_{H,H}=8.7 Hz, ²J_{H,H}=6.2 Hz, 1H, 2-CH), 4.36 (d, ²J_{H,H}=17.3 Hz, 1H, 11-CH_{A}), 4.51 (d, ²J_{H,H}=16.9 Hz, 1H, 11-CH_{B}), 4.80 (dd, ³J_{H,H}=17.2 Hz, ²J_{H,H}=1.6 Hz, 1H, 5-CHₜᵣₐₙₛ), 4.84 (dd, ³J_{H,H}=10.3 Hz, ²J_{H,H}=1.5 Hz, 1H, 5-CH_{cis}), trans, 5.49 (ddt, ³J_{H,H}=17.0 Hz, ³J_{H,H}=10.3 Hz, ³J_{H,H}=6.7 Hz, 1H, 4-CH), 6.99 (dm, ³J_{H,H}=9.0 Hz, 2H, 7-CH), 7.25 (dd, ³J_{H,H}=7.8 Hz, ³J_{H,H}=4.8 Hz, 1H, 15-CH), 7.66 (dm, ³J_{H,H}=8.9 Hz, 2H, 8-CH), 7.70 (m, 1H, 16-CH), 8.36 (d, ³J_{H,H}=4.1 Hz, 1H, 14-CH), 8.46 (s, 1H, 13-CH). ¹³C NMR (100 MHz, DMSO-d₆): δ 34.1 (t, C-3), 46.5 (t, C-11), 55.7 (q, C-10), 59.7 (d, C-2), 114.3 (d, C-7), 117.8 (t, C-5), 123.1 (d, C-15), 129.5 (d, C-8), 131.0 (s, C-6), 133.8 (d, C-4), 134.0 (s, C-12), 135.9 (d, C-16), 148.1 (d, C-14), 149.2 (d, C-13), 162.6 (s, C-9), 171.2 (s, C-1). Exact mass (ESI⁺): C₁₈H₂₀N₂O₅S+H⁺, calcd. 377.1171. found 377.1186; C₁₈H₂₀N₂O₅S+Na⁺, calcd. 399.0991. found 399.1004; (C₁₈H₂₀N₂O₅S)₂+H⁺, calcd. 753.2264. found 753.2259; (C₁₈H₂₀N₂O₅S)₂+Na⁺, calcd. 775.2084. found 775.2094. Exact mass (ESI⁻): C₁₈H₂₀N₂O₅S—H⁺, calcd. 375.1015. found 375.1035; (C₁₈H₂₀N₂O₅S)-2—H⁺, calcd. 751.2108. found 751.2141. MS (ESI⁺, daughter ion experiment): m/z (%) 377 (75) [M⁺+H⁺], 331 (75) [377-H⁺—CO₂H], 280 (42) [C₁₃H₁₆N₂O₃S⁺], 240 (8) [331-C₆H₆N], 200 (20) [C₈H₁₀NO₃S⁺], 171 (50) [C₇H₇O₃S⁺], 161 (100) [331-C₇H₇O₃S], 123 (8) [C₇H₇O₂⁺].

### Example 12b

### (R)—N-(4-Methoxyphenylsulfonyl)-N-(3-pyridylmethyl)-aminopent-4-enoic acid

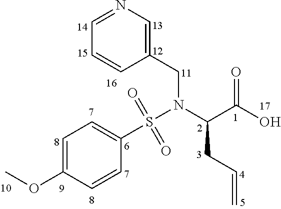

According to the general procedure tert-butyl (R)—N-(4-methoxyphenylsulfonyl)-N-(3-pyridylmethyl)-2-aminopent-4-enoate (247 mg, 0.57 mmol) was dissolved in dry dichloromethane (12 mL) and treated with trifluoroacetic acid (12 mL). The product was isolated as white highly viscos oil. Yield: 196 mg (92%). ¹H NMR (400 MHz, DMSO-d₆): δ 2.29 (ddd, ²J_{H,H}=15.0 Hz, ³J_{H,H}=8.6 Hz, ³J_{H,H}=7.6 Hz, 1H, 3-CH_{A}), 2.54 (m, ²J_{H,H}=15.0 Hz, ³J_{H,H}=6.1 Hz, 1H, 3-CH_{B}), 3.84 (s, 3H, 10-CH₃), 4.48 (dd, ³J_{H,H}=8.9 Hz, ³J_{H,H}=6.0 Hz, 1H, 2-CH), 4.53 (d, ²J_{H,H}=17.3 Hz, 1H, 11-CH_{A}), 4.65 (d, ²J_{H,H}=17.2 Hz, 1H, 11-CH_{B}), 4.89 (dd, ³J_{H,H}=17.2 Hz, ²J_{H,H}=1.7 Hz, 1H, 5-CHₜᵣₐₙₛ), 4.93 (dd, ³J_{H,H}=10.5 Hz, ²J_{H,H}=1.6 Hz, 1H, 5-CH_{cis}), 5.58 (ddt, ³J_{H,H}=17.1 Hz, ³J_{H,H}=10.3 Hz, ³J_{H,H}=6.7 Hz, 1H, 4-CH), 7.09 (d, ³J_{H,H}=9.0 Hz, 2H, 7-CH), 7.58 (dd, ³J_{H,H}=7.9 Hz, ³J_{H,H}=5.1 Hz, 1H, 15-CH), 7.75 (d, ³J_{H,H}=9.0 Hz, 2H, 8-CH), 8.07 (d, ³J_{H,H}=8.0 Hz, 1H, 16-CH), 8.59 (d, ³J_{H,H}=4.4 Hz, 1H, 14-CH), 8.67 (s, 1H, 13-CH), 12.82 (s br, 1H, 17-OH). ¹³C NMR (100 MHz, DMSO-d₆): δ 34.0 (t, C-3), 46.3 (t, C-11), 55.7 (q, C-10), 59.7 (d, C-2), 114.3 (d, C-7), 117.8 (t, C-5), 124.3 (d, C-15), 129.6 (d, C-8), 130.7 (s, C-6), 134.0 (d, C-4), 135.7 (s, C-12), 139.1 (d, C-16), 145.6 (d, C-14), 146.4 (d, C-13), 162.7 (s, C-9), 171.2 (s, C-1).

### Example 13

### Example 13a

### (S)—N-(4-Methoxyphenylsulfonyl)-N-(3-pyridylmethyl)-2-amino-4-fluorobutanoic acid

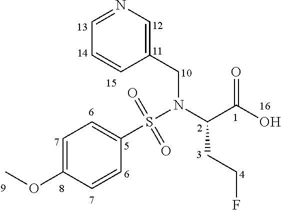

According to the general procedure tert-butyl (S)—N-(4-methoxyphenylsulfonyl)-N-(3-pyridylmethyl)-2-amino-4-fluorobutanoate (562 mg, 1.28 mmol) was dissolved in dry dichloromethane (5 mL) and treated with trifluoroacetic acid (25 mL). The product was isolated as white solid. Yield: 472 mg (96%). M.p. 156° C. ¹H NMR (300 MHz, DMSO-d₆): δ 1.85 (m, ³J_{H,F}=22.3 Hz, 1H, 3-CH_{A}), 2.19 (m, ³J_{H,F}=27.9 Hz, 1H, 3-CH_{B}), 3.84 (s, 3H, 9-CH₃), 4.32 (dt, ²J_{H,F}=47.1 Hz, ³J_{H,H}=5.7 Hz, 2H, 4-CH₂), 4.43 (d, ²J_{H,H}=17.2 Hz, 1H, 10-CH_{A}), 4.49 (m, 1H, 2-CH), 4.62 (d, ²J_{H,H}=16.8 Hz, 1H, 10-CH_{B}), 7.09 (dm, ³J_{H,H}=8.9 Hz, 2H, 6-CH), 7.39 (dd, ³J_{H,H}=7.8 Hz, ³J_{H,H}=4.8 Hz, 1H, 14-CH), 7.75 (dm, ³J_{H,H}=8.9 Hz, 2H, 7-CH), 7.82 (m, 1H, 15-CH), 8.49 (d, ³J_{H,H}=3.7 Hz, 1H, 13-CH), 8.55 (s, 1H, 12-CH), 13.02 (s br, 1H, 16-OH). ¹³C NMR (75 MHz, DMSO-d₆): δ 30.9 (dt, ²J_{H,F}=20.2 Hz, C-3), 46.8 (t, C-10), 55.7 (q, C-9), 56.3 (dd, ³J_{H,F}=5.0 Hz, C-2), 80.4 (dt, ¹J_{H,F}=163.1 Hz, C-4), 114.4 (d, C-6), 123.5 (d, C-14), 129.6 (d, C-7), 130.8 (s, C-5), 133.9 (s, C-11), 136.4 (d, C-15), 148.0 (d, C-13), 148.8 (d, C-12), 162.7 (s, C-8), 171.2 (s, C-1). ¹⁹F NMR (282 MHz, DMSO-d₆): δ −219.3 (ddt, ²J_{H,F}=47.1 Hz, ³J_{H,F}=27.8 Hz, ³J_{H,F}=22.3 Hz, 4-CH₂F). Exact mass (ESI⁺): C₁₇H₁₉FN₂O₅S+H⁺, calcd. 383.1077. found 383.1073; C₁₇H₁₉FN₂O₅S+Na⁺. found 405.0896. found 405.0892; (C₁₇H₁₉FN₂O₅S)₂+H⁺, calcd. 765.2076. found 765.2071; (C₁₇H₁₉FN₂O₅S)₂₊Na⁺, calcd. 787.1895. found 787.1887. Exact mass (ESI⁻): C₁₇H₁₉FN₂O₅S—H⁺, calcd. 381.0926. found 381.0930; (C₁₇H₁₉FN₂O₅S—H)₂+H⁺, calcd. 763.1925. found 763.1930; (C₁₇H₁₉FN₂O₅S—H⁺)₂+Na⁺, calcd. 785.1744. found 785.1758.

### Example 13b

### (R)—N-(4-Methoxyphenylsulfonyl)-N-(3-pyridylmethyl)-2-amino-4-fluorobutanoic acid

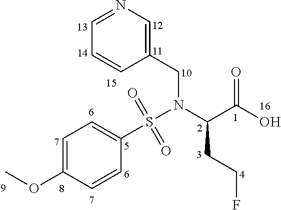

According to the general procedure tert-butyl (R)—N-(4-methoxyphenylsulfonyl)-N-(3-pyridylmethyl)-2-amino-4-fluorobutanoate (448 mg, 1.02 mmol) was dissolved in dry dichloromethane (25 mL) and treated with trifluoroacetic acid (25 mL). The product was isolated as white solid. Yield: 352 mg (90%). M.p. 155-156° C. ¹H NMR (400 MHz, DMSO-d₆): δ 1.79 (m, ³J_{H,F}=22.8 Hz, 1H, 3-CH_{A}), 2.19 (m, ³J_{H,F}=27.6 Hz, 1H, 3-CH_{B}), 3.84 (s, 3H, 9-CH₃), 4.32 (t, ²J_{H,H}=5.7 Hz, 1H, 4-CH_{A}), 4.37 (t, ²J_{H,H}=5.7 Hz, 1H, 4-CH_{B}), 4.41 (d, ²J_{H,H}=17.1 Hz, 1H, 10-CH_{A}), 4.48 (t, ³J_{H,H}=7.2 Hz, 1H, 2-CH), 4.61 (d, ²J_{H,H}=16.7 Hz, 1H, 10-CH_{B}), 7.09 (dm, ³J_{H,H}=8.5 Hz, 2H, 6-CH), 7.34 (dd, ³J_{H,H}=7.6 Hz, ³J_{H,H}=4.9 Hz, 1H, 14-CH), 7.72-7.80 (m, 3H, 7-CH and 15-CH), 8.46 (d, ³J_{H,H}=4.7 Hz, 1 H, 13-CH), 8.52 (s, 1H, 12-CH), 13.11 (s br, 1H, 16-OH). ¹³C NMR (100 MHz, DMSO-d₆): δ 31.0 (dt, ²J_{H,F}=20.2 Hz, C-3), 46.8 (t, C-10), 55.7 (q, C-9), 56.2 (dd, ³J_{H,F}=5.0 Hz, C-2), 80.4 (dt, ¹J_{H,F}=163.1 Hz, C-4), 114.3 (d, C-6), 123.2 (d, C-14), 129.5 (d, C-7), 130.9 (s, C-5), 133.6 (s, C-11), 135.8 (d, C-15), 148.5 (d, C-13), 149.3 (d, C-12), 162.7 (s, C-8), 171.2 (s, C-1). ¹⁹F NMR (282 MHz, DMSO-d₆): δ −221.8 (ddt, ²J_{H,F}=47.1 Hz, ²J_{H,F}=27.5 Hz, ²J_{H,F}=22.5 Hz, 4-CH₂F). Exact mass (ESI⁺): C₁₇H₁₉FN₂O₅S+H⁺, calcd. 383.1077. found 383.1079; C₁₇H₁₉FN₂O₅S+Na⁺, calcd. 405.0896. found 405.0900; (C₁₇H₁₉FN₂O₅S)₂+H⁺, calcd. 765.2076. found 765.2071; (C₁₇H₁₉FN₂O₅S)₂+Na⁺, calcd. 787.1895. found 787.1884. MS (ESI⁺, daughter ion experiment): m/z (%) 383 (75) [M⁺+H⁺], 363 (4) [M⁺-HF], 337 (22) [383-H⁺—CO₂H], 280 (40) [C₁₃H₁₆N₂O₃S⁺], 211 (5) [383-C₇H₇O₃S], 200 (13) [C₈H₁₀NO₃S⁺], 171 (18) [C₇H₇O₃S⁺], 167 (100) [211-CO₂H], 123 (8) [C₇H₇O₂⁺].

### Example 14

### Example 14a

### (S)—N-(4-Methoxyphenylsulfonyl)-N-(3-pyridylmethyl)-2-amino-4-fluoropent-4-enoic acid

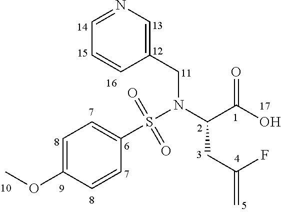

According to the general procedure tert-butyl (S)—N-(4-Methoxyphenylsulfonyl)-N-(3-pyridylmethyl)-2-amino-4-fluoropent-4-enoate (272 mg, 0.60 mmol) was dissolved in dry dichloromethane (20 mL) and treated with 20 mL trifluoroacetic acid. The product was isolated as yellowish-white solid. Yield: 200 mg (85%). M.p. 172° C. ¹H NMR (300 MHz, DMSO-d₆): δ 2.59 (m, 1H, 3-CH_{A}), 2.84 (ddd, ³J_{H,F}=15.7 Hz, ²J_{H,H}=12.6 Hz, ³J_{H,H}=5.5 Hz, 1H, 3-CH_{B}), 3.91 (s, 3H, 10-CH₃), 4.30 (dd, ³J_{H,F}=51.6 Hz, ²J_{H,H}=3.1 Hz, 1H, 4.53 (d, ²J_{H,H}=16.9 Hz, 1H, 11-CH_{A}), 4.62 (dd, ³J_{H,F}=17.8 Hz, ²J_{H,H}=3.0 Hz, 1H, 5-CH_{cis}), 4.66 (d, ²J_{H,H}=16.8 Hz, 1H, 11-CH_{B}), 4.69 (dd, ³J_{H,H}=5.7 Hz, ³J_{H,H}=3.0 Hz, 1H, 2-CH), 7.14 (d, ³J_{H,H}=9.1 Hz, 2H, 7-CH), 7.45 (m, 1H, 15-CH), 7.80 (d, ³J_{H,H}=9.0 Hz, 2 H, 8-CH), 7.88 (m, 1H, 16-CH), 8.55 (s, 1H, 14-CH), 8.63 (s, 1H, 13-CH), 12.94 (s, 1H, 17-OH). ¹³C NMR (75 MHz, DMSO-d₆): δ 32.6 (dt, ²J_{H,F}=28.0 Hz, C-3), 46.7 (t, C-11), 55.7 (q, C-10), 57.0 (d, C-2), 93.2 (dt, ²J_{H,F}=18.4 Hz, C-5), 114.3 (d, C-7), 123.4 (d, C-15), 129.4 (d, C-8), 129.6 (s, C-6), 130.7 (s, C-12), 136.6 (d, C-16), 147.7 (d, C-14), 148.7 (d, C-13), 161.7 (ds, ¹J_{H,F}=255.3 Hz, C-4), 162.7 (s, C-9), 169.9 (s, C-1). ¹⁹F NMR (282 MHz, DMSO-d₆): δ −96.2 (m, ³J_{H,F}=51.5 Hz, ³J_{H,F}=17.8 Hz, ³J_{H,F}=15.7 Hz, 4-CF). Exact mass (ESI⁺): C₁₈H₁₉FN₂O₅S+H⁺, calcd. 395.1077. found 395.1088; C₁₈H₁₉FN₂O₅S+Na⁺, calcd. 417.0896. found 417.0905; (C₁₈H₁₉FN₂O₅S)₂+H⁺, calcd. 789.2076. found 789.2096; (C₁₈H₁₉FN₂O₅S)₂+Na⁺, calcd. 811.1895. found 811.1897. Exact mass (ESI⁻): C₁₈H₁₉FN₂O₅S—H⁺, calcd. 393.0920. found 393.0940; (C₁₈H₁₉FN₂O₅S—H⁺)₂+H⁺, calcd. 787.1919. found 787.1955; (C₁₈H₁₉FN₂O₅S—H⁺)₂+Na⁺, calcd. 809.1739. found 809.1766. MS (ESI⁺, daughter ion experiment): m/z (%) 395 (12) [M⁺+H⁺], 179 (22) [M⁺-C₂H₂F—C₇H₇O₃S], 171 (31) [C₇H₇O₃S⁺], 165 (100) [M⁺-C₃H₄F—C₇H₇O₃S], 147 (10), 123 (8), 107 (18) [C₇H₇O⁺], 92 (20) [C₆H₇N⁺]. MS (ESI⁻, daughter ion experiment): m/z (%) 393 (2) [M⁻-H⁺], 277 (27) [393-C₅H₅FO₂⁻], 171 (100) [C₇H₇O₃S⁻], 157 (4) [C₆H_{S}O₃S⁻], 115 (18) [C₅H₄FO₂⁻].

### Example 14b

### (R)—N-(4-Methoxyphenylsulfonyl)-N-(3-pyridylmethyl)-2-amino-4-fluoropent-4-enoic acid

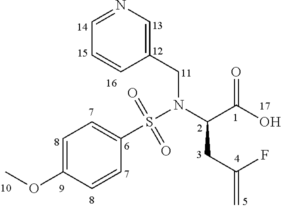

According to the general procedure tert-butyl (R)—N-(4-methoxyphenylsulfonyl)-N-(3-pyridylmethyl)-2-amino-4-fluoropent-4-enoate (256 mg, 0.57 mmol) was dissolved in dry dichloromethane (15 mL) and treated with trifluoroacetic acid (15 mL). The product was isolated as yellowish-white solid. Yield: 204 mg (91%). M.p. 172-173° C. ¹H NMR (300 MHz, DMSO-d₆): δ 2.51 (m, 1H, 3-CH_{A}), 2.77 (ddd, ³J_{H,F}=21.2 Hz, ²J_{H,H}=12.7 Hz, ³J_{H,H}=5.8 Hz, 1H, 3-CH_{B}), 4.22 (dd, ³J_{H,F}=51.5 Hz, ²J_{H,H}=3.0 Hz, 1H, 5-CHₜᵣₐₙₛ), 4.45 (d, ²J_{H,H}=16.8 Hz, 1H, 11-CH_{A}), 4.55 (dd, ³J_{H,F}=17.8 Hz, ²J_{H,H}=3.0 Hz, 1H, 5-CH_{cis}), 4.59 (d, ²J_{H,H}=16.5 Hz, 1H, 11-CH_{B}), 4.62 (t, ³J_{H,H}=4.3 Hz, 1H, 2-CH), 7.07 (d, ³J_{H,H}=9.0 Hz, 2H, 7-CH), 7.33 (dd, ³J_{H,H}=7.7 Hz, ³J_{H,H}=4.8 Hz, 1H, 15-CH), 7.73 (d, ³J_{H,H}=9.0 Hz, 2H, 8-CH), 7.79 (m, 1H, 16-CH), 8.45 (d, ³J_{H,H}=4.6 Hz, 1H, 14-CH), 8.54 (s, 1H, 13-CH), 12.89 (s br, 1H, 17-OH). ¹³C NMR (75.48 MHz, DMSO-d₆): δ 32.7 (dt, ²J_{H,F}=27.6 Hz, C-3), 46.7 (t, C-11), 55.7 (q, C-10), 56.9 (d, C-2), 93.2 (dt, ²J_{H,F}=18.1 Hz, C-5), 114.3 (d, C-7), 123.2 (d, C-15), 129.6 (d, C-8), 130.8 (s, C-6), 132.8 (s, C-12), 136.1 (d, C-16), 148.1 (d, C-14), 149.1 (d, C-13), 161.7 (ds, ¹J_{H,F}=255.3 Hz, C-4), 162.7 (s, C-9), 170.5 (s, C-1). ¹⁹F NMR (282.37 MHz, DMSO-d₆): δ −96.2 (dddd, ³J_{H,F}=51.5 Hz, ³J_{H,F}=21.1 Hz, ³J_{H,F}=17.9 Hz, ³J_{H,F}=12.6 Hz, 4-CF). Exact mass (ESI⁺): C₁₈H₁₉FN₂O₅S+H⁺, calcd. 395.1077. found 395.1080; C₁₈H₁₉FN₂O₅S+Na⁺, calcd. 417.0896. found 417.0896. Exact mass (ESI⁻): C₁₈H₁₉FN₂O₅S—H⁺, calcd. 393.0920. found 393.0948; (C₁₈H₁₉FN₂O₅S)₂—H⁺, calcd. 787.1919. found 787.1940. MS (ESI⁺, daughter ion experiment): m/z (%) 395 (45) [M⁺+H⁺], 375 (5) [395-HF], 349 (22) [395-H⁺—CO₂H], 305 (20) [349-C₂H₂F], 280 (30) [C₁₃H₁₆N₂O₃S⁺], 200 (10) [305-C₇H₇O], 179 (100) [C₆H₁₀FNO₂S⁺], 171 (75) [C₇H₇O₃S⁺].

General Procedure for Hydrolysis of Amino Acid Methylesters

The aminoacid methylester (1 equivalent) dissolved in a mixture of THF, methanol and water (ratio 3:1:1; 3.3 mL/mmol) is treated with lithium hydroxide (5 equivalents). This solution is stirred at r.t. over night. The reaction mixture is extracted with ethyl acetate (2×20 mL). This extract is discharged. Then the aqueous phase is acidified with 0.5 N hydrochloric acid and extracted again with ethyl acetate (4×20 mL). The combined organic phases are dried with magnesium sulfate and the solvent is evaporated.

### Example 15

### Example 15a (S)—N-(4-Methoxyphenylsulfonyl)-N-(3-pyridylmethyl)aminobutanoic acid

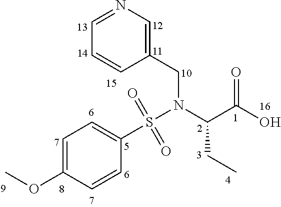

According to the general procedure methyl (S)—N-(4-methoxyphenylsulfonyl)-N-(3-pyridylmethyl)-aminobutanoate (306 mg, 0.81 mmol) was dissolved in the above-mentioned solvent mixture (10 mL) and treated with lithium hydroxide (169 mg, 4.04 mmol). The product was isolated as white, highly viscos liquid. Yield: 174 mg (59%). The spectroscopic data agree with those given above.

### Example 15b

### (R)—N-(4-Methoxyphenylsulfonyl)-N-(3-pyridylmethyl)aminobutanoic acid

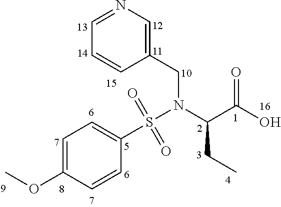

According to the general procedure methyl (R)—N-(4-methoxyphenylsulfonyl)-N-(3-pyridylmethyl)-aminobutanoate (420 mg, 1.11 mmol) was dissolved in the above-mentioned solvent mixture (10 mL) and treated with lithium hydroxide (230 mg, 5.55 mmol). The product was isolated as white, highly viscos liquid. Yield: 251 mg (62%). The spectroscopic data agree with those given above.

Synthesis of the Hydroxamic Acids

General Procedure for Synthesis of the Hydroxamates

The N,N-disubstituted α-amino acid (1 equivalent) is suspended in dichloromethane (15 mL/mmol). Then 1-hydroxy-benzotriazole (HOBT, 1 equivalent) is added and stirred for a short time. After addition of N-methylmorpholin (NMM) (5 equiv.) the acid is dissolved. Subsequently, O-tert-butylhydroxylamine (3 equiv.) was added and the solution is stirred for 5 min 1-(3-Dimethylaminopropyl)-3-ethylcarbodiimide (EDC) hydrochloride (1.3 equiv.) is added and the reaction mixture is stirred overnight. The solution is washed with water (20 mL) and the aqueous phase is extracted with dichloromethane (3×20 mL). The combined organic phases are washed with brine (1×20 mL) and dried with magnesium sulfate. After evaporation of the solvent the crude product is purified by column chromatography (silica gel, Cy/EtOAc, 1:3). The silica gel was inactivated by flushing with the eluent containing 2% triethylamine. The product is obtained as highly viscose liquid, which on drying in high vacuum becomes solid.

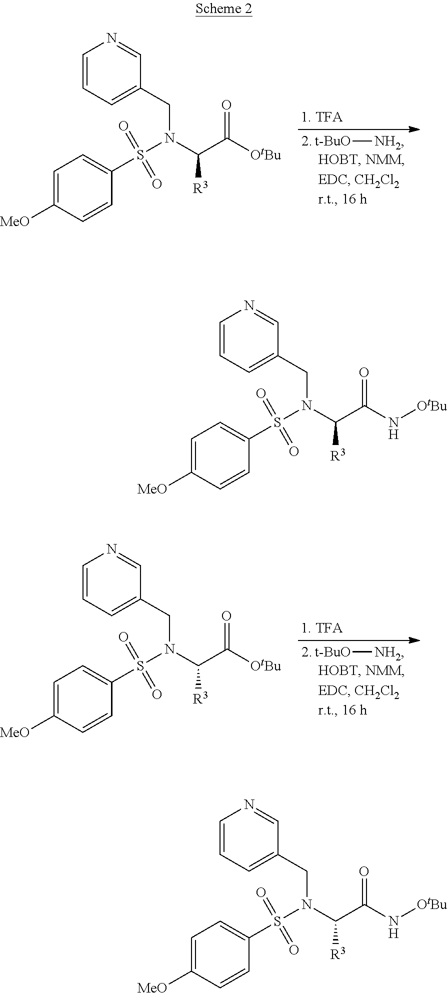

### Example 16

### Example 16a Tert-butyl (S)—N-(4-methoxyphenylsulfonyl)-N-(3-pyridylmethyl)-2-aminobutan-hydroxamate

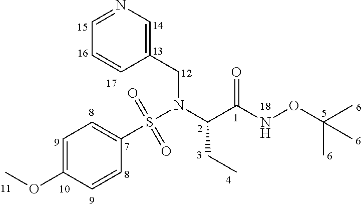

According to the general procedure (S)—N-(4-methoxyphenylsulfonyl)-N-(3-pyridylmethyl)aminobutanoic acid (185 mg, 0.51 mmol) was reacted with HOBT (71 mg, 0.51 mmol), NMM (259 mg, 2.55 mmol), O-tert-butylhydroxylamine (194 mg, 1.53 mmol) and EDC (134 mg, 0.69 mmol) in dichloromethane (15 mL). After purification by column chromatography (column Ø 2 cm×15) the product was isolated as white solid. Yield: 173 mg (78%). M.p. 66° C. ¹H NMR (300 MHz, DMSO-d₆): δ 0.69 (t, ³J_{H,H}=7.2 Hz, 3H, 4-CH₃), 1.23 (s, 9H, 6-CH₃), 1.44 (tq, ²J_{H,H}=14.8 Hz, ³J_{H,H}=7.4 Hz, 1H, 3-CH_{A}), 1.90 (m, 1H, 3-CH_{B}), 3.85 (s, 3H, 11-CH₃), 4.11 (m, 1H, 2-CH), 4.56 (s, 2H, 12-CH₂), 6.91 (d, ³J_{H,H}=8.9 Hz, 2H, 8-CH), 7.20 (dd, ³J_{H,H}=7.8 Hz, ³J_{H,H}=4.9 Hz, 1H, 16-CH), 7.61 (d, ³J_{H,H}=8.9 Hz, 2H, 9-CH), 7.71 (d, ³J_{H,H}=7.8 Hz, 1H, 17-CH), 8.48 (d, ³J_{H,H}=3.7 Hz, 1H, 15-CH), 8.54 (d, ⁴J_{H,H}=1.5 Hz, 1H, 14-CH), 8.59 (s, 1H, 18-NH). ¹³C NMR (75 MHz, DMSO-d₆): δ 10.6 (q, C-4), 22.9 (t, C-3), 26.2 (q, C-6), 45.7 (t, C-12), 55.6 (q, C-11), 59.5 (d, C-2), 82.2 (s, C-5), 114.4 (d, C-8), 123.2 (d, C-16), 129.2 (d, C-9), 131.3 (s, C-7), 132.9 (s, C-13), 136.5 (d, C-17), 148.8 (d, C-15), 149.9 (d, C-14), 163.2 (s, C-10), 168.2 (s, C-1). Exact mass (ESI⁺): C₂₁H₂₉N₃O₅S+H⁺, calcd. 436.1906. found 436.1898; C₂₁H₂₉N₃O₅S+Na⁺, calcd. 458.1726. found 458.1716; (C₂₁H₂₉N₃O₅S)₂+H⁺, calcd. 871.3734. found 871.3717; (C₂₁H₂₉N₃O₅S)₂+Na⁺, calcd. 893.3554. found 893.3534. MS (ESI⁺, daughter ion experiment): m/z (%)=436 (50) [M⁺+H⁺], 380 (100) [436-C₄H₈], 319 (18) [436-H⁺—CONHOC(CH₃)₃], 264 (6) [436-H⁺—C₇H₇O₃S], 228 (4) [319-C₆H₆N], 171 (12) [C₇H₇O₃S⁺]. Optical rotation: [α]₅₈₉²⁰=−37.2, [α]₅₇₈²⁰=−39.3, [α]₅₄₆²⁰=−45.2, [α]₄₃₆²⁰=−84.7, [α]₃₆₅²⁰=−156.2 (c=1.007, CHCl₃).

### Example 16b

### Tert-butyl (R)—N-(4-methoxyphenylsulfonyl)-N-(3-pyridylmethyl)-2-aminobutanhydroxamate

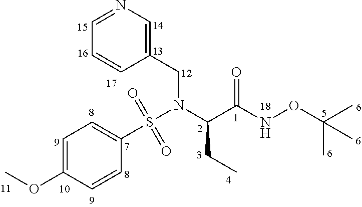

According to the general procedure (R)—N-(4-methoxyphenylsulfonyl)-N-(3-pyridylmethyl)-aminobutanoic acid (105 mg, 0.29 mmol) was reacted with HOBT (40 mg, 0.29 mmol), NMM (147 mg, 1.45 mmol), O-tert-butylhydroxylamine (110 mg, 0.87 mmol) and EDC (74 mg, 0.38 mmol) in dichloromethane (15 mL). After column chromatography (column Ø 2 cm×15) the product was isolated as white solid. Yield: 101 mg (83%). M.p. 65-66° C. ¹H NMR (300.13 MHz, DMSO-d₆): δ 0.69 (t, ³J_{H,H}=7.3 Hz, 3H, 4-CH₃), 1.25 (s, 9H, 6-CH₃), 1.44 (m, 1H, 3-CH_{A}), 1.90 (m, 1H, 3-CH_{B}), 3.85 (s, 3H, 11-CH₃), 4.09 (m, 1H, 2-CH), 4.56 (s, 2H, 12-CH₂), 6.91 (dm, ³J_{H,H}=8.9 Hz, 2H, 8-CH), 7.20 (dd, ³J_{H,H}=7.8 Hz, ³J_{H,H}=4.8 Hz, 1H, 16-CH), 7.61 (dm, ³J_{H,H}=8.9 Hz, 2H, 9-CH), 7.70 (d, ³J_{H,H}=7.8 Hz, 1H, 17-CH), 8.49 (d, ³J_{H,H}=3.7 Hz, 1H, 15-CH), 8.53-8.56 (m, 2H, 14-CH and 18-NH). ¹³C NMR (75.48 MHz, DMSO-d₆): δ 10.6 (q, C-4), 22.9 (t, C-3), 26.2 (q, C-6), 45.7 (t, C-12), 55.6 (q, C-11), 59.5 (d, C-2), 82.2 (s, C-5), 114.4 (d, C-8), 123.2 (d, C-16), 129.2 (d, C-9), 131.4 (s, C-7), 132.9 (s, C-13), 136.5 (d, C-17), 148.9 (d, C-15), 150.0 (d, C-14), 163.2 (s, C-10), 168.2 (s, C-1). Exact mass (ESI⁺): C₂₁H₂₉N₃O₅S)₂+Na⁺, calcd. 436.1906. found 436.1897; C₂₁H₂₉N₃O₅S+Na⁺, calcd. 458.1726. found 458.1715; (C₂₁H₂₉N₃O₅S)₂+H⁺, calcd. 871.3734. found 871.3702; (C₂₁H₂₉N₃O₅S)₂+Na⁺, calcd. 893.3554. found 893.3521. MS (ESI⁺, daughter ion experiment): m/z (%) 436 (65) [M⁺+H⁺], 380 (100) [436-C₄H₈], 319 (12) [436-H⁺-CONHOC(CH₃)₃], 264 (15) [436-H⁺—C₇H₇O₃S], 228 (5) [319-C₆H₆N]. Optical rotation: [α]₅₈₉²⁰=+37.2, [α]₅₇₈²⁰=+39.1, [α]₅₄₆²⁰=+44.9, [α]₄₃₆²⁰=+84.3, [α]₃₆₅²⁰=+155.4 (c=1.004, CHCl₃).

### Example 17

### Example 17a

### Tert-butyl (S)—N-(4-methoxyphenylsulfonyl)-N-(3-pyridylmethyl)-2-aminopent-4-enhydroxamate

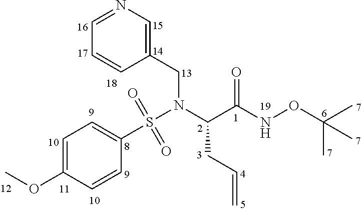

According to the general procedure (S)—N-(4-methoxyphenylsulfonyl)-N-(3-pyridylmethyl)-aminopent-4-enoic acid (280 mg, 0.744 mmol) was reacted with HOBT (101 mg, 0.744 mmol), NMM (377 mg, 3.72 mmol), O-tert-butylhydroxylamine (280 mg, 2.23 mmol) and EDC (186 mg, 0.97 mmol) in dichloromethane (15 mL). After column chromatography (column Ø 2 cm×15 cm) the product was isolated as white solid. Yield: 306 mg (92%). M.p. 65-66° C. ¹H NMR (300 MHz, CDCl₃): δ 1.21 (s, 9H, 7-CH₃), 2.16 (dt, ²J_{H,H}=13.8 Hz, ³J_{H,H}=6.8 Hz, 1H, 3-CH_{A}), 2.60 (ddd, ²J_{H,H}=14.0 Hz, ³J_{H,H}=8.1 Hz, ³J_{H,H}=7.0 Hz, 1 H, 3-CH_{B}), 3.86 (s, 3H, 12-CH₃), 4.29 (dd, ³J_{H,H}=8.3 Hz, ³J_{H,H}=6.9 Hz, 1H, 2-CH), 4.58 (s, 2H, 13-CH₂), 4.94 (d, ²J_{H,H}=2.9 Hz, 1H, 5-CH_{A}), 4.97 (d, ²J_{H,H}=3.1 Hz, 1H, 5-CH_{B}), 5.39 (ddt, ³J_{H,H}=14.1 Hz, ³J_{H,H}=9.6 Hz, ³J_{H,H}=6.9 Hz, 1H, 4-CH), 6.93 (dm, ³J_{H,H}=8.9 Hz, 2H, 9-CH), 7.21 (dd, ³J_{H,H}=7.7 Hz, ³J_{H,H}=4.9 Hz, 1H, 17-CH), 7.64 (dm, ³J_{H,H}=9.0 Hz, 2H, 10-CH), 7.72 (d, ³J_{H,H}=7.9 Hz, 1H, 18-CH), 8.49 (d, ³J_{H,H}=3.7 Hz, 1H, 16-CH), 8.56 (d, ⁴J_{H,H}=1.5 Hz, 1H, 15-CH), 8.70 (s, 1H, 19-NH). ¹³C NMR (75 MHz, CDCl₃): δ 26.2 (q, C-7), 33.9 (t, C-3), 45.8 (t, C-13), 55.6 (q, C-12), 57.6 (d, C-2), 82.2 (s, C-6), 114.4 (d, C-9), 119.0 (t, C-5), 123.3 (d, C-17), 129.3 (d, C-10), 131.0 (s, C-8), 132.5 (d, C-4), 132.8 (s, C-14), 136.4 (d, C-18), 148.8 (d, C-16), 149.8 (d, C-15), 163.2 (s, C-11), 167.7 (s, C-1). Elemental analysis: C₂₂H₂₉N₃O₅S (M=447.18 g/mol), calcd. C, 59.04; H, 6.53; N, 9.39. found C, 59.11; H, 6.57; N, 9.10%. Exact mass (ESI⁺): C₂₂H₂₉N₃O₅S+H⁺, calcd. 448.1906. found 448.1907; C₂₂H₂₉N₃O₅S+Na⁺, calcd. 895.3734. found 895.3730; (C₂₂H₂₉N₃O₅S)₂+H⁺, calcd. 470.1726. found 470.1722; (C₂₂H₂₉N₃O₅S)₂+Na⁺, calcd. 917.3554. found 917.3552. MS (ESI⁺, daughter ion experiment): m/z (%) 448 (10) [M⁺+H⁺], 392 (100) [448-C₄H₈], 331 (32) [448-C₅H₁₁NO₂], 279 (8) [331-C₄H₆], 240 (7), 171 (30) [C₇H₇O₃S⁺], 161 (20) [331-C₇H₇O₃S], 125 (11), 107 (2) [C₇H₇O⁺], 92 (8) [C₆H₆N⁺]. Optical rotation: [α]₅₈₉²⁰=−30.4, [α]₅₇₈²⁰=−31.9, [α]₅₄₆²⁰=−37.0, [α]₄₃₆²⁰=−69.1, [α]₃₆₅²⁰=−126.5 (c=0.998, CHCl₃).

### Example 17b

### Tert-butyl (R)—N-(4-methoxyphenylsulfonyl)-N-(3-pyridylmethyl)-2-aminopent-4-enhydroxamate

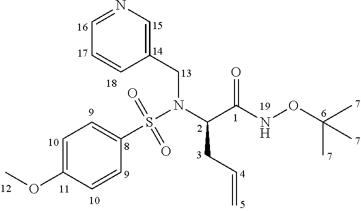

According to the general procedure (R)—N-(4-methoxyphenylsulfonyl)-N-(3-pyridylmethyl)-aminopent-4-enoic acid (150 mg, 0.40 mmol) was reacted with HOBT (54 mg, 0.40 mmol), NMM (202 mg, 1.99 mmol), O-tert-butylhydroxylamine (151 mg 1.20 mmol) and EDC (100 mg 0.52 mmol) in dichloromethane (15 mL). After column chromatography (column Ø 2 cm×15 cm) the product was isolated as white solid. Yield: 130 mg (73%). M.p. 65-66° C. ¹H NMR (300 MHz, CDCl₃): δ 1.21 (s, 9H, 7-CH₃), 2.17 (dt, ²J_{H,H}=14.0 Hz, ³J_{H,H}=6.9 Hz, 1H, 3-CH_{A}), 2.60 (ddd, ²J_{H,H}=14.8 Hz, ³J_{H,H}=8.3 Hz, ³J_{H,H}=6.8 Hz, 1H, 3-CH_{B}), 3.86 (s, 3H, 12-CH₃), 4.31 (t, ³J_{H,H}=7.6 Hz, 1H, 2-CH), 4.60 (s, 2H, 13-CH₂), 4.95 (dd, ³J_{H,H}=13.7 Hz, ²J_{H,H}=1.9 Hz, 2H, 5-CH₂), 5.40 (td, ³J_{H,H}=16.8 Hz, ³J_{H,H}=6.9 Hz, 1H, 4-CH), 6.93 (dm, ³J_{H,H}=8.9 Hz, 2H, 9-CH), 7.23 (dd, ³J_{H,H}=7.8 Hz, ³J_{H,H}=4.9 Hz, 1H, 17-CH), 7.65 (dm, ³J_{H,H}=8.9 Hz, 2H, 10-CH), 7.75 (d, ³J_{H,H}=7.9 Hz, 1H, 18-CH), 8.50 (d, ³J_{H,H}=3.8 Hz, 1H, 16-CH), 8.58 (s, 1H, 15-CH), 8.75 (s, 1H, 19-NH). ¹³C NMR (75 MHz, CDCl₃): δ 26.2 (q, C-7), 34.0 (t, C-3), 45.7 (t, C-13), 55.6 (q, C-12), 57.6 (d, C-2), 82.2 (s, C-6), 114.4 (d, C-9), 118.9 (t, C-5), 123.3 (d, C-17), 129.3 (d, C-10), 131.0 (s, C-8), 132.5 (d, C-4), 133.1 (s, C-14), 136.8 (d, C-18), 148.5 (d, C-16), 149.5 (d, C-15), 163.2 (s, C-11), 167.7 (s, C-1). Exact mass (ESI⁺): C₂₂H₂₉N₃O₅S+Fr, calcd. 448.1906. found 448.1900; C₂₂H₂₉N₃O₅S+Na⁺, calcd. 470.1726. found 470.1722; (C₂₂H₂₉N₃O₅S)₂+H⁺, calcd. 895.3734. found 895.3730; (C₂₂H₂₉N₃O₅S)₂+Na⁺, calcd. 917.3554. found 917.3546. MS (ESI⁺, daughter ion experiment): m/z (%) 448 (50) [M⁺+H⁺], 392 (100) [448-C₄H₈], 331 (13) [448-H⁺-CONHOC(CH₃)₃], 240 (8) [331-C₆H₆N], 171 (12) [C₇H₇O₃S⁺]. Optical rotation: [α]₅₈₉²⁰=+29.6, [α]₅₇₈²⁰=+31.2, [α]₅₄₆²⁰=+36.0, [α]₄₃₆²⁰=+66.9, [α]₃₆₅²⁰=+121.9 (c=1.010, CHCl₃).

### Example 18

### Example 18a

### Tert-butyl (S)—N-(4-methoxyphenylsulfonyl)-N-(3-pyridylmethyl)-2-amino-4-fluorobutanhydroxamate

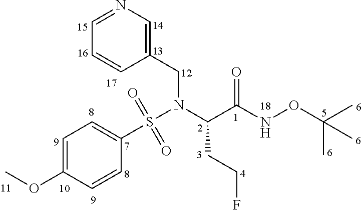

According to the general procedure (S)—N-(4-methoxyphenylsulfonyl)-N-(3-pyridylmethyl)-2-amino-4-fluorobutanoic acid (395 mg, 1.03 mmol) was reacted with HOBT (140 mg, 1.03 mmol), NMM (522 mg, 5.17 mmol), O-tert-butylhydroxylamine (390 mg, 3.10 mmol) and EDC (257 mg, 1.34 mmol) in dichloromethane (15 mL). After column chromatography (column Ø 2 cm×15 cm) the product was isolated as white solid. Yield: 355 mg (75%). M.p. 59-60° C. ¹H NMR (300 MHz, CDCl₃): δ 1.24 (s, 9H, 6-CH₃), 1.72 (m, ³J_{H,F}=22.5 Hz, 1H, 3-CH_{A}), 2.22 (m, ³J_{H,F}=31.6 Hz, 1H 3-CH_{B}), 3.86 (s, 3H, 11-CH₃), 4.25 (m, ²J_{H,F}=47.6 Hz, 2H, 4-CH₂F), 4.49 (d, ³J_{H,H}=16.2 Hz, 1H, 12-CH_{A}), 4.55 (m, 1H, 2-CH), 4.61 (d, ³J_{H,H}=16.0 Hz, 1H, 12-CH_{B}), 6.94 (d, ³J_{H,H}=8.8 Hz, 2H, 8-CH), 7.22 (dd, ³J_{H,H}=7.8 Hz, ³J_{H,H}=4.8 Hz, 1H, 16-CH), 7.62-7.71 (m, ³J_{H,H}=8.9 Hz, 3H, 9-CH & 17-CH), 8.49-8.53 (m, 2H, 14-CH & 15-CH), 8.81 (m, 1H, 18-NH). ¹³C NMR (75 MHz, CDCl₃): δ 26.9 (s, C-6), 30.6 (d, ²J_{H,F}=19.5 Hz, C-3), 45.8 (s, C-12), 53.9 (d, ³J_{H,F}=3.0 Hz, C-2), 55.6 (s, C-11), 80.0 (d, ¹J_{H,F}=165.6 Hz, C-4), 82.4 (s, C-5), 114.5 (s, C-8), 123.4 (s, C-16), 129.2 (s, C-9), 130.8 (s, C-7), 132.6 (s, C-13), 136.4 (s, C-17), 149.1 (s, C-15), 149.8 (s, C-14), 163.3 (s, C-10), 167.4 (s, C-1). ¹⁹F NMR (282 MHz, CDCl₃): δ −222.2 (ddt, ²J_{H,F}=47.5 Hz, ³J_{H,F}=31.5 Hz, ³J_{H,F}=22.6 Hz, 4-CH₂F). Exact mass (ESI⁺): C₂₁H₂₈FN₃O₅S+H⁺, calcd. 454.1812. found 454.1804; C₂₁H₂₈FN₃O₅S+Na⁺, calcd. 476.1631. found 476.1623; (C₂₁H₂₈FN₃O₅S)₂+H⁺, calcd. 907.3546. found 907.3544; (C₂₁H₂₈FN₃O₅S)₂+Na⁺, calcd. 929.3365. found 929.3370. MS (GC/MS, 70 eV): m/z (%) 454 (100) [M⁺+H⁺], 398 (97) [M⁺-C₄H₈], 337 (15) [M⁺-CONHOC(CH₃)₃], 317 (4) [337-HF], 246 (3) [337-C₆H₆N], 171 (15) [C₇H₇O₃S⁺].

Optical rotation: [α]₅₈₉²⁰=+17.9, [α]₅₇₈²⁰=+18.7, [α]₅₄₆²⁰=+21.7, [α]₄₃₆²⁰=+40.7, [α]₃₆₅²⁰=+75.1 (c=1.005, CHCl₃).

### Example 18b

### Tert-butyl (R)—N-(4-methoxyphenylsulfonyl)-N-(3-pyridylmethyl)-2-amino-4-fluorobutanhydroxamate

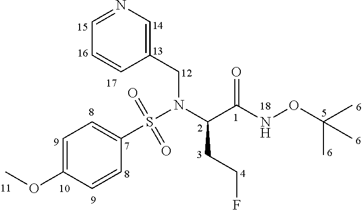

According to the general procedure (R)—N-(4-methoxyphenylsulfonyl)-N-(3-pyridylmethyl)-2-amino-4-fluorobutanoic acid (325 mg, 0.85 mmol) with HOBT (115 mg, 0.85 mmol), NMM (430 mg, 4.25 mmol), O-tert-butylhydroxylamine (320 mg, 2.55 mmol) and EDC (213 mg, 1.11 mmol) in dichloromethane (15 mL). After column chromatography (column Ø 2 cm×10 cm) the product as white solid. Yield: 353 mg (91%). M.p. 60° C. ¹H NMR (400 MHz, CDCl₃): δ 1.24 (s, 9H, 6-CH₃), 1.71 (m, ³J_{H,F}=22.7 Hz, 1H, 3-CH_{A}), 2.22 (m, ³J_{H,F}=28.2 Hz, 1H, 3-CH_{B}), 3.87 (s, 3H, 11-CH₃), 4.17-4.37 (m, ²J_{H,F}=46.9 Hz, 2H, 4-CH₂F), 4.50 (d, ²J_{H,H}=16.0 Hz, 1H, 12-CH_{A}), 4.55 (dd, ³J_{H,H}=8.8 Hz, ³J_{H,H}=5.9 Hz, 1H, 2-CH), 4.61 (d, ²J_{H,H}=16.0 Hz, 1H, 12-CH_{B}), 6.95 (dm, ³J_{H,H}=8.9 Hz, 2H, 8-CH), 7.24 (dd, ³J_{H,H}=7.8 Hz, ³J_{H,H}=4.8 Hz, 1H, 16-CH), 7.66 (dm, ³J_{H,H}=8.9 Hz, 2H, 9-CH), 7.71 (d, ³J_{H,H}=7.9 Hz, 1H, 17-CH), 8.51 (d, ³J_{H,H}=4.9 Hz, ⁴J_{H,H}=1.2 Hz, 1H, 15-CH), 8.53 (d, ⁴J_{H,H}=1.7 Hz, 1H, 14-CH), 8.76 (s, 1H, 18-NH). ¹³C NMR (100 MHz, CDCl₃): δ 26.2 (s, C-6), 30.6 (d, ²J_{H,F}=19.9 Hz, C-3), 55.6 (s, C-11), 80.0 (d, ¹J_{H,F}=165.9 Hz, C-4), 45.8 (s, C-12), 53.9 (d, ³J_{H,F}=2.5 Hz, C-2), 82.4 (s, C-5), 114.5 (s, C-8), 123.4 (s, C-16), 129.3 (s, C-9), 130.8 (s, C-7), 132.7 (s, C-13), 136.6 (s, C-17), 148.8 (s, C-15), 149.6 (s, C-14), 163.4 (s, C-10), 167.4 (s, C-1). ¹⁹F NMR (282 MHz, CDCl₃): δ −221.4 (ddt, ²J_{H,F}=46.9 Hz, ³J_{H,F}=28.4 Hz, ³J_{H,F}=22.8 Hz, 4-CH₂F). Elemental analysis: C₂₁H₃₀FN₃O₅S (M=455.54 g/mol): calcd. C, 55.61, H, 6.22, N, 9.27%. found C, 55.24; H, 6.47; N, 9.22%. Exact mass (ESI⁺): C₂₁H₂₈FN₃O₅S+H⁺, calcd. 454.1812. found 454.1817; C₂₁H₂₈FN₃O₅S+Na⁺, calcd. 476.1631. found 476.1635; (C₂₁H₂₈FN₃O₅S)₂+H⁺, calcd. 907.3546. found 907.3567; (C₂₁H₂₈FN₃O₅S)₂+Na⁺, calcd. 929.3365. found 929.3387. MS (GC/MS, 70 eV): m/z (%) 453 (0) [M⁺], 433 (2) [M⁺—HF], 377 (10) [433-C₄H₈], 347 (13), 277 (4) [M⁺-C₈H₁₅FNO₂], 262 (47) [277-CH₃], 213 (3) [C₉H₁₁NO₃S⁺], 206 (100), 171 (20) [C₇H₇O₃S⁺], 155 (14), 107 (40) [C₇H₇O₃S⁺], 92 (34) [C₆H₆N⁺], 77 (8) [C₆H₅⁺], 57 (15) [C₄H₉⁺], 56 (3) [C₄H₈⁺]. Optical rotation: [α]₅₈₉²⁰=−14.8, [α]₅₇₈²⁰=−15.7, [α]₅₄₆²⁰=+18.1, [α]₄₃₆²⁰=+34.3, [α]₃₆₅²⁰=−62.8 (c=1.004, CHCl₃).

### Example 19

### Example 19a

### Tert-butyl (S)—N-(4-methoxyphenylsulfonyl)-N-(3-pyridylmethyl)-2-amino-4-fluoropent-4-enhydroxamate

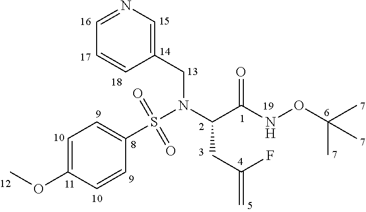

According to the general procedure (S)—N-(4-methoxyphenylsulfonyl)-N-(3-pyridylmethyl)-2-amino-4-fluoropent-4-enoic acid (150 mg, 0.38 mmol) with HOBT (52 mg, 0.38 mmol), NMM (193 mg, 1.90 mmol), O-tert-butylhydroxylamine (144 mg, 1.14 mmol) and EDC (95 mg, 0.50 mmol) in dichloromethane (15 mL). After column chromatography (column: Ø 2 cm×10 cm) was isolated as white solid. Yield: 107 mg (61%). M.p. 60-61° C. ¹H NMR (300 MHz, CDCl₃): δ 1.23 (s, 9H, 7-CH₃), 2.26 (ddd, ³J_{H,F}=17.5 Hz, ²J_{H,H}=14.7 Hz, ³J_{H,H}=6.2 Hz, 1H, 3-CH_{A}), 2.80 (ddd, ³J_{H,F}=19.6 Hz, ²J_{H,H}=14.7 Hz, ³J_{H,H}=8.2 Hz, 1H, 3-CH_{B}), 3.86 (s, 3H, 12-CH₃), 4.15 (dd, ³J_{H,F}=49.7 Hz, ²J_{H,H}=3.1 Hz, 1H, 5-CHₜᵣₐₙₛ 4.48 (dd, ³J_{H,F}=17.1 Hz, ²J_{H,H}=3.1 Hz, 1H, 5-CH_{cis}), 4.50 (d, ²J_{H,H}=16.2 Hz, 1H, 13-CH_{A}), 4.59 (m, 1H, 2-CH), 4.62 (d, ²J_{H,H}=16.0 Hz, 1H, 13-CH_{B}), 6.93 (d, ³J_{H,H}=8.9 Hz, 2H, 9-CH), 7.20 (dd, ³, J_{H,H}=7.8 Hz, ³J_{H,H}=4.8 Hz, 1H, 17-CH), 7.67 (t, ³J_{H,H}=8.9 Hz, 3H, 10-CH and 18-CH), 8.49 (d, ³J_{H,H}=3.8 Hz, 1H, 16-CH), 8.53 (d, ⁴J_{H,H}=1.7 Hz, 1H, 15-CH), 8.69 (s, 1H, 19-NH). ¹³C NMR (75.48 MHz, CDCl₃): δ 26.2 (q, C-7), 32.8 (dt, ²J_{H,F}=27.9 Hz, C-3), 45.9 (t, C-13), 54.9 (d, C-2), 55.7 (q, C-12), 82.5 (s, C-6), 93.9 (dt, ²J_{H,F}=18.5 Hz, C-5), 114.4 (d, C-9), 123.3 (d, C-17), 129.5 (d, C-10), 130.9 (s, C-8), 132.5 (s, C-14), 136.4 (d, C-18), 149.1 (d, C-16), 145.0 (d, C-15), 161.1 (ds, ¹J_{H,F}=256.4 Hz, C-4), 163.4 (s, C-11), 166.9 (s, C-1). ¹⁹F NMR (282 MHz, CDCl₃): δ −98.4 (ddd, ³J_{H,F}=49.6 Hz, ³J_{H,F}=17.9 Hz, ³J_{H,F}=17.6 Hz, 4-CF). Exact mass (ESI⁺): C₂₂H₂₈FN₃O₅S+H⁺, calcd. 466.1812. found 466.1819; C₂₂H₂₈FN₃O₅S+Na⁺, calcd. 488.1631. found 488.1634; (C₂₂H₂₈FN₃O₅S)₂+H⁺, calcd. 931.3546. found 931.3565; (C₂₂H₂₈FN₃O₅S)₂+Na⁺, calcd. 953.3384. found 953.3384. MS (ESI⁺, daughter ion experiment): m/z (%) 466 (18) [M⁺+H⁺], 410 (100) [466-C₄H₈], 349 (13) [466-H⁺-CONHOC(CH₃)₃], 179 (11) [410-C₇H₇O₃S⁺—C₃H₄F], 171 (30) [C₇H₇O₃S⁺], [α]₅₈₉²⁰=−6.5, [α]₅₇₈²⁰=−6.8, [α]₅₄₆²⁰=−7.9, [α]₄₃₆²⁰=−14.3, [α]₃₆₅²⁰=−24.9 (c=0.996, CHCl₃).

### Example 19b

### Tert-butyl (R)—N-(4-methoxyphenylsulfonyl)-N-(3-pyridylmethyl)-2-amino-4-fluoropent-4-enhydroxamate

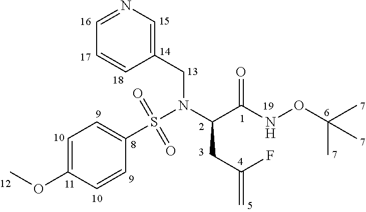

According to the general procedure (R)—N-(4-methoxyphenylsulfonyl)-N-(3-pyridylmethyl)-2-amino-4-fluoropent-4-enoic acid (150 mg, 0.38 mmol) was reacted with HOBT (52 mg, 0.38 mmol), NMM (193 mg, 1.90 mmol), O-tert-butylhydroxylamine (144 mg, 1.14 mmol) and EDC (95 mg, 0.50 mmol) in dichloromethane (15 mL). After column chromatography (column: Ø 2 cm×10 cm) the product was isolated as white solid. Yield: 112 mg (63%). M.p. 59-60° C. ¹H NMR (300 MHz, CDCl₃): δ 1.24 (s, 9H, 7-CH₃), 2.27 (m, 1H, 3-CH_{A}), 2.79 (ddd, ³J_{H,F}=19.8 Hz, ²J_{H,H}=14.7 Hz, ³J_{H,H}=8.3 Hz, 1H, 3-CH_{B}), 3.86 (s, 3H, 12-CH₃), 4.15 (dd, ³J_{H,F}=49.8 Hz, ²J_{H,H}=3.1 Hz, 1H, 5-CHₜᵣₐₙₛ), 4.48 (dd, ³J_{H,F}=17.0 Hz, ²J_{H,H}=3.2 Hz, 1H, 5-CH_{cis}), 4.50 (d, ²J_{H,H}=16.1 Hz, 1H, 13-CH_{A}), 4.56-4.66 (m, 2H, 2-CH and 13-CH_{B}), 6.93 (d, ³J_{H,H}=9.0 Hz, 2H, 9-CH), 7.21 (dd, ³J_{H,H}=7.8 Hz, ³J_{H,H}=4.9 Hz, 1 H, 17-CH), 7.62-7.73 (m, 3H, 10-CH & 18-CH), 8.50 (dd, ³J_{H,H}=5.3 Hz, ²J_{H,H}=1.3 Hz, 1 H, 16-CH), 8.53 (d, ²J_{H,H}=1.3 Hz, 1H, 15-CH), 8.72 (s, 1H, 19-NH). ¹³C NMR (75 MHz, CDCl₃): δ 26.2 (q, C-7), 32.8 (dt, ²J_{H,F}=27.7 Hz, C-3), 45.8 (t, C-13), 54.8 (d, C-2), 55.7 (q, C-12), 82.5 (s, C-6), 94.0 (dt, ²J_{H,F}=18.2 Hz, C-5), 114.4 (d, C-9), 123.3 (d, C-17), 129.5 (d, C-10), 130.8 (s, C-8), 132.5 (s, C-14), 136.5 (d, C-18), 149.0 (d, C-16), 149.9 (d, C-15), 161.0 (ds, ¹J_{H,F}=256.9 Hz, C-4), 163.4 (s, C-11), 166.9 (s, C-1). ¹⁹F NMR (282 MHz, CDCl₃): δ −98.4 (ddd, ³J_{H,F}=49.8 Hz, ³J_{H,F}=19.1 Hz, ³J_{H,F}=17.4 Hz, 4-CF). Exact mass (ESI⁺): C₂₂H₂₈FN₃O₅S+H⁺, calcd. 466.1812. found 466.1806; C₂₂H₂₈FN₃O₅S+Na⁺, calcd. 488.1631. found 488.1626; (C₂₂H₂₈FN₃O₅S)₂+H⁺, calcd. 931.3546. found 931.3519; MS (ESI⁺, daughter ion experiment): m/z (%) 466 (40) [M⁺+H⁺], 410 (100) [466-C₄H₈], 349 (10) [466-H⁺—CONHOC(CH₃)₃], 171 (5) [C₇H₇O₃S⁺]. Optical rotation: [α]₅₈₉²⁰=+8.5, [α]₅₇₈²⁰=+8.7, [α]₅₄₆²⁰=+10.1, [α]₄₃₆²⁰=+18.2, [α]₃₆₅²⁰=+31.8 (c=1.023, CHCl₃).

General Procedure for the Hydrolysis of Hydroxamates to Hydroxamic Acids

In a dried YOUNG-tube, the corresponding hydroxamate is dissolved in fresh trifluoroacetic acid (50 mL/mmol) under argon. The YOUNG-tube is flashed with argon and closed. The solution is stirred at 40° C. for 12 h. Then the reaction mixture is evaporated to dryness in vacuum. The residue is dissolved in chloroform (100 mL/mmol) and washed with aqueous citric acid (pH 4) (25 mL/mmol) and bicarbonate. The aqueous phase is extracted with chloroform and the combined organic phases are dried with magnesium sulfate. After evaporation of the solvent the product is isolated as viscos oil.

### Example 20

### Example 20a (S)—N-(4-Methoxyphenylsulfonyl)-N-(3-pyridylmethyl)-2-amino-butanhydroxamic acid

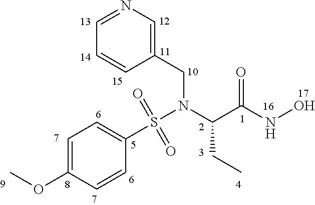

According to the general procedure tert-butyl (S)—N-(4-methoxyphenyl-sulfonyl)-N-(3-pyridylmethyl)-2-aminobutanhydroxamate (173 mg, 0.40 mmol) was hydrolyzed in trifluoroacetic acid (25 mL). After work up, the product was isolated as a cloudy, highly viscos liquid. After purification with preparative HPLC the product was 96% pure. Yield: 60 mg (40%). ¹H NMR (300.13 MHz, CD₃CN): δ 0.71 (t, ³J_{H,H}=7.4 Hz, 3H, 4-CH₃), 1.40 (m, 1H, 3-CH_{A}), 1.66 (m, 1H, 3-CH_{B}), 3.86 (s, 3H, 9-CH₃), 4.18 (dd, ³J_{H,H}=8.7 Hz, ³J_{H,H}=6.6 Hz, 1H, 2-CH), 4.74 (d, ²J_{H,H}=17.4 Hz, 1H, 10-CH_{A}), 4.86 (d, ²J_{H,H}=17.4 Hz, 1H, 10-CH_{B}), 7.03 (d, ³J_{H,H}=9.0 Hz, 2H, 6-CH), 7.71 (d, ³J_{H,H}=9.1 Hz, 2H, 7-CH), 7.91 (dd, ³J_{H,H}=8.1 Hz, ³J_{H,H}=5.8 Hz, 1H, 14-CH), 8.50 (d, ³J_{H,H}=8.1 Hz, 1H, 15-CH), 8.58 (d, ³J_{H,H}=5.7 Hz, 1H, 13-CH), 8.77 (s, 1H, 12-CH). ¹³C NMR (75.48 MHz, CD₃CN): 10.7 (q, C-4), 23.8 (t, C-3), 46.0 (t, C-10), 56.7 (q, C-9), 59.7 (d, C-2), 115.6 (d, C-6), 127.7 (d, C-14), 130.5 (d, C-7), 131.6 (s, C-5), 141.0 (d, C-12), 141.1 (s, C-11), 142.1 (d, C-13), 146.4 (d, C-15), 164.6 (s, C-9), 168.3 (s, C-1). Exact mass (ESI⁺): C₁₇H₂₁N₃O₅S+H⁺, calcd. 380.1280. found 380.1263; C₁₇H₂₁N₃O₅S+Na⁺, calcd. 402.1100. found 402.1082. MS (ESI⁺, daughter ion experiment): m/z (%) 380 (100) [M⁺+H⁺], 319 (25) [M⁺-CONHOH], 228 (5) [319-C₆H₆N], 171 (30) [C₇H₇O₃S⁺], 92 (10) [C₆H₆N⁺].

### Example 20b

### (R)—N-(4-Methoxyphenylsulfonyl)-N-(3-pyridylmethyl)-2-amino-butanhydroxamic acid

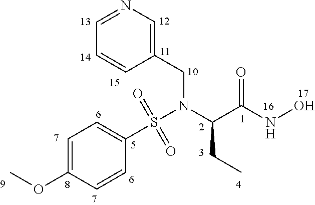

According to the general procedure tert-butyl (R)—N-(4-methoxyphenyl-sulfonyl)-N-(3-pyridylmethyl)-2-aminobutanhydroxamate (101 mg, 0.23 mmol) was hydrolyzed in trifluoroacetic acid (25 mL). After workup, the product was isolated as white, highly viscos liquid. HPLC showed 95% purity of the product. Yield: 38 mg (43%). ¹H NMR (300 MHz, CD₃CN): δ 0.72 (t, ³J_{H,H}=7.4 Hz, 3H, 4-CH₃), 1.40 (m, 1H, 3-CH_{A}), 1.76 (m, 1H, 3-CH_{B}), 3.85 (s, 3H, 9-CH₃), 4.17 (dd, ³J_{H,H}=8.7 Hz, ³J_{H,H}=6.6 Hz, 1H, 2-CH), 4.75 (d, ²J_{H,H}=17.4 Hz, 1H, 10-CH_{A}), 4.83 (d, ²J_{H,H}=17.3 Hz, 1H, 10-CH_{B}), 7.01 (d, ³J_{H,H}=9.0 Hz, 2H, 6-CH), 7.69 (d, ³J_{H,H}=9.0 Hz, 2H, 7-CH), 7.88 (dd, ³J_{H,H}=8.0 Hz, ³J_{H,H}=5.8 Hz, 1H, 14-CH), 8.44 (d, ³J_{H,H}=8.1 Hz, 1H, 15-CH), 8.59 (d, ³J_{H,H}=5.5 Hz, 1H, 13-CH), 8.77 (s, 1H, 12-CH). ¹³C NMR (75 MHz, CD₃CN): δ 10.6 (q, C-4), 23.7 (t, C-3), 46.9 (t, C-10), 56.6 (q, C-9), 59.7 (d, C-2), 115.6 (d, C-6), 127.5 (d, C-14), 130.4 (d, C-7), 131.7 (s, C-5), 140.8 (d, C-12), 141.1 (s, C-11), 142.4 (d, C-13), 146.0 (d, C-15), 164.6 (s, C-9), 168.2 (s, C-1). Exact mass (ESI⁺): C₁₇H₂₁N₃O₅S+Na⁺, calcd. 380.1280. found 380.1278; C₁₇H₂₁N₃O₅S+Na⁺, calcd. 402.1100. found 402.1094. MS (ESI⁺, daughter ion experiment): m/z (%) 759 (100) [2M⁺+H⁺], 380 (18) [M⁺+H⁺], 279 (13), 227 (13) [C₁₀H₁₃NO₃S⁺], 155 (8) [C₇H₇O₂S⁺].

### Example 21

### Example 21a

### (S)—N-(4-Methoxyphenylsulfonyl)-N-(3-pyridylmethyl)-2-aminopent-4-enhydroxamic acid

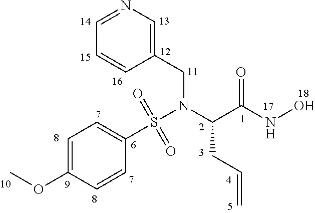

According to the general procedure tert-butyl (S)—N-(4-methoxyphenyl-sulfonyl)-N-(3-pyridylmethyl)-2-aminopent-4-enhydroxamate (300 mg, 0.67 mmol) was hydrolyzed in trifluoroacetic acid (25 mL). After workup, the product was isolated as white, highly viscos liquid. HPLC showed 96% purity of the product. Yield: 77 mg (30%). ¹H NMR (400 MHz, CD₃CN): δ 2.14 (dt, ²J_{H,H}=14.3 Hz, ³J_{H,H}=6.8 Hz, 1H, 3-CH_{A}), 2.45 (dt, ²J_{H,H}=14.2 Hz, ³J_{H,H}=7.7 Hz, 1H, 3-CH_{B}), 3.84 (s, 3H, 10-CH₃), 4.31 (t, ³J_{H,H}=7.6 Hz, 1H, 2-CH), 4.61 (d, ²J_{H,H}=16.8 Hz, 1H, 11-CH_{A}), 4.73 (d, ²J_{H,H}=16.8 Hz, 1H, 11-CH_{B}), 4.92 (s, 1H, 5-CH_{A}), 4.95 (d, ³J_{H,H}=5.6 Hz, 1H, 5-CH_{B}), 5.47 (ddt, ³J_{H,H}=17.3 Hz, ³J_{H,H}=10.4 Hz, ³J_{H,H}=6.9 Hz, 1H, 4-CH), 7.00 (d, ³J_{H,H}=8.8 Hz, 2H, 7-CH), 7.30 (dd, ³J_{H,H}=7.6 Hz, ³J_{H,H}=4.9 Hz, 1H, 15-CH), 7.70 (d, ³J_{H,H}=8.9 Hz, 2H, 8-CH), 7.81 (d, ³J_{H,H}=7.8 Hz, 1H, 16-CH), 8.41 (s, 1H, 14-CH), 8.53 (s, 1H, 13-CH). ¹³C NMR (100 MHz, CD₃CN): δ 35.2 (t, C-3), 46.8 (t, C-11), 56.6 (q, C-10), 58.0 (d, C-2), 115.4 (d, C-7), 119.1 (t, C-5), 124.5 (d, C-15), 130.5 (d, C-8), 132.1 (d, C-4), 133.9 (s, C-12), 135.9 (s, C-6), 137.7 (d, C-16), 148.5 (d, C-14), 149.5 (d, C-13), 164.4 (s, C-9), 167.4 (s, C-1). Exat mass (ESI⁺): C₁₈H₂₁N₃O₅S+H⁺, calcd. 392.1280. found 392.1271; C₁₈H₂₁N₃O₅S+Na⁺, calcd. 414.1100. found 414.1094. MS (ESI⁺, daughter ion experiment): m/z (%) 392 (10) [M⁺+H⁺], 331 (15) [M⁺-CONHOH], 279 (4) [C₁₃H₁₅N₂O₃S⁺], 240 (8) [331-C₅H₄N], 171 (100) [C₇H₇O₃S⁺], 161 (30) [C₁₀H₁₃N₂⁺], 92 (45) [C₆H₆N⁺].

### Example 21b

### (R)—N-(4-Methoxyphenylsulfonyl)-N-(3-pyridylmethyl)-2-aminopent-4-enhydroxamic acid

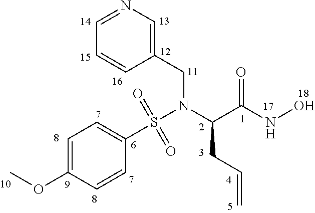

According to the general procedure tert-butyl (R)—N-(4-methoxyphenyl-sulfonyl)-N-(3-pyridylmethyl)-2-aminopent-4-enhydroxamate (100 mg, 0.22 mmol) was hydrolyzed in trifluoroacetic acid (25 mL). After workup, the product was isolated as white, highly viscos liquid. HPLC showed 97% purity of the product. Yield: 33 mg (38%). ¹H NMR (400 MHz, CD₃CN): δ 2.15 (dt, ²J_{H,H}=14.1 Hz, ³J_{H,H}=6.9 Hz, 1H, 3-CH_{A}), 2.46 (dt, ²J_{H,H}=14.1 Hz, ³, J_{H,H}=7.6 Hz, 1H, 3-CH_{B}), 3.84 (s, 3H, 10-CH₃), 4.29 (t, ³J_{H,H}=7.6 Hz, 1H, 2-CH), 4.60 (d, ²J_{H,H}=16.7 Hz, 1H, 11-CH_{A}), 4.72 (d, ²J_{H,H}=16.7 Hz, 1H, 11-CH_{B}), 4.93 (s, 1H, 5-CH_{A}), 4.96 (d, ³J_{H,H}=4.5 Hz, 1H, 5-CH_{B}), 5.48 (ddt, ³J_{H,H}=16.8 Hz, ³J_{H,H}=9.6 Hz, ³J_{H,H}=6.9 Hz, 1H, 4-CH), 7.00 (d, ³J_{H,H}=8.7 Hz, 2H, 7-CH), 7.29 (dd, ³J_{H,H}=7.8 Hz, ³J_{H,H}=4.8 Hz, 1H, 15-CH), 7.70 (d, ³J_{H,H}=8.9 Hz, 2H, 8-CH), 7.79 (m, 1H, 16-CH), 8.41 (d, ³J_{H,H}=4.4 Hz, 1H, 14-CH), 8.52 (s, 1H, 13-CH). ¹³C NMR (100 MHz, CD₃CN): 35.2 (t, C-3), 46.8 (t, C-11), 56.6 (q, C-10), 58.0 (d, C-2), 115.4 (d, C-7), 119.1 (t, C-5), 124.5 (d, C-15), 130.5 (d, C-8), 132.1 (d, C-4), 134.0 (s, C-12), 135.8 (s, C-6), 137.5 (d, C-16), 148.6 (d, C-14), 149.7 (d, C-13), 164.4 (s, C-9), 167.3 (s, C-1). Exact mass (ESI⁺): C₁₈H₂₁N₃O₅S+H⁺, calcd. 392.1280. found 392.1275. MS (ESI⁺, daughter ion experiment): m/z (%) 392 (30) [M⁺ +H⁺], 331 (35) [M⁺-CONHOH], 279 (8) [C₁₃H₁₅N₂O₃S⁺], 240 (20) [331-C₅H₄N], 171 (100) [C₇H₇O₃S⁺], 161 (55) [C₁₀H₁₃N₂⁺], 124 (27) [C₇H₈O₂⁺], 107 (11) [C₇H₇O⁺], 92 (42) [C₆H₆N⁺].

### Example 22

### Example 22a

### (S)—N-(4-Methoxyphenylsulfonyl)-N-(3-pyridylmethyl)-2-amino-4-fluorobutanhydroxamic acid ((S)-155)

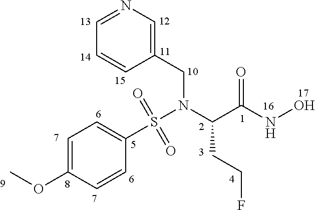

According to the general procedure tert-butyl (S)—N-(4-methoxyphenyl-sulfonyl)-N-(3-pyridylmethyl)-2-amino-4-fluorobutanhydroxamate (131 mg, 0.29 mmol) was hydrolyzed in trifluoroacetic acid (25 mL). After workup, the product was isolated as white, highly viscos liquid. Purification by preparative HPLC delivered a product of 95% purity. Yield: 49 mg (42%). ¹H NMR (600 MHz, CD₃CN): δ 1.76 (m, ³J_{H,F}=23.7 Hz, 1H, 3-CH_{A}), 2.14 (ddd, ³J_{H,F}=29.1 Hz, ²J_{H,H}=11.8 Hz, ³J_{H,H}=6.1 Hz, 1H, 3-CH_{B}), 3.85 (s, 3H, 9-CH₃), 4.20-4.42 (m, 2H, 4-CH₂F), 4.48 (dd, ³J_{H,H}=8.9 Hz, ³J_{H,H}=6.1 Hz, 1H, 2-CH), 4.76 (d, ²J_{H,H}=17.1 Hz, 1H, 10-CH_{A}), 4.81 (d, ²J_{H,H}=17.1 Hz, 1H, 10-CH_{B}), 7.01 (d, ³J_{H,H}=8.9 Hz, 2H, 6-CH), 7.68 (d, ³J_{H,H}=8.9 Hz, 2H, 7-CH), 7.88 (dd, ³J_{H,H}=8.1 Hz, ³J_{H,H}=5.7 Hz, 1H, 14-CH), 8.43 (d, ³J_{H,H}=7.7 Hz, 1H, 15-CH), 8.58 (d, ³J_{H,H}=5.3 Hz, 1H, 12-CH), 8.74 (s, 1H, 13-CH). ¹³C NMR (150 MHz, CD₃CN): δ 31.3 (dt, ²J_{H,F}=20.0 Hz, C-3), 46.1 (t, C-10), 55.0 (dd, ³J_{H,F}=3.6 Hz, C-2), 56.78 (q, C-9), 81.4 (dt, ¹J_{H,F}=164.2 Hz, C-4), 115.7 (d, C-6), 127.6 (d, C-14), 130.2 (d, C-7), 131.4 (s, C-5), 140.4 (s, C-11), 141.2 (d, C-12), 142.3 (d, C-13), 146.1 (d, C-15), 164.7 (s, C-8), 167.3 (s, C-1). ¹⁹F NMR (564 MHz, CD₃CN): δ −221.4 (tdd, ²J_{H,F}=47.1 Hz, ³J_{H,F}=29.0 Hz, ³J_{H,F}=23.7 Hz, 4-CH₂F). Exact mass (ESI⁺): C₁₇H₂₀FN₃O₅S⁺+H⁺, calcd. 398.1186. found 398.1181. C₁₇H₂₀FN₃O₅S⁺+Na⁺, calcd. 420.1105. found 420.0999; (C₁₇H₂₀FN₃O₅S⁺)₂+H⁺, calcd. 795.2294. found 795.2300; (C₁₇H₂₀FN₃O₅S⁺)₂+Na⁺, calcd. 817.2113. found 817.2116. MS (ESI⁺, daughter ion experiment): m/z (%) 398 (10) [M⁺+H⁺], 337 (16) [M⁺-CONHOH], 171 (100) [C₇H₇O₃S⁺], 167 (30) [337-C₇H₇O₃S], 135 (16) [C₈H₁₁N₂⁺], 123 (22) [C₇H₇O₂⁺], 107 (5) [C₇H₇O⁺], 92 (52) [C₆H₆N⁺].

### Example 22b

### (R)—N-(4-Methoxyphenylsulfonyl)-N-(3-pyridylmethyl)-2-amino-4-fluorobutanhydroxamic acid

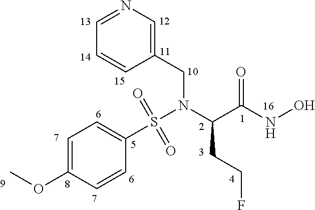

According to the general procedure tert-butyl (R)—N-(4-methoxyphenyl-sulfonyl)-N-(3-pyridylmethyl)-2-amino-4-fluorobutanhydroxamate (104 mg, 0.23 mmol) was hydrolyzed in trifluoroacetic acid (25 mL). After workup, the product was isolated as white, highly viscos liquid. Purification by preparative HPLC delivered a product of 95% purity. Yield: 45 mg (49%). ¹H NMR (400 MHz, CD₃CN): δ 1.67 (m, 1H, 3-CH_{A}), 2.13 (m, 1H, 3-CH_{B}), 3.86 (s, 3H, 9-CH₃), 4.17-4.46 (m, 2H, 4-CH₂F), 4.49 (dd, ³J_{H,H}=8.8 Hz, ³J_{H,H}=6.1 Hz, 1H, 2-CH), 4.75 (d, ²J_{H,H}=17.3 Hz, 1H, 10-CH_{A}), 4.86 (d, ²J_{H,H}=17.3 Hz, 1H, 10-CH_{B}), 7.04 (d, ³J_{H,H}=9.0 Hz, 2H, 6-CH), 7.71 (d, ³J_{H,H}=9.0 Hz, 2H, 7-CH), 7.92 (dd, ³J_{H,H}=8.0 Hz, ³J_{H,H}=5.9 Hz, 1H, 14-CH), 8.50 (d, ³J_{H,H}=8.2 Hz, 1H, 15-CH), 8.59 (d, ³J_{H,H}=5.6 Hz, 1H, 13-CH), 8.75 (s, 1H, 12-CH). ¹³C NMR (100 MHz, CD₃CN): δ [ppm] 31.5 (dt, ²J_{H,F}=20.1 Hz, C-3), 46.2 (t, C-10), 55.0 (dd, ³J_{H,F}=3.4 Hz, C-2), 56.7 (q, C-9), 81.3 (dt, ¹J_{H,F}=164.2 Hz, C-4), 115.7 (d, C-6), 127.9 (d, C-14), 130.6 (d, C-7), 131.3 (s, C-5), 140.8 (s, C-11), 141.2 (s, C-13), 142.1 (s, C-12), 146.7 (s, C-15), 164.8 (s, C-8), 167.3 (s, C-1). ¹⁹F NMR (282 MHz, CD₃CN): δ [ppm]-222.2 (tdd, ²J_{H,F}=47.2 Hz, ³J_{H,F}=31.5 Hz, ³J_{H,F}=22.3 Hz, 4-CH₂F). Exact mass (ESI⁺): C₁₇H₂₀N₃O₅S+Fr, calcd. 398.1186. found 398.1171; C₁₇H₂₀N₃O₅S+Na⁺, calcd. 420.1005. found 420.0990; (C₁₇H₂₀N₃O₅S)₂+H⁺, calcd. 795.2294. found 871.3691; (C₁₇H₂₀N₃O₅S)₂+Na⁺, calcd. 817.2113. found 893.3507. MS (ESI⁺, daughter ion experiment): m/z (%) 398 (60) [M⁺+H⁺], 337 (100) [M⁺-CONHOH], 317 (7) [337-HF], 246 (6), 171 (20) [C₇H₇O₃S⁺].

### Example 23

### Example 23a

### (S)—N-(4-Methoxyphenylsulfonyl)-N-(3-pyridylmethyl)-2-amino-4-fluoropent-4-enhydroxamic acid

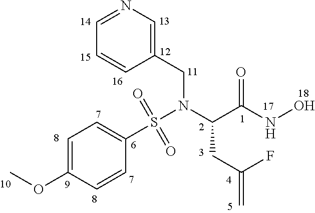

According to the general procedure tert-butyl (S)—N-(4-methoxyphenyl-sulfonyl)-N-(3-pyridylmethyl)-2-amino-4-fluoropent-4-enhydroxamate (107 mg, 0.23 mmol) was hydrolyzed in trifluoroacetic acid (25 mL). After workup, the product was isolated as white, highly viscos liquid. Purification by preparative HPLC delivered a product of 97% purity. Yield: 62 mg (66%). ¹H NMR (400 MHz, CD₃CN): δ 2.35 (ddd, ³J_{H,F}=18.1 Hz, ²J_{H,H}=14.9 Hz, ³J_{H,H}=6.6 Hz, 1H, 3-CH_{A}), 2.74 (ddd, ³J_{H,F}=18.9 Hz, ²J_{H,H}=14.8 Hz, ³J_{H,H}=8.4 Hz, 1H, 3-CH_{B}), 3.85 (s, 3H, 10-CH₃), 4.34 (dd, ³J_{H,F}=50.3 Hz, ²J_{H,H}=3.3 Hz, 1H, 5-CHₜᵣₐₙₛ), 4.56 (dd, ³J_{H,F}=17.3 Hz, ²J_{H,H}=3.3 Hz, 1H, 5-CH_{cis}), 4.57 (dd, ³J_{H,H}=8.2 Hz, ³J_{H,H}=6.5 Hz, 1H, 2-CH), 4.81 (s, 2H, 11-CH₂), 7.01 (d, ³J_{H,H}=9.0 Hz, 2H, 7-CH), 7.66 (d, ³J_{H,H}=9.0 Hz, 2H, 8-CH), 7.86 (dd, ³J_{H,H}=8.0 Hz, ³J_{H,H}=5.8 Hz, 1H, 15-CH), 8.41 (d, ³J_{H,H}=8.2 Hz, 1H, 16-CH), 8.59 (d, ³J_{H,H}=5.5 Hz, 1H, 14-CH), 8.73 (s, 1H, 13-CH). ¹³C NMR (100 MHz, CD₃CN): δ 33.2 (dt, ²J_{H,F}=28.0 Hz, C-3), 46.1 (t, C-11), 55.5 (d, C-2), 56.7 (q, C-10), 94.5 (dt, ²J_{H,F}=18.7 Hz, C-5), 115.7 (d, C-7), 127.5 (d, C-15), 130.5 (d, C-8), 131.5 (s, C-6), 140.1 (s, C-12), 141.4 (d, C-14), 142.6 (d, C-13), 145.7 (d, C-16), 162.5 (ds, ¹J_{H,F}=255.6 Hz, C-4), 164.7 (s, C-9), 166.8 (s, C-1). ¹⁹F NMR (282 MHz, CD₃CN): δ −97.3 (ddd, ³J_{H,F}=50.2 Hz, ³J_{H,F}=18.2 Hz, ³J_{H,F}=17.7 Hz, 4-CF). Exact mass (ESI⁺): C₁₈H₂₀FN₃O₅S⁺+H⁺, calcd. 410.1186. found 410.1174; (C₁₈H₂₀FN₃O₅S⁺)₂+H⁺, caldc. 819.2294. found 819.2278; (C₁₈H₂₀FN₃O₅S⁺)₂+Na⁺, calcd. 841.2113. found 841.2096. MS (ESI⁺, daughter ion experiment): m/z (%) 410 (5) [M⁺+H⁺], 349 (11) [M⁺-CONHOH], 179 (23) [349-C₇H₇O₃S⁻], 171 (100) [C₇H₇O₃S⁺], 135 (18) [C₈H₁₁N₂⁺], 123 (20) [C₇H₇O₂], 92 (50) [C₆H₆N⁺].

### Example 23b

### (R)—N-(4-Methoxyphenylsulfonyl)-N-(3-pyridylmethyl)-2-amino-4-fluoropent-4-enhydroxamic acid

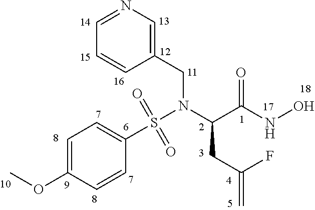

According to the general procedure tert-butyl (R)—N-(4-methoxyphenylsulfonyl)-N-(3-pyridylmethyl)-2-amino-4-fluoropent-4-enhydroxamate (112 mg, 0.24 mmol) was hydrolyzed in trifluoroacetic acid (25 mL). After workup, the product was isolated as white, highly viscos liquid. Purification by preparative HPLC delivered a product of 97% purity. Yield: 57 mg (58%): ¹H NMR (300 MHz, CD₃CN): δ 2.35 (ddd, ³J_{H,F}=18.1 Hz, ²J_{H,H}=14.9 Hz, ³J_{H,H}=6.7 Hz, 1H, 3-CH_{A}), 2.74 (ddd, ³J_{H,F}=18.6 Hz, ²J_{H,H}=14.8 Hz, ³J_{H,H}=8.3 Hz, 1H, 3-CH_{B}), 3.86 (s, 3H, 10-CH₃), 4.34 (dd, ³J_{H,F}=50.3 Hz, ²J_{H,H}=3.2 Hz, 1H, 5-CHₜᵣₐₙₛ), 4.56 (dd, ³J_{H,F}=17.3 Hz, ²J_{H,H}=3.2 Hz, 1H, 5-CH_{cis}), 4.59 (t, ³J_{H,H}=7.5 Hz, 1H, 2-CH), 4.79 (d, ²J_{H,H}=17.5 Hz, 1H, 11-CH_{A}), 4.86 (d, ²J_{H,H}=17.5 Hz, 1H, 11-CH_{B}), 7.01 (d, ³J_{H,H}=9.0 Hz, 2H, 7-CH), 7.68 (d, ³J_{H,H}=9.0 Hz, 2H, 8-CH), 7.89 (dd, ³J_{H,H}=8.0 Hz, ³J_{H,H}=5.8 Hz, 1H, 15-CH), 8.45 (d, ³J_{H,H}=8.1 Hz, 1H, 16-CH), 8.59 (d, ³J_{H,H}=5.5 Hz, 1H, 14-CH), 8.74 (s, 1H, 13-CH). ¹³C NMR (75 MHz, CD₃CN): δ 33.2 (dt, ²J_{H,F}=27.9 Hz, C-3), 46.1 (t, C-11), 55.4 (d, C-2), 56.7 (q, C-10), 94.5 (dt, ²J_{H,F}=18.6 Hz, C-5), 115.7 (d, C-7), 127.6 (d, C-15), 130.6 (d, C-8), 131.3 (s, C-6), 140.4 (s, C-12), 141.2 (d, C-14), 142.2 (d, C-13), 146.1 (d, C-16), 162.4 (ds, ¹J_{H,F}=255.5 Hz, C-4), 164.7 (s, C-9), 166.8 (s, C-1). ¹⁹F NMR (282 MHz, CD₃CN): δ −97.3 (ddd, ³J_{H,F}=50.3 Hz, ³J_{H,F}=18.2 Hz, ³J_{H,F}=17.5 Hz, 4-CF). Exact mass (ESI⁺): C₁₈H₂₀FN₃O₅S⁺+H⁺, calcd. 410.1186. found 410.1174; C₁₈H₂₀FN₃O₅S⁺+Na⁺, calcd. 432.1005. found 432.0991; (C₁₈H₂₀FN₃O₅S⁺)₂+H⁺: calcd. 819.2294. found 819.2278; (C₁₈H₂₀FN₃O₅S⁺)₂+Na⁺, calcd. 841.2113. found 841.2103. MS (ESI⁺, daughter ion experiment): m/z (%) −410 (6) [M⁺+H⁺], 349 (10) [M⁺-CONHOH], 179 (22) [349-C₇H₇O₃S⁺], 171 (100) [C₇H₇O₃S⁺], 135 (18) [C₈H₁₁N₂⁺], 123 (22) [C₇H₇O₂⁺], 92 (50) [C₆H₆N⁺].

General Procedure for Substitution with p-(2-halogenethoxy)-phenylsulfonyl chloride

The tert-butyl 4-fluoro-2-aminopent-4-enecarboxylate is dissolved in pyridine and treated with 1 equivalent of p-(2-halogenethoxy)phenylsulfonyl chloride under stiffing at 0° C. The reaction mixture is allowed to warm up to r.t. and stirred for 40 h. Then the mixture is diluted with dichloromethane and the organic solution is washed with 0.5 N HCl, water (2 times each) and saturated aqueous sodium chloride. After drying with magnesium sulfate the solvent was removed in vacuum. The product is passed through a short pad of silica gel and recrystallized from ethyl acetate/cyclohexane. The products are isolated as white crystalline solids.

### Example 24

### Example 24a

### Compound MAB 239

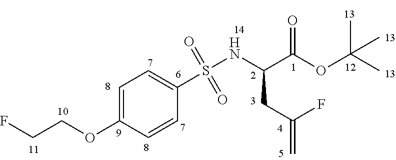

Prepared from tert-butyl (R)-2-amino-4-fluoropent-4-enecarboxylate (1000 mg, 5.28 mmol) and p-(2-fluorethoxy)phenylsulfonyl chloride (1225 mg, 5.28 mmol). Yield: 1.32 g (64%). M.p. 102° C. (EtOAc/Cy). ¹H NMR (300 MHz, CDCl₃): δ=7.79 (dm, ³J_{H,H}=9.0 Hz, 2H, 8-CH), 6.99 (dm, ³J_{H,H}=9.0 Hz, 2H, 7-CH), 5.37 (d, ³J_{H,H}=8.9 Hz, 1H, 14-NH), 4.78 (dm, ²J_{H,F}=47.3 Hz, 2H, 11-CH₂), 4.64 (dd, ³J_{H,F}=17.0 Hz, ²J_{H,H}=3.1 Hz, 1H, 5-CH_{cis}), 4.35 (dd, ³J_{H,F}=49.3 Hz, ²J_{H,H}=3.1 Hz, 1H, 5-CHₜᵣₐₙₛ), 4.26 (dm, ³J_{H,F}=27.7 Hz, 2H, 10-CH₂), 3.98 (dt, ³J_{H,H}=9.0 Hz, ³J_{H,H}=5.9 Hz, 1H, 2-CH), 2.65 (d, ³J_{H,H}=5.9 Hz, 1H, 3-CH_{A}), 2.59 (dd, ³J_{H,H}=5.7 Hz, ³J_{H,H}=4.2 Hz, 1H, 3-CH_{B}), 1.30 (s, 9H, 13-CH₃). ¹³C NMR (75 MHz, CDCl₃): δ=169.2 (s, C-1), 161.8 (s, C-9), 160.9 (ds, ¹J_{H,F}=257.1 Hz, C-4), 131.9 (s, C-6), 129.5 (d, C-8), 114.7 (d, C-7), 94.1 (dt, ²J_{H,F}=18.8 Hz, C-5), 83.2 (s, C-12), 81.5 (dt, ¹J_{H,F}=171.5 Hz, C-11), 67.4 (dt, ²J_{H,F}=20.4 Hz, C-10), 53.2 (d, C-2), 36.4 (dt, ²J_{H,F}=27.4 Hz, C-3), 27.6 (q, C-13). ¹⁹F NMR (282 MHz, CDCl₃): δ =−96.1 (ddt, ³J_{H,F}=49.3 Hz, ³J_{H,F}=20.4 Hz, ³J_{H,F}=17.2 Hz, 4-CF), −224.3 (tt, ²J_{H,F}=47.3 Hz, ³J_{H,F}=27.8 Hz, 11-CH₂F). Elemental analysis: C₁₇H₂₃F₂NO₅S (M=391.43 g/mol), alcld. C, 52.16; H, 5.92; N, 3.58. found C, 52.64; H, 6.24; N, 3.55%. Exact mass (ESI⁺): C₁₇H₂₃F₂NO₅S+Na⁺: cald. 414.1163. found 414.1160. (C₁₇H₂₃F₂NO₅S)₂+Na⁺: cald. 805.2428. found 805.2423. Optical rotation: [α]₅₈₉²⁰=−4.0, [α]₅₇₈²⁰=−4.4, [α]₅₄₆²⁰=−5.3, [α]₄₃₆²⁰=−13.0, [α]₃₆₅²⁰=−31.1 (c=1.023, CHCl₃)

### Example 24b

### Compound MAB 240

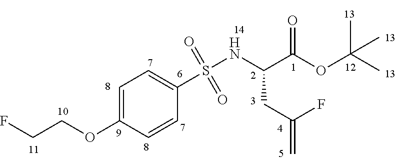

Prepared from tert-butyl (S)-2-amino-4-fluoropent-4-enecarboxylate (892 mg, 4.7 mmol) with p-(2-fluoroethoxy)-phenylsulfonylchloride (1130 mg, 4.7 mmol). Yield: 1.18 g (64%). M.p. 102° C. (EtOAc/Cy). ¹H NMR (300 MHz, CDCl₃): δ=7.79 (dm, ³J_{H,H}=9.0 Hz, 2H, 8-CH), 6.99 (dm, ³J_{H,H}=9.0 Hz, 2H, 7-CH), 5.37 (d, ³J_{H,H}=9.0 Hz, 1H, 14-NH), 4.77 (dm, ²J_{H,F}=47.5 Hz, 2H, 11-CH₂), 4.64 (dd, ³J_{H,F}=17.1 Hz, ²J_{H,H}=3.1 Hz, 1H, 5-CH_{cis}), 4.35 (dd, ³J_{H,F}=49.3 Hz, ²J_{H,H}=3.1 Hz, 1H, 5-CHₜᵣₐₙₛ), 4.26 (dm, ³J_{H,F}=27.7 Hz, 2H, 10-CH₂), 3.98 (dt, ³J_{H,H}=8.8 Hz, ³J_{H,H}=5.8 Hz, 1H, 2-CH), 2.65 (d, ³J_{H,H}=5.9 Hz, 1H, 3-CH_{A}), 2.59 (dd, ³J_{H,H}=5.7 Hz, ³J_{H,H}=4.2 Hz, 1H, 3-CH_{B}), 1.30 (s, 9H, 13-CH₃). ¹³C NMR (75 MHz, CDCl₃): δ=169.2 (s, C-1), 161.8 (s, C-9), 160.9 (ds, ¹J_{H,F}=256.9 Hz, C-4), 131.9 (s, C-6), 129.5 (d, C-8), 114.7 (d, C-7), 94.1 (dt, ²J_{H,F}=18.8 Hz, C-5), 83.2 (s, C-12), 81.5 (dt, ¹J_{H,F}=171.5 Hz, C-11), 67.4 (dt, ²J_{H,F}=20.4 Hz, C-10), 53.2 (d, ³J_{C,F}=1.0 Hz, C-2), 36.4 (dt, ²J_{C,F}=27.6 Hz, C-3), 27.6 (q, C-13). ¹⁹F NMR (282 MHz, CDCl₃): δ =−96.1 (ddt, ³J_{H,F}=49.3 Hz, ³J_{H,F}=20.4 Hz, ³J_{H,F}=17.2 Hz, 4-CF), −224.3 (tt, ²J_{H,F}=47.3 Hz, ³J_{H,F}=27.7 Hz, 11-CH₂F). Elemental analysis: C₁₇H₂₃F₂NO₅S (M=391.43 g/mol). Calcd. C, 52.16; H, 5.92; N, 3.58. found C, 52.19; H, 6.09; N, 3.54%. Optical rotation: [α]₅₈₉²⁰=−4.0, [α]₅₇₈²⁰=−4.4, [α]₅₄₆²⁰=−5.3, [α]₄₃₆²⁰=−13.0, [α]₃₆₅²⁰=−31.1 (c=1.005, CHCl₃).

### Example 25

### Example 25a

### Compound MAB 248

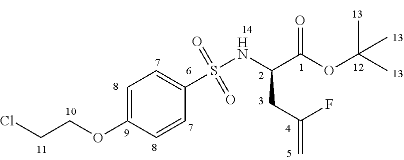

Prepared from tert-butyl (R)-2-amino-4-fluoropent-4-enoate (1000 mg, 5.28 mmol) and p-(2-chlorethoxy)-phenylsulfonylchloride (1350 mg, 5.28 mmol). Yield: 1.29 g (61%). M.p. 98-99° C. (EtOAc/Cy). ¹H NMR (300 MHz, CDCl₃): δ=7.80 (dm, ³J_{H,H}=9.0 Hz, 2H, 8-CH), 6.98 (dm, ³J_{H,H}=9.0 Hz, 2H, 7-CH), 5.29 (d, ³J_{H,H}=9.0 Hz, 1H, 14-NH), 4.65 (dd, ³J_{H,F}=17.0 Hz, ²J_{H,H}=3.1 Hz, 1H, 5-CH_{cis}), 4.35 (q, ³J_{H,F}=49.5 Hz, ²J_{H,H}=2.9 Hz, 2H, 5-CHₜᵣₐₙₛ), 4.27 (t, ³J_{H,H}=5.7 Hz, 2H, 10-CH₂), 3.98 (dt, ³J_{H,H}=8.9 Hz, ³J_{H,H}=5.7 Hz, 1H, 2-CH), 3.84 (t, ³J_{H,H}=5.7 Hz, 2H, 11-CH₂Cl), 2.65 (dd, ³J_{H,H}=5.8 Hz, ²J_{H,H}=0.9 Hz, 1H, 3-CH_{A}), 2.59 (t, ³J_{H,H}=5.3 Hz, 1H, 3-CH_{B}), 1.30 (s, 9H, 13-CH₃). ¹³C NMR (75 MHz, CDCl₃): δ=169.2 (s, C-1), 161.6 (s, C-9), 160.9 (ds, ¹J_{H,F}=257.1 Hz, C-4), 132.0 (s, C-6), 129.5 (d, C-8), 114.7 (d, C-7), 94.2 (dt, ²J_{H,F}=18.9 Hz, C-5), 83.2 (s, C-12), 68.2 (t, C-10), 53.2 (dd, ³J_{H,F}=0.9 Hz, C-2), 41.5 (t, C-11), 36.5 (dt, ²J_{H,F}=27.2 Hz, C-3), 27.7 (q, C-13). ¹⁹F NMR (282 MHz, CDCl₃): δ =−96.1 (ddt, ³J_{H,F}=49.2 Hz, ³J_{H,F}=20.6 Hz, ³J_{H,F}=17.1 Hz, 4-CF). Elemental analysis: C₁₇H₂₃ClFNO₅S (M=407.88 g/mol); calcd. C, 50.06; H, 5.68; N, 3.43. found C, 50.02, H, 5.51N, 3.36%. Exact mass (ESI⁺): C₁₇H₂₃ClFNO₅S+Na⁺; calcd. 430.0867. found 430.0851; (C₁₇H₂₃ClFNO₅S)₂+Na⁺; calcd. 837.1837. found 837.1824. Optical rotation: [α]₅₈₉²⁰=+6.3, [α]₅₇₈²⁰=+6.9, [α]₅₄₆²⁰=+8.1, [α]₄₃₆²⁰=+18.5, [α]₃₆₅²⁰=+42.5 (c=0.991, CHCl₃).

### Example 25b

### Compound MAB 249

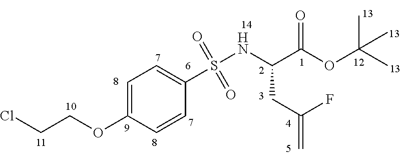

Prepared from tert-butyl (S)-2-amino-4-fluoropent-4-enoate (1000 mg, 5.28 mmol) and p-(2-chlorethoxy)phenylsulfonylchloride (1350 mg 5.28 mmol). Yield: 1.58 g (73%). ¹H NMR (300 MHz, CDCl₃): δ=7.80 (dm, ³J_{H,H}=9.0 Hz, 2H, 8-CH), 6.98 (dm, ³J_{H,H}=9.0 Hz, 2H, 7-CH), 5.32 (d, ³J_{H,H}=8.9 Hz, 1H, 14-NH), 4.64 (dd, ³J_{H,F}=17.1 Hz, ²J_{H,H}=3.0 Hz, 1H, 5-CH_{cis}), 4.35 (q, ³J_{H,F}=49.4 Hz, ²J_{H,H}=3.0 Hz, 2H, 5-CHₜᵣₐₙₛ), 4.27 (t, ³J_{H,H}=5.7 Hz, 2H, 10-CH₂), 3.98 (dt, ³J_{H,H}=9.0 Hz, ³J_{H,H}=5.8 Hz, 1H, 2-CH), 3.84 (t, ³J_{H,H}=5.7 Hz, 2H, 11-CH₂Cl), 2.65 (dd, ³J_{H,H}=5.9 Hz, 1H, 3-CH_{A}), 2.59 (m, ³J_{H,H}=5.4 Hz, 1H, 3-CH_{B}), 1.30 (s, 9H, 13-CH₃). ¹³C NMR (75 MHz, CDCl₃): δ=169.2 (s, C-1), 161.6 (s, C-9), 160.9 (ds, ¹J_{H,F}=257.1 Hz, C-4), 132.0 (s, C-6), 129.5 (d, C-8), 114.7 (d, C-7), 94.2 (dt, ²J_{H,F}=18.8 Hz, C-5), 83.2 (s, C-12), 68.2 (t, C-10), 53.2 (dd, ³J_{H,F}=0.9 Hz, C-2), 41.5 (t, C-11), 36.5 (dt, ²J_{H,F}=27.6 Hz, C-3), 27.6 (q, C-13). ¹⁹F NMR (282 MHz, CDCl₃): δ =−96.1 (ddt, ³J_{H,F}=49.4 Hz, ³J_{H,F}=20.5 Hz, ³J_{H,F}=17.1 Hz, 1H). Elemental analysis: C₁₇H₂₃ClFNO₅S (M=407.88 g/mol), calcd. C, 50.06; H, 5.68; N, 3.43. found C, 49.90; H, 5.53; N, 3.38%. Exact mass (ESI⁺): C₁₇H₂₃ClFNO₅S+Na⁺, calcd. 430.0867. found 430.0856; (C₁₇H₂₃ClFNO₅S)₂+Na⁺, calcd. 837.1837. found 837.1835. Optical rotation: [α]₅₈₉²⁰=−2.6, [α]₅₇₈²⁰=−2.9, [α]₅₄₆²⁰=−3.5, [α]₄₃₆²⁰=−8.5, [α]₃₆₅²⁰=−19.8 (c=1.021, CHCl₃).

General Procedure for Substitution with Benzyl Bromide

The N-substituted tert-butyl 2-amino-4-fluoropent-4-enoate dissolved in dimethylformamide is treated under stirring with 10 equivalents of potassium carbonate. After 20 min 1 equivalent of benzyl bromide is added and the mixture is stirred at r.t. for 40 h. Then water is added and the mixture is extracted with ethyl acetate (4 times). The combined organic extracts are washed with water (4 times), saturated sodium chloride solution and dried with magnesium sulfate. After removal of the solvent the crude product is purified chromatographically an (silica gel, Cy/EtOAc, 4:1). The products are obtained as colorless or yellowish viscos oils.

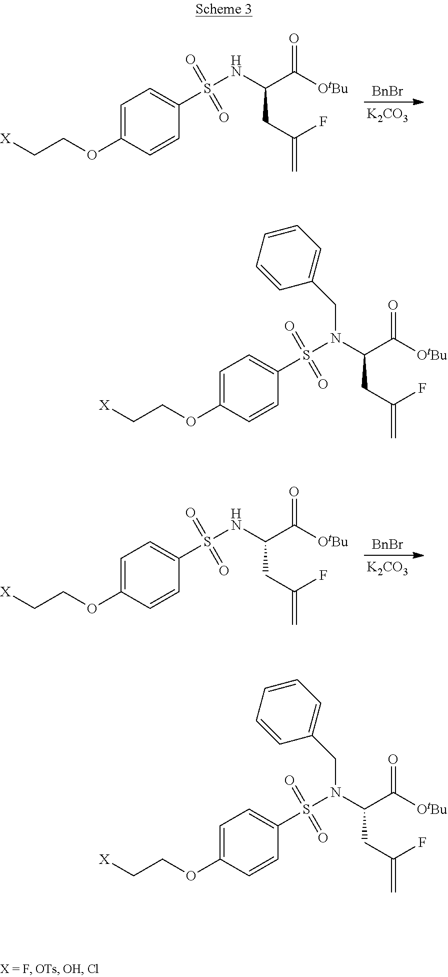

### Example 26

### Example 26a

### Compound MAB 242

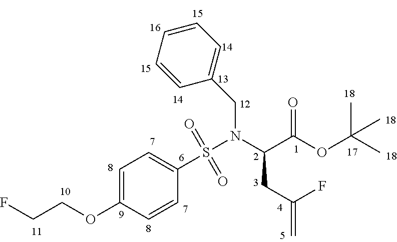

Prepared from tert-butyl N-[p-(2-fluoroethoxy)phenylsulfonyl]aminopent-4-enoate (783 mg, 2 mmol), benzyl bromide (342 mg, 2 mmol) and potassium carbonate 2.8 g (20 mmol). Yield: 600 mg (63%). ¹H NMR (300 MHz, CDCl₃): δ=7.82 (dm, ³J_{H,H}=9.0 Hz, 2H, 8-CH), 7.29 (m, 5H, 14-CH, 15-CH, 16-CH), 6.98 (dm, ³J_{H,H}=9.0 Hz, 2H, 7-CH), 4.80 (dm, ²J_{H,F}=47.1 Hz, 2H, 11-CH₂), 4.66 (d, ²J_{H,H}=16.3 Hz, 1H, 12-CH_{A}), 4.56 (t, ³J_{H,H}=7.3 Hz, 1H, 2-CH), 4.53 (dd, ³J_{H,F}=17.1 Hz, ²J_{H,H}=2.9 Hz, 1H, 5-CH_{cis}), 4.29 (d, ²J_{H,H}=15.9 Hz, 2H, 12-CH_{B}), 4.27 (dm, ³J_{H,F}=27.8 Hz, 2H, 10-CH₂), 4.11 (dd, ³J_{H,F}=49.8 Hz, ²J_{H,H}=3.1 Hz, 1H, 5-CHₜᵣₐₙₛ), 2.70 (dt, ³J_{H,F}=15.4 Hz, ³J_{H,H}=6.9 Hz, 1H, 3-CH_{A}), 2.46 (ddd, ³J_{H,F}=20.0 Hz, ²J_{H,H}=15.1 Hz, ³J_{H,H}=7.6 Hz, 1H, 3-CH_{B}), 1.36 (s, 9H, 18-CH₃). ¹³C NMR (75 MHz, CDCl₃): δ=168.5 (s, C-1), 161.9 (ds, ¹J_{H,F}=256.5 Hz, C-4), 161.7 (s, C-9), 136.7 (s, C-13), 132.5 (s, C-6), 129.9 (d, C-8), 128.5 (d, C-14 o. C-15), 128.4 (d, C-14 o. C-15), 127.7 (d, C-16), 114.6 (d, C-7), 93.1 (dt, ²J_{H,F}=19.1 Hz, C-5), 82.6 (s, C-17), 81.5 (dt, ¹J_{H,F}=171.7 Hz, C-11), 67.4 (dt, ²J_{H,F}=20.5 Hz, C-10), 57.6 (d, C-2), 50.0 (t, C-12), 34.1 (dt, ²J_{H,F}=27.9 Hz, C-3), 27.8 (q, C-18). ¹⁹F NMR (282 MHz, CDCl₃): δ =−97.7 (m, ³J_{H,F}=49.8 Hz, ³J_{H,F}=20.0 Hz, ³J_{H,F}=17.0 Hz, 4-CF), −224.3 (tt, ²J_{H,F}=47.3 Hz, ³J_{H,F}=27.6 Hz, 11-CH₂F). Exact mass (ESI⁺): C₂₄H₂₉F₂NO₅S+Na⁺, calcd. 504.1632. found 504.1615; (C₂₄H₂₉F₂NO₅S)₂+Na⁺, calcd. 985.3367. found 985.3348. Optical rotation: [α]₅₈₉²⁰=−22.8, [α]₅₇₈²⁰=−23.8, [α]₅₄₆²⁰=−27.2, [α]₄₃₆²⁰=−49.6, [α]₃₆₅²⁰=−80.6 (c=1.018 CHCl₃).

### Example 26b

### Compound MAB 243

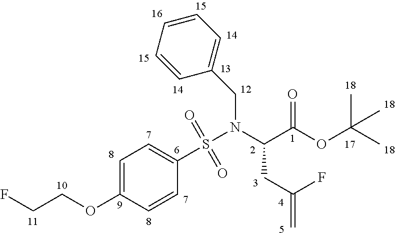

Prepared from tert-butyl N-[p-(-2-fluoroethoxy)phenylsulfonyl]aminopent-4-enoate (830 mg, 2.12 mmol), benzyl bromide (363 mg, 2.12 mmol) and 2.93 g (21.2 mmol) potassium carbonate. Yield: 800 mg (1.7 mmol, 78%). ¹H NMR (300 MHz, CDCl₃): δ=7.82 (dm, ³J_{H,H}=9.0 Hz, 2H, 8-CH), 7.29 (m, 5H, 14-CH, 15-CH, 16-CH), 6.98 (dm, ³J_{H,H}=8.9 Hz, 2H, 7-CH), 4.80 (dm, ²J_{H,F}=47.3 Hz, 2H, 11-CH₂), 4.65 (d, ²J_{H,H}=16.0 Hz, 1H, 12-CH_{A}), 4.56 (t, ³J_{H,H}=7.3 Hz, 1H, 2-CH), 4.53 (dd, ³J_{H,F}=17.1 Hz, ²J_{H,H}=3.0 Hz, 1H, 5-CH_{cis}), 4.29 (d, ²J_{H,H}=15.7 Hz, 2H, 12-CH_{B}), 4.25 (dm, ³J_{H,F}=27.8 Hz, 2H, 10-CH₂), 4.11 (dd, ³J_{H,F}=, 49.9 Hz, ²J_{H,H}=3.0 Hz, 1H, 5-CHₜᵣₐₙₛ), 2.70 (dt, ³J_{H,F}=15.3 Hz, ³J_{H,H}=6.9 Hz, 1H, 3-CH_{A}), 2.46 (ddd, ³J_{H,F}=20.0 Hz, ²J_{H,H}=15.1 Hz, ³J_{H,H}=7.6 Hz, 1H, 3-CH_{B}), 1.36 (s, 9H, 18-CH₃). ¹³C NMR (75 MHz, CDCl₃): δ=168.5 (s, C-1), 161.9 (ds, ¹J_{H,F}=256.5 Hz, C-4), 161.7 (s, C-9), 136.7 (s, C-13), 132.5 (s, C-6), 129.9 (d, C-8), 128.5 (d, C-14 o. C-15), 128.4 (d, C-14 o. C-15), 127.7 (d, C-16), 114.6 (d, C-7), 93.1 (dt, ²J_{H,F}=19.2 Hz, C-5), 82.6 (s, C-17), 81.5 (dt, ¹J_{C,F}=171.6 Hz, C-11), 67.4 (dt, ²J_{C,F}=20.2 Hz, C-10), 57.6 (d, C-2), 50.0 (t, C-12), 34.1 (dt, ²J_{C,F}=27.9 Hz, C-3), 27.8 (q, C-18). ¹⁹F NMR (282 MHz, CD₃CN): δ =−97.7 (m, ³J_{H,F}=49.8 Hz, ³J_{H,F}=19.8 Hz, ³J_{H,F}=16.4.0 Hz, 4-CF), −224.3 (tt, ²J_{H,F}=47.3 Hz, ³J_{H,F}=27.6 Hz, 11-CH₂F). Exact mass (ESI⁺): C₂₄H₂₉F₂NO₅S+Na⁺, calcd. 504.1632. found 504.1613. (C₂₄H₂₉F₂NO₅S)₂+Na⁺, calcd. 985.3367. found 985.3345. Optical rotation: [α]₅₈₉²⁰=+19.9, [α]₅₇₈²⁰=+20.3, [α]₅₄₆²⁰=+23.2, [α]₄₃₆²⁰=+42.5, [α]₃₆₅²⁰=+74.0 (c=1.018, CHCl₃).

### Example 27

### Example 27a

### Compound MAB 250

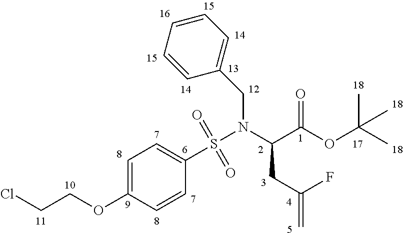

Prepared from tert-butyl N-[p-(2-chloroethoxy)phenylsulfonyl]-2-amino-4-fluoropent-4-enoate (816 mg 2.0 mmol), benzyl bromide (342 mg, 2.0 mmol) and potassium carbonate 2.8 g (20.0 mmol). Yield: 580 mg (58%). ¹H NMR (300 MHz, CDCl₃): δ=7.82 (dm, ³J_{H,H}=8.9 Hz, 2H, 8-CH), 7.29 (m, 5H, 14-CH, 15-CH, 16-CH), 6.96 (dm, ³J_{H,H}=9.0 Hz, 2H, 7-CH), 4.65 (d, ²J_{H,H}=15.8 Hz, 1H, 12-CH_{A}), 4.56 (t, ³J_{H,H}=7.3 Hz, 1H, 2-CH), 4.53 (dd, ³J_{H,F}=17.2 Hz, ²J_{H,H}=2.9 Hz, 1H, 5-C_{cis}), 4.29 (d, ²J_{H,H}=15.7 Hz, 1H, 12-CH_{B}), 4.27 (t, ³J_{H,H}=5.6 Hz, 1H, 10-CH₂), 4.11 (dd, ³J_{H,F}=49.9 Hz, ²J_{H,H}=3.0 Hz, 1H, 5-CHₜᵣₐₙₛ), 3.84 (t, ³J_{H,H}=5.7 Hz, 2H, 11-CH₂Cl), 2.70 (td, ²J_{H,H}=15.4 Hz, ³J_{H,H}=7.0 Hz, 1H, 3-CH_{A}), 2.46 (ddd, ³J_{H,F}=20.0 Hz, ²J_{H,H}=15.2 Hz, ³J_{H,H}=7.6 Hz, 1H, 3-CH_{B}), 1.36 (s, 9H, 18-CH₃). ¹³C NMR (75 MHz, CDCl₃): δ=168.5 (s, C-1), 161.9 (ds, ¹J_{H,F}=256.5 Hz, C-4), 161.5 (s, C-9), 136.7 (s, C-13), 132.6 (s, C-6), 129.9 (d, C-8), 128.5 (d, C-14 o. C-15), 128.4 (d, C-14 o. C-15), 127.7 (d, C-16), 114.6 (d, C-7), 93.1 (dt, ²J_{C,F}=18.9 Hz, C-5), 82.6 (s, C-17), 68.2 (t, C-10), 57.6 (d, C-2), 49.9 (t, C-12), 41.5 (t, C-11), 34.1 (dt, ²J_{H,F}=28.2 Hz, C-3), 27.8 (q, C-18). ¹⁹F NMR (282 MHz, CDCl₃): δ =−97.7 (ddt, ³J_{H,F}=49.9 Hz, ³J_{H,F}=19.9 Hz, ³J_{H,F}=16.4 Hz, 4-CF). Exact mass (ESI⁺): C₂₄H₂₉ClFNO₅S+Na⁺, calcd. 520.1337. found 520.1314; (C₂₄H₂₉ClFNO₅S)₂+Na⁺, calcd. 1017.2776. found 1017.2708. Optical rotation: [α]₅₈₉²⁰=−24.8, [α]₅₇₈²⁰=−26.2, [α]₅₄₆²⁰=−30.0, [α]₄₃₆²⁰=−54.4, [α]₃₆₅²⁰=−95.0 (c=1.038, CHCl₃).

### Example 27b

### Compound MAB 251

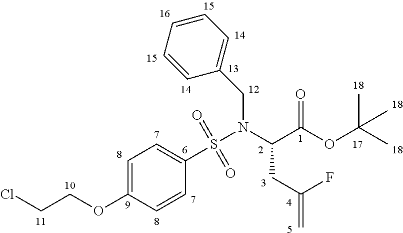

Prepared from tert-butyl N-[p-(2-chloroethoxy)phenylsulfonyl]-2-amino-4-fluoropent-4-enoate (1.50 g, 3.68 mmol), benzyl bromide (630 mg, 3.68 mmol) and potassium carbonate (5.0 g, 36.8 mmol). Yield: 1.28 g (70%). ¹H NMR (300 MHz, CDCl₃): δ=7.82 (dm, ³J_{H,H}=9.0 Hz, 2H, 8-CH), 7.28 (m, 5H, 14-CH, 15-CH, 16-CH), 6.96 (dm, ³J_{H,H}=9.0 Hz, 2H, 7-CH), 4.66 (d, J=15.8, 1H, 12-CH_{A}), 4.56 (m, 1H, 2-CH), 4.53 (dd, J=17.1 Hz, 3.0 Hz, 2H, 5-CH_{cis}), 4.29 (d, J=15.7 Hz, 1H, 12-CH_{B}), 4.28 (t, J=5.6 Hz, 2H, 10-CH₂), 4.11 (dd, J=49.9 Hz, 3.0 Hz, 1H, 5-CHₜᵣₐₙₛ), 3.83 (t, J=5.8 Hz, 2H, 11-CH₂Cl), 2.70 (td, J=15.4 Hz, 6.9 Hz, 1H, trans, 3-CH_{A}), 2.46 (ddd, J=20.1 Hz, 15.2 Hz, 7.6 Hz, 1H, 3-CH_{B}), 1.36 (s, 9H, 18-CH₃). ¹³C NMR (75 MHz, CDCl₃): δ=168.6 (s, C-1), 161.9 (ds, ¹J_{H,F}=256.3 Hz, C-4), 161.5 (s, C-9), 136.7 (s, C-13), 132.7 (s, C-6), 129.9 (d, C-8), 128.5 (d, C-14 o. C-15), 128.4 (d, C-14 o. C-15), 127.8 (d, C-16), 114.6 (d, C-7), 93.1 (dt, ²J_{H,F}=19.1 Hz, C-5), 82.6 (s, C-17), 68.2 (t, C-10), 57.6 (d, C-2), 50.0 (t, C-12), 41.5 (t, C-11), 34.1 (dt, ²J_{H,F}=27.9 Hz, C-3), 27.8 (q, C-18). ¹⁹F NMR (282 MHz, CDCl₃): δ =−97.7 (m, ³J_{H,F}=49.9 Hz, ³J_{H,F}=19.9 Hz, ³J_{H,F}=16.7 Hz, 4-CF). Exact mass (ESI⁺): C₂₄H₂₉ClFNO₅S+Na⁺, calcd. 520.1337. found 520.1326; (C₂₄H₂₉ClFNO₅S)₂+Na⁺, calcd. 1017.2776. found 1017.2748. Optical rotation: [α]₅₈₉²⁰=+21.1, [α]₅₇₈²⁰=+22.2, [α]₅₄₆²⁰=+25.1, [α]₄₃₆²⁰=+45.5, [α]₃₆₅²⁰=n.d. (c=1.005, CHCl₃).

Hydrolysis of the Tert-Butylesters

In a dried YOUNG-tube the amino acid tert-butylester is dissolved in dry dichloromethane (20 mL/mmol) under argon and treated with trifluoroacetic acid (20 mL/mmol). The YOUNG-tube is flushed with argon and sealed. The mixture is stirred at r.t. for 3-4 h. Subsequently the reaction mixture is evaporated to dryness in vacuum. The residue is dissolved in chloroform (100 mL/mmol) and washed with an aqueous solution of citric acid and sodium bicarbonate (25 mL/mmol, pH≈4). The aqueous phase is extracted with chloroform (4×30 mL/mmol) and the combined organic phases are dried with magnesium sulfate. After evaporation of the solvent the crude products are obtained as colorless, viscos oils, which in high vacuum solidified. These products were used for the next step without purification.

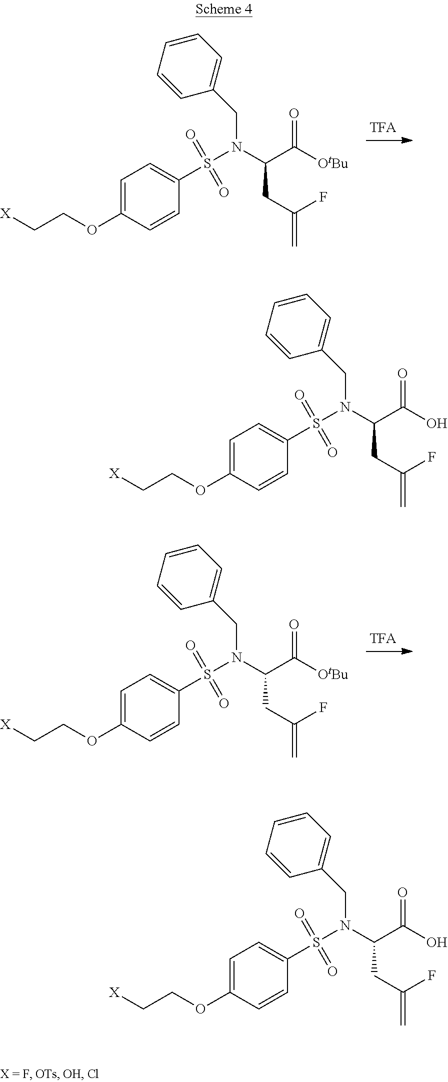

### Example 28

### Example 28a

### Compound MAB 252

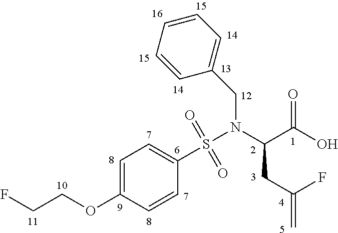

Prepared from tert-butyl N-benzyl-N-[p-(2-fluoroethoxy)phenylsulfonyl]-2-amino-4-fluoropent-4-enoate (300 mg 0.7 mmol). ¹H NMR (300 MHz, CD₃CN): δ=7.78 (dm, ³J_{H,H}=9.0 Hz, 2H, 8-CH), 7.30 (m, 5H, 14-CH, 15-CH, 16-CH), 7.04 (dm, ³J_{H,H}=9.0 Hz, 2H, 7-CH), 4.76 (dm, ²J_{H,F}=47.7 Hz, 2H, 11-CH₂), 4.65 (dd, ³J_{H,H}=8.5 Hz, ³J_{H,H}=6.0 Hz, 1H, 2-CH), 4.57 (d, ²J_{H,H}=16.1 Hz, 1H, 12-CH_{A}), 4.52 (dd, ³J_{H,F}=17.8 Hz, ²J_{H,H}=3.0 Hz, 1H, 5-CH_{cis}), 4.33 (d, ²J_{H,H}=16.1 Hz, 1H, 12-CH_{B}), 4.29 (dm, ³J_{H,F}=29.5 Hz, 2H, 10-CH₂), 4.14, (dd, ³J_{H,F}=51.5 Hz, ³J_{H,H}=3.5 Hz, 1H, 5-CHₜᵣₐₙₛ 2.76 (ddd, ³J_{H,F}=14.8 Hz, ²J_{H,H}=13.7 Hz, ³J_{H,H}=6.0 Hz, 1H, 3-CH_{A}), 2.48 (ddd, ³J_{H,F}=22.3 Hz, ²J_{H,H}=15.4 Hz, ³J_{H,H}=8.5 Hz, 1H, 3-CH_{B}). ¹³C NMR (75 MHz, CD₃CN): δ=171.2 (s, C-1), 163.2 (ds, ¹J_{H,F}=254.7 Hz, C-4), 163.1 (s, C-9), 138.2 (s, C-13), 133.0 (s, C-6), 131.0 (d, C-8), 129.6 (d, C-14 o. C-15), 129.3 (d, C-14 o. C-15), 128.7 (d, C-16), 115.7 (d, C-7), 93.8 (dt, ²J_{H,F}=18.9 Hz, C-5), 83.1 (dt, ¹J_{H,F}=167.3 Hz, C-11), 68.8 (dt, ²J_{C,F}=19.4 Hz, C-10), 57.9 (d, C-2), 50.8 (t, C-12), 34.1 (dt, ²J_{H,F}=28.2 Hz, C-3). ¹⁹F NMR (282 MHz, CD₃CN): δ =−97.7 (dddd, ³J_{H,F}=51.0 Hz, ³J_{H,F}=22.1 Hz, ³J_{H,F}=17.7 Hz, ³J_{H,F}=13.4 Hz, 4-CF), −224.0 (tt, ²J_{H,F}=47.7 Hz, ³J_{H,F}=29.5 Hz, 11-CH₂F). Exact mass (ESI⁺): C₂₀H₃₁F₂NO₅S+Na⁺, calcd. 448.1006. found 448.1004, (C₂₀H₃₁F₂NO₅S)₂+Na⁺, calcd. 873.2115. found 873.2116. Exact mass (ESI⁻): C₂₀H₃₁F₂NO₅S—H⁺, calcd. 424.1036. found 424.1057, (C₂₀H₃₁F₂NO₅S)₂—H⁺, calcd. 849.2144. found 849.2152.

### Example 28b

### Compound MAB 253

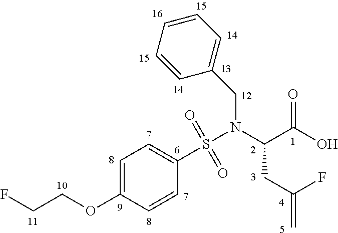

Prepared from tert-butyl N-benzyl-N-[p-(2-fluoroethoxy)phenylsulfonyl]-2-amino-4-fluoropent-4-enoate (400 mg, 0.94 mmol). The product is contaminated with some starting material. ¹H NMR (300 MHz, CD₃CN): δ=7.78 (dm, ³J_{H,H}=9.1, 2H, 8-CH), 7.29 (m, 5H, 14-CH, 15-CH, 16-CH), 7.04 (dm, ³J_{H,H}=9.0, 2H, 7-CH), 4.75 (dm, ²J_{H,F}=47.7 Hz, 2H, 11-CH₂), 4.65 (dd, ³J_{H,H}=8.5, ³J_{H,H}=6.0, 1H, 2-CH), 4.57 (d, ²J_{H,H}=16.1, 1H, 12-CH_{A}), 4.52 (dd, ³J_{H,F}=17.6, ²J_{H,H}=3.0, 1H, 5-CH_{cis}), 4.32 (d, ²J_{H,H}=16.1, 1H, 12-CH_{B}), 4.30 (dm, J_{H,F}=29.4 Hz, 2H, 10-CH₂), 4.14 (dd, ³J_{H,F}=51.8, ²J_{H,H}=3.4, 1H, 5-CHₜᵣₐₙₛ, 2.76 (ddd, ³J_{H,F}=14.7, ²J_{H,H}=13.7, ³J_{H,H}=6.0, 1H, 3-CH_{A}), 2.47 (ddd, ³J_{H,F}=22.2, ²J_{H,H}=15.4, ³J_{H,H}=8.5, 1H, 3-CH_{B}). ¹³C NMR (75 MHz, CD₃CN): δ=171.2 (s, C-1), 163.2 (ds, ¹J_{H,F}=254.7 Hz, C-4), 163.1 (s, C-9), 138.2 (s, C-13), 133.0 (s, C-6), 131.0 (d, C-8), 129.6 (d, C-14 o. C-15), 129.3 (d, C-14 o. C-15), 128.7 (d, C-16), 115.7 (d, C-7), 93.8 (dt, ²J_{H,F}=18.9 Hz, C-5), 83.1 (dt, ¹J_{H,F}=167.5 Hz, C-11), 68.8 (dt, ²J_{C,F}=19.2 Hz, C-10), 57.9 (d, C-2), 50.8 (t, C-12), 34.2 (dt, ²J_{H,F}=28.0 Hz, C-3). ¹⁹F NMR (282 MHz, CD₃CN): δ =−97.7 (dddd, ³J_{H,F}=50.9 Hz, ³J_{H,F}=22.1 Hz, ³J_{H,F}=17.7 Hz, ³J_{H,F}=13.3 Hz, 4-CF), −224.0 (tt, ²J_{H,F}=47.7 Hz, ³J_{H,F}=29.5 Hz, 11-CH₂F). Exact mass (ESI⁺): C₂₀H₃₁F₂NO₅S+Na⁺, calcd. 448.1006. found 448.1009; Exact mass (ESI⁻): C₂₀H₃₁F₂NO₅S—H⁺, calcd. 424.1036. found 424.1057, (C₂H₃₁F₂NO₅S)₂—H⁺, calcd. 849.2144. found 849.2157.

### Example 29

### Example 29a

### Compound MAB 256

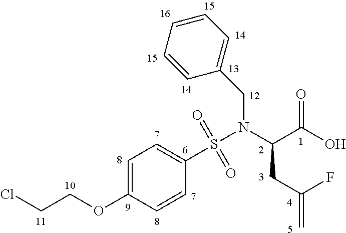

Prepared from tert-butyl N-benzyl-N-[p-(2-chloroethoxy)phenylsulfonyl]-2-amino-4-fluoropent-4-enoate (300 mg, 0.68 mmol). ¹H NMR (300 MHz, CD₃CN): δ=7.78 (d, ³J_{H,H}=9.1 Hz, 2H, 8-CH), 7.29 (m, 5H, 14-CH, 15-CH, 16-CH), 7.03 (d, ³J_{H,H}=9.1 Hz, 1H, 7-CH), 4.65 (dd, ³J_{H,H}=8.5 Hz, ³J_{H,H}=6.0 Hz, 1H, 2-CH), 4.57 (d, ²J_{H,H}=16.1 Hz, 1H, 12-CH_{A}), 4.52 (dd, ³J_{H,F}=17.7 Hz, ²J_{H,H}=3.2 Hz, 1H, 5-CH_{cis}), 4.33 (m, 2H, 10-CH₂), 4.32 (d, ²J_{H,H}=16.1 Hz, 1H, 12-CH_{B}), 4.14 (dd, ³J_{H,F}=50.9 Hz, ²J_{H,H}=3.1 Hz, 1H, 5-CHₜᵣₐₙₛ), 3.90 (m, 2H, 11-CH₂Cl), 2.76 (ddd, ³J_{H,F}=16.1 Hz, ²J_{H,H}=14.2 Hz, ³J_{H,H}=5.9 Hz, 2H), 2.47 (ddd, ³J_{H,F}=22.3 Hz, ²J_{H,H}=15.5 Hz, ³J_{H,H}=8.6 Hz, 1H). ¹³C NMR (75 MHz, CD₃CN): δ=171.2 (s, C-1), 163.2 (d, ¹J_{H,F}=254.5 Hz, C-4), 162.9 (s, C-9), 138.2 (s, C-13), 133.1 (s, C-6), 131.0 (s, C-8), 129.6 (s, C-14 or C-15), 129.3 (s, C-14 or C-15), 128.7 (s, C-16), 115.7 (s, C-7), 93.8 (d, ²J_{H,F}=18.9 Hz, C-5), 69.6 (s, C-10), 57.9 (s, C-2), 50.8 (s, C-12), 43.5 (s, C-11), 34.1 (d, ²J_{C,F}=28.4 Hz, C-3). ¹⁹F NMR (282 MHz, CD₃CN): δ =−97.7 (dddd, ³J_{H,F}=51.0 Hz, ³J_{H,F}=22.2 Hz, ³J_{H,F}=17.7 Hz, ³J_{H,F}=13.3 Hz, 4-CF).

### Example 29b

### Compound MAB 257

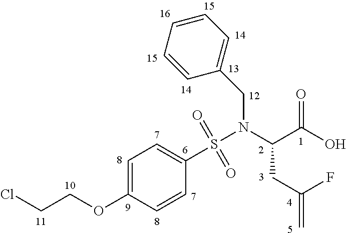

Prepared from tert-butyl N-benzyl-N-[p-(2-chloroethoxy)phenylsulfonyl]-2-amino-4-fluoropent-4-enoate (430 mg, 0.97 mmol). The crude product was directly used for the next step.

Synthesis of the tert-butyl hydroxamates

The N,N-disubstituted α-amino acid (1 equivalent) is suspended in dichloromethane (15 mL/mmol). 1-Hydroxy-benzotriazol (HOBT, 1 equivalent) is added and the mixture is stirred for a short period of time. Then N-methylmorpholin (NMM, 5 equivalents) is added under stirring while the carboxylic acid dissolves. Subsequently, O-tert-butylhydroxylamine (3 aquivalents) is added and the solution is stirred for 5 min. After addition of 1-(3-dimethylaminopropyl-3-ethylcarbodiimid-hydrochloride (EDC) (1.3 equivalents) the mixture is stirred at r.t. over night. The solution is washed with water (20 mL), and the aqueous phase is extracted with dichloromethane (3×20 mL). The combined organic phase is washed with brine (1×20 mL) and dried with magnesium sulfate. The solvent is evaporated and the crude product is purified by column chromatography (silica gel, Cy/EtOAc, 1:3). The silica gel has to be inactivated using the eluent mixture with 2% of triethylamine. The products are obtained as highly viscose liquids, which on drying in high vacuum give amorphous solids.

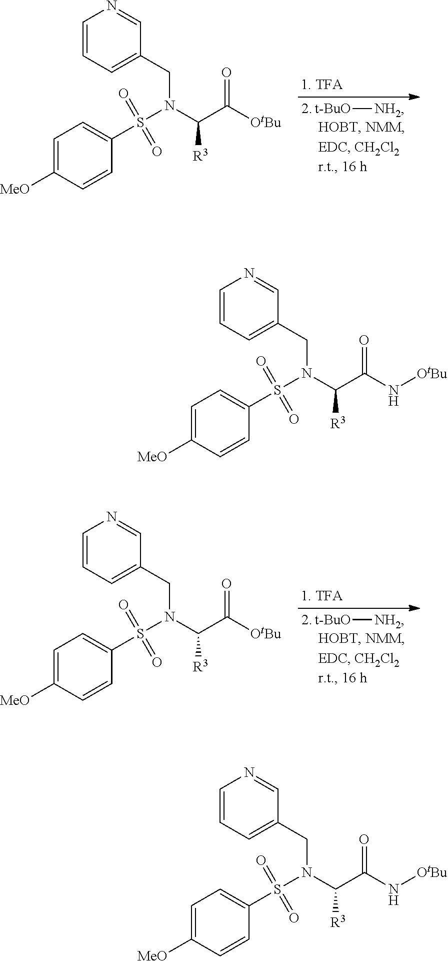

wherein R³ is CH₂CH₃, CH₂CH₂F, CH₂CH═CH₂, CH₂CF═CH₂.

### Example 30

### Example 30a

### Compound MAB 254

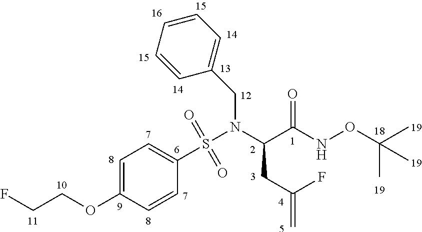

Prepared from N-benzyl-N-[p-(2-fluoroethoxy)phenylsulfonyl]-2-amino-4-fluoropent-4-ene carboxylic acid (191 mg, 0.45 mmol), HOPT (62 mg, 0.45 mmol), NMM (240 mg, 2.25 mmol), O-tert-butylhydroxylamine (175 mg, 1.35 mmol) and EDC (115 mg, 0.58 mmol). Yield: 147 mg (66%). ¹H NMR (300 MHz, CDCl₃): δ=8.54 (s, 1H, 17-NH), 7.69 (dm, ³J_{H,H}=9.0 Hz, 2H, 8-CH), 7.29 (m, 5H, 14-CH, 15-CH, 16-CH), 6.95 (dm, ³J_{H,H}=9.0 Hz, 2H, 7-CH), 4.77 (dm, ²J_{H,F}=47.3 Hz, 2H, 11-CH₂), 4.64 (d, ²J_{H,H}=15.7 Hz, 1H, 12-CH_{A}), 4.54 (t, ³J_{H,H}=7.2 Hz, 1H, 2-CH), 4.42 (dd, ³J_{H,F}=17.1 Hz, ²J_{H,H}=2.7 Hz, 2H, 5-CH_{cis}), 4.42 (d, overlapped with dd at 4.42), ²J_{H,H}=15.8 Hz, 1H, 12-CH_{B}) 4.25 (m, ³J_{H,F}=27.8 Hz, 2H, 10-CH₂), 4.05 (dd, ³J_{H,F}=50.0 Hz, ²J_{H,H}=3.0 Hz, 1H, 5-CHₜᵣₐₙₛ), 2.81 (ddd, ³J_{H,F}=18.3 Hz, trans, 9 ²J_{H,H}=14.9 Hz, ³J_{H,H}=7.6 Hz, 1H, 3-CH_{A}), 2.32 (ddd, ³J_{H,F}=19.1 Hz, ²J_{H,H}=14.9 Hz, ³J_{H,H}=7.0 Hz, 1H, 3-CH_{B}), 1.22 (s, 9H, 19-CH₃). ¹³C NMR (75 MHz, CDCl₃): δ=167.0 (s, C-1), 161.9 (s, C-9), 161.3 (ds, ¹J_{H,F}=256.0 Hz, C-4), 136.5 (s, C-13), 131.7 (s, C-6), 129.6 (d, C-8), 128.7 (d, C-14 o. C-15), 128.5 (d, C-14 o. C-15), 127.8 (d, C-16), 114.7 (d, C-7), 93.7 (dt, ²J_{H,F}=19.1 Hz, C-5), 82.3 (s, C-18), 81.4 (dt, ¹J_{H,F}=171.5 Hz, C-11), 67.3 (dt, J_{C,F}=20.4 Hz, C-10), 55.0 (d, C-2), 48.6 (t, C-12), 32.4 (dt, ²J_{H,F}=27.4 Hz, C-3), 26.1 (q, C-19). ¹⁹F NMR (282 MHz, CDCl₃): δ =−97.92 (m, ³J_{H,F}=50.1 Hz, ³J_{H,F}=18.4 Hz, 4-CF), −223.79 (tt, ³J_{H,F}=47.3 Hz, ³J_{H,F}=27.8 Hz, 11-CH₂F). Elemental analysis: C₂₄H₃₀F₂N₂O₅S (M=496.57 g/mol), calcd. C, 58.05; H, 6.09; N, 5.64. found C, 57.94, H, 6.24, N, 5.57%. Exact mass (ESI⁺): C₂₄H₃₀F₂N₂O₅S+H⁺, calcd. 497.1922. found 497.1908; C₂₄H₃₀F₂N₂O₅S+Na⁺, calcd. 519.1741. found 519.1733; (C₂₄H₃₀F₂N₂O₅S)₂+Na⁺, calcd. 1015.3585. found 1015.3566. Optical rotation: [α]₅₈₉²⁰=−23.9, [α]₅₇₈²⁰=−25.1, [α]₅₄₆²⁰=−28.9, [α]₄₃₆²⁰=−52.6, [α]₃₆₅²⁰=−92.9 (c=0.993, CHCl₃).

### Example 30b

### Compound MAB 255

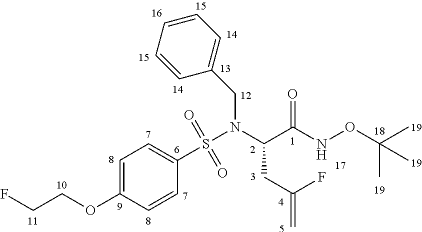

Prepared from N-benzyl-N-[p-(2-fluorethoxy)phenylsulfonyl]-2-amino-4-fluoropent-4-enoic acid (206 mg, 0.483 mmol), HOPT (65 mg, 0.483 mmol), NMM (245 mg, 2.415 mmol), O-tert-butylhydroxylamine (182 mg, 1.450 mmol) and EDC (121 mg, 0.628 mmol). Yield: 200 mg (83%). ¹H NMR (300 MHz, CDCl₃): δ=8.46 (s, 1H, 17-NH), 7.69 (dm, ³J_{H,H}=8.9 Hz, 2H, 8-CH), 7.29 (m, 5H, 14-CH, 15-CH, 16-CH), 6.96 (dm, ³J_{H,H}=9.0 Hz, 2H, 7-CH), 4.78 (dm, ²J_{H,F}=47.4 Hz, 2H, 11-CH₂), 4.64 (d, ²J_{H,H}=15.7 Hz, 1H, 12-CH_{A}), 4.54 (t, ³J_{H,H}=7.2 Hz, 1H, 2-CH), 4.42 (dd, ³J_{H,F}=17.1 Hz, ²J_{H,H}=3.0 Hz, 2H, 5-CH_{cis}), 4.40 (d, ²J_{H,H}=15.6 Hz, 1H, 12-CH_{B}) 4.27 (m, ³J_{H,F}=27.6 Hz, 2H, 10-CH₂), 4.05 (dd, ³J_{H,F}=50.0 Hz, ²J_{H,H}=3.0 Hz, 1H, 5-CHₜᵣₐₙₛ), 2.821 (ddd, ³J_{H,F}=18.5 Hz, ²J_{H,H}=14.9 Hz, ³J_{H,H}=7.6 Hz, 1H, 3-CH_{A}), 2.31 (ddd, ³J_{H,F}=19.1 Hz, ²J_{H,H}=14.9 Hz, ³J_{H,H}=6.9 Hz, 1H, 3-CH_{B}), 1.23 (s, 9H, 19-CH₃). ¹³C NMR (75 MHz, CDCl₃): δ=167.0 (s, C-1), 161.9 (s, C-9), 161.3 (ds, ¹J_{H,F}=255.8 Hz, C-4), 136.5 (s, C-13), 131.9 (s, C-6), 129.6 (d, C-8), 128.8 (d, C-14 o. C-15), 128.6 (d, C-14 o. C-15), 128.0 (d, C-16), 114.8 (d, C-7), 93.7 (dt, ²J_{H,F}=19.2 Hz, C-5), 82.4 (s, C-18), 81.4 (dt, ¹J_{H,F}=171.8 Hz, C-11), 67.4 (dt, ²J_{H,F}=20.5 Hz, C-10), 55.1 (d, C-2), 48.7 (t, C-12), 32.4 (dt, ²J_{C,F}=27.6 Hz, C-3), 26.2 (q, C-19). ¹⁹F NMR (282 MHz, CDCl₃): δ =−98.51 (m, ³J_{H,F}=50.3 Hz, ³J_{H,F}=18.0 Hz, 4-CF), −224.35 (tt, ²J_{H,F}=47.3 Hz, ³J_{H,F}=27.8 Hz, 11-CH₂F). Elemental analysis: C₂₄H₃₀F₂N₂O₅S (M=496.57 g/mol), calcd. C, 58.05; H, 6.09; N, 5.64. found C, 57.78; H, 6.24; N, 5.56%. Exact mass (ESI⁺): C₂₄H₃₀F₂N₂O₅S+FE: calcd. 497.1922. found 497.1917; C₂₄H₃₀F₂N₂O₅S+Na⁺, calcd. 519.1741. found 519.1733; (C₂₄H₃₀F₂N₂O₅S)₂+Na⁺, calcd. 1015.3585. found 1015.3564. Optical rotation: [α]₅₈₉²⁰=+21.7 (c=1.039, CHCl₃).

### Example 31

### Example 31a

### Compound MAB 258

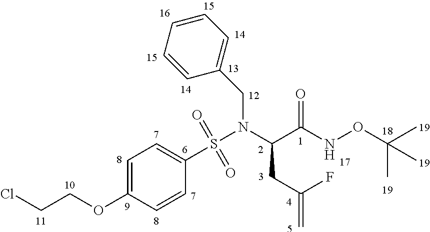

Prepared from N-benzyl-N-[p-(2-chloroethoxy)phenylsulfonyl]-2-amino-4-fluoropent-4-enoic acid (243 mg, 0.55 mmol) HOBT (75 mg, 0.55 mmol), NMM (283 mg, 2.80 mmol) O-tert-butylhydroxylamine (213 mg, 1.70 mmol) and EDC (138 mg, 0.72 mmol). Yield: 204 mg (72%). ¹H NMR (300 MHz, CDCl₃): δ=8.48 (s, 1H, 17-NH), 7.69 (dm, ³J_{H,H}=8.9 Hz, 2H, 8-CH), 7.28 (m, 5H, 14-CH, 15-CH, 16-CH), 6.94 (dm, ³J_{H,H}=8.8 Hz, 2H, 7-CH), 4.64 (d, ²J_{H,H}=15.7 Hz, 1H, 12-CH_{A}), 4.53 (t, ³J_{H,H}=7.2 Hz, 1H, 2-CH), 4.42 (dd, ³J_{H,F}=17.1 Hz, ²J_{H,H}=3.0 Hz, 1H, 5-CH_{cis}), 4.41 (d, ²J_{H,H}=15.5 Hz, 1H, 12-CH_{B}), 4.28 (t, ³J_{H,H}=5.7 Hz, 2H, 10-CH₂), 4.06 (dd, ³J_{H,F}=49.9 Hz, ²J_{H,H}=3.1 Hz, 1H, 5-CHₜᵣₐₙₛ), 3.84 (t, J=5.7 Hz, 2H, 11-CH₂), 2.82 (ddd, ³J_{H,F}=18.5 Hz, ²J_{H,H}=14.9 Hz, ³J_{H,H}=7.6 Hz, 1H, 3-CH_{A}), 2.31 (ddd, ³J_{H,F}=18.9 Hz, ²J_{H,H}=14.9 Hz, ³J_{H,H}=6.9 Hz, 1H, 3-CH_{B}), 1.23 (s, 9H, 19-CH₃). ¹³C NMR (75 MHz, CDCl₃): δ=167.0 (s, C-1), 161.8 (s, C-9), 161.3 (ds, ¹J_{H,F}=255.8, C-4), 136.4 (s, C-13), 131.9 (s, C-6), 129.7 (d, C-8), 128.8 (d, C-14 o. C-15), 128.6 (d, C-14 o. C-15), 127.9 (d, C-16), 114.8 (d, C-7), 93.7 (dt, ²J_{C,F}=19.4, C-5), 82.4 (s, C-18), 68.2 (t, C-10), 55.0 (d, C-2), 48.7 (t, C-12), 41.4 (t, C-11), 32.4 (dt, ²J_{H,F}=27.6 Hz, C-3), 26.1 (q, C-19). ¹⁹F NMR (282 MHz, CDCl₃): δ =−98.49 (m, ³J_{H,F}=49.9 Hz, ³J_{H,F}=18.9 Hz, ³J_{H,F}=17.2 Hz 4-CF). Elemental analysis: C₂₄H₃ClFN₂O₅S (M=513.02 g/mol), calcd. C, 56.19; H, 5.89; N, 5.46. found C, 55.55; H, 5.89; N, 5.34%. Exact mass (ESI⁺): C₂₄H₃₀ClFN₂O₅S+Na⁺, calcd. 535.1446. found 535.1434. Optical rotation: [α]₅₈₉²⁰=−2.2, [α]₃₆₅²⁰=−6.9 (c=1.025 CHCl₃).

### Example 31b

### Compound MAB 259

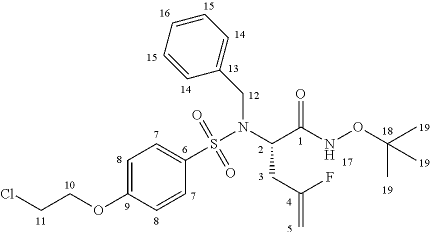

Prepared from N-benzyl-N-[p-(2-chloroethoxy)phenylsulfonyl]-2-amino-4-fluoropent-4-enoic acid (348 mg, 0.79 mmol), HOBT (107 mg, 0.79 mmol), NMM (405 mg, 3.94 mmol), O-tert-butylhydroxylamine (305 mg, 2.36 mmol) and EDC (198 mg, 1.20 mmol). Yield: 302 mg (75%). ¹H NMR (300 MHz, CDCl₃): δ=8.48 (s, 1H, 17-NH), 7.69 (dm, ³J_{H,H}=8.9 Hz, 2H, 8-CH), 7.28 (m, 5H, 14-CH, 15-CH, 16-CH), 6.94 (dm, ³J_{H,H}=8.9 Hz, 2H, 7-CH), 4.64 (d, ²J_{H,H}=15.6 Hz, 1H, 12-CH_{A}), 4.53 (t, ³J_{H,H}=7.2 Hz, 1H, 2-CH), 4.43 (dd, ³J_{H,F}=17.1 Hz, ²J_{H,H}=3.0 Hz, 1H, 5-CH_{cis}), 4.40 (d, ²J_{H,H}=15.6 Hz, 1H, 12-CH_{B}), 4.28 (t, ³J_{H,H}=5.7 Hz, 2H, 10-CH₂), 4.06 (dd, ³J_{H,F}=49.6 Hz, ²J_{H,H}=3.5 Hz, 1H, 5-CHₜᵣₐₙₛ), 3.84 (t, J=5.7 Hz, 2H, 11-CH₂), 2.82 (ddd, ³J_{H,F}=18.6 Hz, ²J_{H,H}=14.9 Hz, ³J_{H,H}=7.6 Hz, 1H, 3-CH_{A}), 2.31 (ddd, ³J_{H,F}=18.9 Hz, ²J_{H,H}=14.9 Hz, ³J_{H,H}=6.8 Hz, 1H, 3-CH_{B}), 1.23 (s, 9H, 19-CH₃). ¹³C NMR (75 MHz, CDCl₃): δ=167.0 (s, C-1), 161.8 (s, C-9), 161.3 (ds, ¹J_{H,F}=256.1, C-4), 136.4 (s, C-13), 131.9 (s, C-6), 129.7 (d, C-8), 128.8 (d, C-14 o. C-15), 128.6 (d, C-14 o. C-15), 127.9 (d, C-16), 114.8 (d, C-7), 93.7 (dt, ²J_{C,F}=19.2, C-5), 82.4 (s, C-18), 68.2 (t, C-10), 55.0 (d, C-2), 48.7 (t, C-12), 41.4 (t, C-11), 32.4 (dt, ²J_{H,F}=27.1 Hz, C-3), 26.1 (q, C-19). ¹⁹F NMR (282 MHz, CDCl₃): δ =−98.50 (m, ³J_{H,F}=49.9 Hz, ³J_{H,F}=18.5 Hz 4-CF). Elemental analysis: C₂₄H₃₀ClFN₂O₅S (M=513.02 g/mol), calcd. C, 56.19; H, 5.89; N, 5.46. found C, 55.20, H, 5.88, N, 5.36%. Exact mass (ESI⁺): C₂₄H₃₀ClFN₂O₅8 +Na⁺, calcd. 535.1446. found 535.1434. Optical rotation: [α]₅₈₉²⁰=+21.5, [α]₅₇₈²⁰=+22.2, [α]₅₄₆²⁰=+25.5, [α]₄₃₆²⁰=+46.5, [α]₃₆₅²⁰=+82.2 (c=0.999, CHCl₃).

General Procedure for the Preparation of Tetrahydropyranyl (THP)-Protected Hydroxamic Acids

To a solution of carboxylic acid (100 mg, 0.235 mmol) in DMF (0.06 mmol/mL, 1 mL) 1-hydroxybenzotriazole hydrate (HOBT, 1.2 eq., 38 mg, 0.282 mmol), 4-methylmorpholine (NMM, 3.0 eq., 78 μL, 249.70 mmol), O-tetrahydro-2H-pyran-2-yl-hydroxylamine (3.1 eq., 85 mg, 0.729 mmol and N-[dimethylamino)-propyl]-N′-ethylcarbodiimide hydrochloride (EDC, 1.4 eq., 63 mg, 0.329 mmol) were added. After stirring overnight at room temperature the reaction mixture was diluted with water (20 mL) and extracted with ethyl acetate (3×5 mL). The combined organic phases were washed successively with water, 5% aqueous KHSO₄, saturated aqueous NaHCO₃ and brine, and dried over magnesium sulfate. After removing the solvent under reduced pressure column chromatographic purification (silica gel, cyclohexane/ethyl acetate 2:1) yielded the THP-protected hydroxamic acid.

### Example 32

### Example 32a

### (2S)-2-[N-benzyl-4-(2-fluoroethoxy)phenylsulfonamido]-4-fluoro-N-[(tetrahydro-2H-pyran-2-yl)oxy]pent-4-enamide (HUG 72)

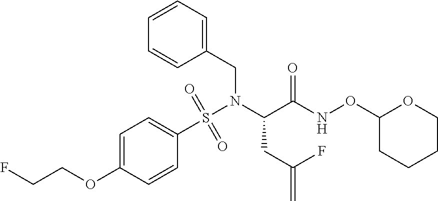

Light brown wax; yield: 92 mg (75%). ¹H NMR (300 MHz, CDCl₃); δ 9.19 (s, NH, 1H), 9.15 (s, NH, 1H), 7.77 (d, ³J_{H,H}=8.9 Hz, PhH, 2H), 7.72 (d, ³J_{H,H}=9.0 Hz, PhH, 2H), 7.39-7.21 (m, PhH, 5H), 6.98 (d, ³J_{H,H}=9.0 Hz, PhH, 2H), 6.97 (d, ³J_{H,H}=9.0 Hz, PhH, 2H), 4.92-4.88 (m, NHOCHO), 4.79 (dm, ²J_{H,F}=47.4 Hz, CH₂F, 2H), 4.66 (AB, d, ²J_{H,H}=16.0 Hz, NCH₂, 1H), 4.58-4.45 (m, NCH, NCH₂, 2H), 4.43 (dd, CFCH₂, H_{cis}, ³J_{H,F}=17.1 Hz, ²J_{H,F}=3.0 Hz, 1H), 4.39 (dd, CFCH₂, H_{cis}, ³J_{H,F}=17.1 Hz, ²J_{H,F}=3.0 Hz, 1H), 4.27 (dm, ³J_{H,F}=27.7 Hz, O—CH₂CH₂F, 2H), 4.10 (dd, CFCH₂, Hₜᵣₐₙₛ, ³J_{H,F}=49.9 Hz, 2J_{H,F}=3.1 Hz, 1H), 3.95-3.84 (m, NHOCHOCH₂, 1H), 3.70-3.55 (m, NHOCHOCH₂, 1H), 2.93-2.58 (AB, m, NCHCH₂, 1H), 2.47-2.18 (AB, m, NCHCH₂, 1H), 1.88-1.50 (m, THP-CH₂, 6H). ¹³C NMR (75 MHz, CDCl₃): δ 165.8 (CONH), 165.6 (CONH), 162.0 (q, PhCOCH₂CH₂F), 161.2 (d, ¹J_{H,F}=256.3 Hz, CFCH₂), 161.2 (d, ¹J_{H,F}=256.0 Hz, CFCH₂), 136.6 (qPhCCH₂N), 136.4 (qPhCCH₂N), 131.5 (qPhCSO₂), 129.7 (PhCH), 129.7 (PhCH), 128.7 (PhCH), 128.6 (PhCH), 128.5 (PhCH), 128.4 (PhCH), 127.8 (PhCH), 127.8 (PhCH), 114.7 (PhCH), 114.7 (PhCH), 102.1 (NHOCHO), 101.7 (NHOCHO), 93.7 (d, ²J_{H,F}=19.0 Hz, CFCH₂), 93.6 (d, ²J_{H,F}=18.9 Hz, CFCH₂), 81.4 (¹J_{H,F}=171.6 Hz, OCH₂CH₂F), 67.4 (d, ²J_{H,F}=20.5 Hz, OCH₂CH₂F), 62.0 (NHOCHOCH₂), 54.7 (NCH), 54.5 (NCH), 48.6 (NCH₂), 48.5 (NCH₂), 32.3 (d, ²J_{C,F}=27.6 Hz, NCHCH₂CF), 32.0 (d, ²J_{H,F}=26.9 Hz, NCHCH₂CF), 27.7 (NHOCHCH₂), 27.7 (NHOCHCH₂), 24.9 (CH₂), 18.2 (NHOCHCH₂CH₂). MS-ES-EM: m/z=547.1683 [M+Na)⁺] calcd for C₂₅H₃₀F₂N₂O₆SNa⁺: 547.1685.

### Example 32b

### (2R)-2-[N-benzyl-4-(2-fluoroethoxy)phenylsulfonamido]-4-fluoro-N-[(tetrahydro-2H-pyran-2-yl)oxy]pent-4-enamide (HUG 71)

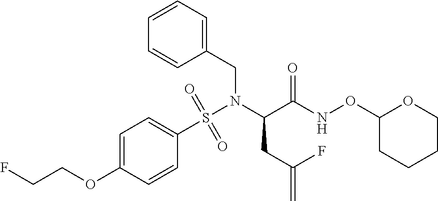

Light brown wax; yield: 101 mg (81%). ¹H NMR (300 MHz, CDCl₃): δ 9.08 (s, NH, 1H), 7.77 (d, ³J_{H,H}=9.0 Hz, PhH, 2H), 7.72 (d, ³J_{H,H}=9.0 Hz, PhH, 2H), 7.39-7.21 (m, PhH, 5H), 6.98 (d, ³J_{H,H}=9.0 Hz, PhH, 2H), 6.97 (d, ³J_{H,H}=9.0 Hz, PhH, 2H), 4.92-4.88 (m, NHOCHO), 4.80 (dm, ²J_{H,F}=47.3 Hz, CH₂F, 2H), 4.66 (AB, m, NCH₂, 1H), 4.58-4.45 (m, NCH, NCH₂, 2H), 4.45-4.34 (m, CFCH₂, H_{cis}, 1H), 4.28 (dm, ³J_{H,F}=27.7 Hz, O—CH₂CH₂F, 2H), 4.10 (dd, CFCH₂, Hₜᵣₐₙₛ, ³J_{H,F}=49.9 Hz, ²J_{H,F}=3.0 Hz, 1H), 3.95-3.84 (m, NHOCHOCH₂, 1H), 3.70-3.58 (m, NHOCHOCH₂, 1H), 2.92-2.64 (AB, m, NCHCH₂, 1H), 2.43-2.18 (AB, m, NCHCH₂, 1H), 1.88-1.50 (m, THP-CH₂, 6H). ¹³C NMR (75 MHz, CDCl₃): δ 165.8 (CONH), 165.7 (CONH), 162.0 (qPhCOCH₂CH₂F), 161.3 (d, ¹J_{H,F}=256.1 Hz, CFCH₂), 136.6 (qPhCCH₂N), 136.4 (qPhCCH₂N), 131.6 (qPhCSO₂), 129.8 (PhCH), 129.8 (PhCH), 128.8 (PhCH), 128.7 (PhCH), 128.5 (PhCH), 128.5 (PhCH), 127.9, (PhCH), 127.9 (PhCH), 114.8 (PhCH), 114.8 (PhCH), 102.2 (NHOCHO), 101.8 (NHOCHO), 93.8 (d, ²J_{C,F}=18.9 Hz, CFCH₂), 81.4 (¹J_{C,F}=171.9 Hz, OCH₂CH₂F), 67.4 (d, ²J_{H,F}=20.5 Hz, OCH₂CH₂F), 62.1 (NHOCHOCH₂), 62.1 (NHOCHOCH₂), 54.7 (NCH), 54.6 (NCH), 48.7 (NCH₂), 48.5 (NCH₂), 32.2 (d, ²J_{H,F}=27.6 Hz, NCHCH₂CF), 27.7 (NHOCHCH₂), 24.9 (CH₂), 18.2 (NHOCHCH₂CH₂). ¹⁹F NMR (282 MHz, CDCl₃): δ −97.51 and 97.77 (m, CFCH₂, 1F), −223.89 and 223.90 (tt, ²J_{H,F}=47.3, ³J_{H,F}=27.6 Hz, OCH₂CH₂F, 1F). MS-ES-EM m/z=547.1695 [M+Na)⁺] calcd for C₂₅H₃₀F₂N₂O₆SNa⁺: 547.1685.

General Procedure for the Acidic Hydrolysis of the THP-Protected Hydroxamic Acids

To a solution of THP-protected hydroxamic acid in 1,4-dioxane (0.5 mL/0.18 mmol) 4 N hydrochloric acid in 1,4-dioxane (4 eq.) and methanol (0.5 mL/0.18 mmol) were added and the reaction mixture was stirred at room temperature. The reaction progress was monitored by TLC (ethyl acetate). After complete conversion the reaction mixture was diluted with ethyl acetate (20 mL), washed with water (3×10 mL) and dried (MgSO₄). The solvent was removed under reduced pressure and the product was purified by column chromatography (silica gel, cyclohexane/ethyl acetate 2:1).

### Example 33

### Example 33a

### (S)-2-[N-benzyl-4-(2-fluoroethoxy)phenylsulfonamido]-4-fluoro-N-hydroxypent-4-enamide (HUG 74)

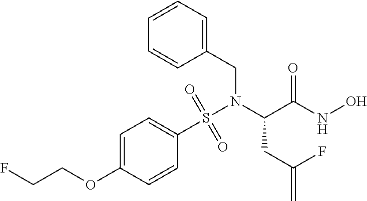

The reaction was carried out in a 0.175 mmol scale with (2S)-2-(N-benzyl-4-(2-fluoroethoxy)phenylsulfonamido)-4-fluoro-N-[(tetrahydro-2H-pyran-2-yl)oxy]pent-4-enamide (HUG 72). After column chromatography a light brown wax was obtained. Yield: 33 mg (43%). ¹H NMR (400 MHz, CDCl₃): δ 9.19 (s, 1 OH or NH), 7.72 (d, ³J_{H,H}=8.9 Hz, PhH, 2H), 7.35-7.25 (m, PhH, 5H), 6.97 (d, ³J_{H,H}=8.9 Hz, PhH, 2H), 4.79 (dm, ²J_{H,F}=47.3 Hz, CH₂F, 2H), 4.54 (AB, d, ²J_{H,H}=15.6 Hz, NCH₂, 1H), 4.52 (m, NCH, 1H), 4.46 (AB, d, ²J_{H,H}=15.7 Hz, NCH₂, 1H), 4.38 (dd, CFCH₂, H_{cis}, ³J_{H,F}=17.0 Hz, ²J_{H,F}=2.9 Hz, 1H), 4.26 (dm, ³J_{H,F}=27.6 Hz, CH₂FCH₂, 2H), 4.08 (dd, CFCH₂, Hₜᵣₐₙₛ, ³J_{H,F}=49.7 Hz, ²J_{H,F}=3.1 Hz, 1H), 2.85-2.69 (AB, m, NCHCH₂, 1H), 2.38 (AB, m, NCHCH₂, 1H). ¹³C NMR (101 MHz, CDCl₃): δ 166.4 (CONH), 162.1 (qPhCOCH₂CH₂F), 161.1 (d, ¹J_{H,F}=256.3 Hz, CFCH₂), 136.1 (qPhCCH₂N), 131.3 (qPhCSO₂), 129.7 (PhCH), 128.6 (PhCH), 128.6 (PhCH), 128.0 (PhCH), 114.8 (PhCH), 93.8 (d, ²J_{H,F}=18.9 Hz, CFCH₂), 81.4 (d, ¹J_{C,F}=171.7 Hz, OCH₂CH₂F), 67.4 (d, ²J_{H,F}=20.4 Hz, OCH₂CH₂F), 54.2 (NCH), 48.7 (NCH₂), 31.7 (d, ²J_{C,F}=27.7 Hz, NCHCH₂CF). ¹⁹F NMR (282 MHz, CDCl₃): δ −97.58 (m, CFCH₂, 1F), −223.84 (tt, ²J_{H,F}=47.3, ³J_{H,F}=27.6 Hz, OCH₂CH₂F, 1F). MS-ES-EM: m/z=463.11201[(M+Na)⁺] calcd for C₂₀H₂₂F₂N₂O₅SNa⁺: 463.1110. HPLC t_{R}=9.05 min (100%).

### Example 33b

### (R)-2-[N-benzyl-4-(2-fluoroethoxy)phenylsulfonamido]-4-fluoro-N-hydroxypent-4-enamide (HUG 78)

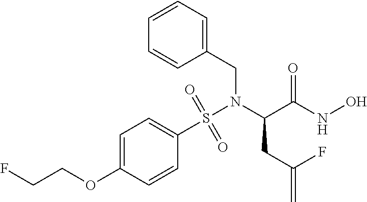

The reaction was carried out in a 0.190 mmol scale with (2R)-2-[N-benzyl-4-(2-fluoroethoxy)phenylsulfonamido]-4-fluoro-N-[(tetrahydro-2H-pyran-2-yl)oxy]pent-4-enamide (HUG 71). After column chromatography a light brown wax was obtained. Yield: 52 mg (62%). ¹H NMR (400 MHz, CDCl₃): δ 9.25 (s, 1OH or NH), 7.71 (d, ³J_{H,H}=8.8 Hz, PhH, 2H), 7.38-7.18 (m, PhH, 5H), 6.96 (d, ³J_{H,H}=8.8 Hz, PhH, 2H), 4.79 (dm, ²J_{H,F}=47.2 Hz, CH₂F, 2H), 4.54 (AB, d, ²J_{H,H}=15.5 Hz, NCH₂, 1H), 4.52 (m, NCH, 1H), 4.46 (AB, d, ²J_{H,H}=15.8 Hz, NCH₂, 1H), 4.38 (dd, CFCH₂, H_{cis}, ³J_{H,F}=17.0 Hz, ²J_{H,F}=2.8 Hz, 1H), 4.26 (dm, ³J_{H,F}=27.3 Hz, CH₂FCH₂, 2H), 4.08 (dd, CFCH₂, H trans, ³J_{H,F} 50.2 Hz, ²J_{H,F}=2.2 Hz, 1H), 2.86-2.63 (AB, m, NCHCH₂, 1H), 2.47-2.25 (AB, m, NCHCH₂, 1H). ¹³C NMR (101 MHz, CDCl₃): δ 166.4 (CONH), 162.1 (qPhCOCH₂CH₂F), 161.1 (d, ¹J_{H,F}=256.3 Hz, CFCH₂), 136.2 (qPhCCH₂N), 131.3 (qPhCSO₂), 129.7 (PhCH), 128.6 (PhCH), 128.6 (PhCH), 128.0 (PhCH), 114.8 (PhCH), 93.7 (d, ²J_{H,F}=19.4 Hz, CFCH₂), 81.4 (d, ¹J_{H,F}=171.6 Hz, OCH₂CH₂F), 67.4 (d, ²J_{H,F}=20.4 Hz, OCH₂CH₂F), 54.2 (NCH), 48.7 (NCH₂), 31.8 (d, ²J_{C,F}=27.8 Hz, NCHCH₂CF). ¹⁹F NMR (282 MHz, CDCl₃): δ −97.54 (ddd, ³J_{H,F}=49.9 Hz, ³J_{H,F}=36.9 Hz, ³J_{H,F}=17.1 Hz, CFCH₂, 1F), −223.83 (tt, ²J_{H,F}=47.3, ³J_{H,F}=27.6 Hz, OCH₂CH₂F, 1F). MS-ES-EM: m/z=463.11049 [(M+Na)⁺] calcd for C₂₀H₂₂F₂N₂O₅SNa⁺: 463.11097. HPLC t_{R}=9.10 min (99%).

### Example 34

### Assay for MMP-2 and MMP-9 Inhibitory Activity

The synthetic broad-spectrum fluorogenic substrate (7-methoxycoumarin-4-yl)acetyl pro-Leu-Gly-Leu-(3-(2,4-dinitrophenyl)-L-2,3-diamino-propionyl)-Ala-Arg-NH₂ (R & D Systems) was used to assay MMP-2 and MMP-9 activity.

The inhibition of MMP-2 and MMP-9 by the MMP inhibitors of table 1 was assayed by incubating either MMP-2 (2 nM) or MMP-9 (2 nM) and the compounds to be assayed at different concentrations (10 pM-1 mM) for 30 mM at 37° C. in 50 mM Tris.HCl (pH=7.5), 0.2M NaCl, 5 mM CaCl₂, 20 μM ZnSO₄ and 0.05% Brij35.

An aliquot of substrate (10 μI of a 50 μM solution) was then added to 90 μM of pre-incubated MMP/compound mixture, and activity was determined at 37° C. by following product release with time. The fluorescence changes were monitored using a Fusion Universal Microplate Analyzer (Packard Bioscience) with excitation and emission wavelengths set to 330 and 390 nm, respectively. Reaction rates were measured from the initial 10 mM of the reaction profile where product release was linear with time and plotted as a function of inhibitor dose. From the resulting inhibition curves, the IC₅₀ values for each inhibitor were calculated by nonlinear regression analysis, performed using the GRACE 5.1.8 software (Linux).

TABLE 1
IC₅₀ values obtained for the compounds of formula (I) assayed and comparative
compounds CGS-27023A and (S)-CGS-27023A.

| Compounds (S) | MMP-2 | MMP-9 | Compounds (R) | MMP-2 | MMP-9 |
|---|---|---|---|---|---|
| 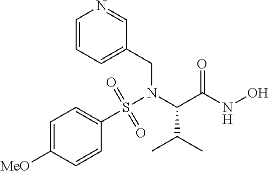 (S)-CGS-27023A | 494 nM¹⁾ | 198 nM¹⁾ | 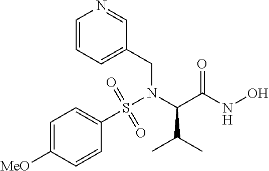 CGS-27023A | 20 nM | 8 nM |
| 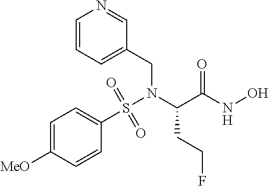 (S)-22a | 32.8 nM ± 16.4 nM | 3.0 nM ± 0.7 nM | 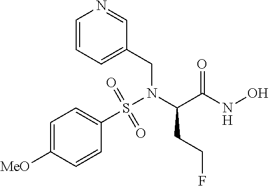 (R)-22b | 6.4 nM ± 1.8 nM | 12.3 nM ± 1.0 nM |
| 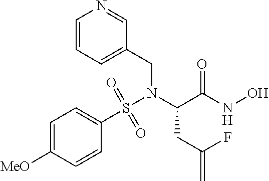 (S)-23a | 7.2 nM ± 0.1 nM | 4.9 nM ± 0.4 nM | 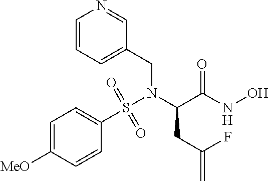 (R)-23b | 9.3 nM ± 2.0 nM | 8.3 nM ± 0.1 nM |
| 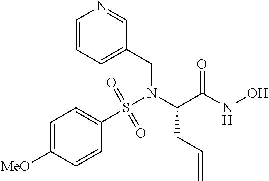 (S)-21a | 6.3 nM ± 1.5 nM | 6.4 nM ± 0.4 nM | 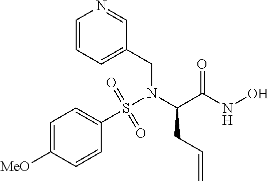 (R)-21b | 3.9 nM ± 0.3 nM | 0.54 nM ± 0.13 nM |
| 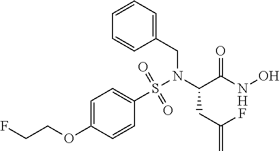 (S)-33a (HUG 74) | 22.2 nM | 6.96 nM | 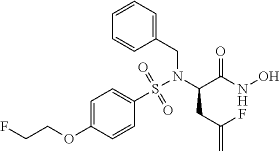 (R)-33b (HUG 78) | 10.4 nM | 2.86 nM |

¹⁾calculated from the Kᵢ values

As can be observed from the data reported in Table 1, the compounds of the invention show an improved activity with respect to the reference compounds CGS-27023A. This effect is remarkable also in the (S)-enantiomer of the compounds of the invention.

Having thus descried in detail advantageous embodiments of the present invention, it is to be understood that the invention defined by the above paragraphs is not to be limited to particular details set forth in the above description as many apparent variations thereof are possible without departing from the spirit or scope of the present invention.

We claim:

1. A compound of formula (I): 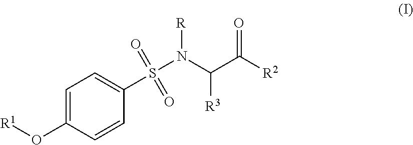
wherein:
R is an optionally substituted arylalkyl or an optionally substituted heteroarylalkyl;
R¹ is (CH₂—CH₂—O—)ₙ—CH₂—CH₂—R⁵, C₁-C₆ alkyl, C₁-C₆ alkenyl or C₁-C₇ alkynyl optionally substituted with one or more substituents selected from halogen, OH or a leaving group;
R² is C₁-C₆ alkyl, C₁-C₆ alkoxy or —NH—OR⁴;
R³ is C₁-C₆ fluoro-alkyl or a C₂-C₆ alkenyl optionally substituted with a halogen or a radionuclide;
R⁴ is H, C₁-C₆ alkyl, C₃-C₆ cycloalkyl or C₅-C₆ heterocycloalkyl;
R⁵ is F, Cl, OH, or a leaving group; and
n is 0, 1, 2, 3, 4, 5 or 6;
and enantiomers, esters and pharmaceutically acceptable salts thereof.

2. The compound according to claim 1 wherein:
R is pyrid-3-yl-CH₂— or phenyl-CH₂—;
R¹ is —CH₃, —CH₂—CH₂Cl, —CH₂—CH₂F, —CH₂—CH₂₋OTs or —CH₂—CH₂OH;
R² is —NH—OR⁴;
R³ is —CH₂—CH₂F, —CH₂—C(F)═CH₂ or CH₂—CH═CH₂; and
R⁴ is H, methyl, tert-butyl or tetrahydropyranyl.

3. The compound according to claim 1 wherein the carbon atom bearing radical R³ has an “S” configuration.

4. The compound according to claim 1 wherein the carbon atom bearing radical R³ has an “R” configuration.

5. A compound selected from the group consisting of: 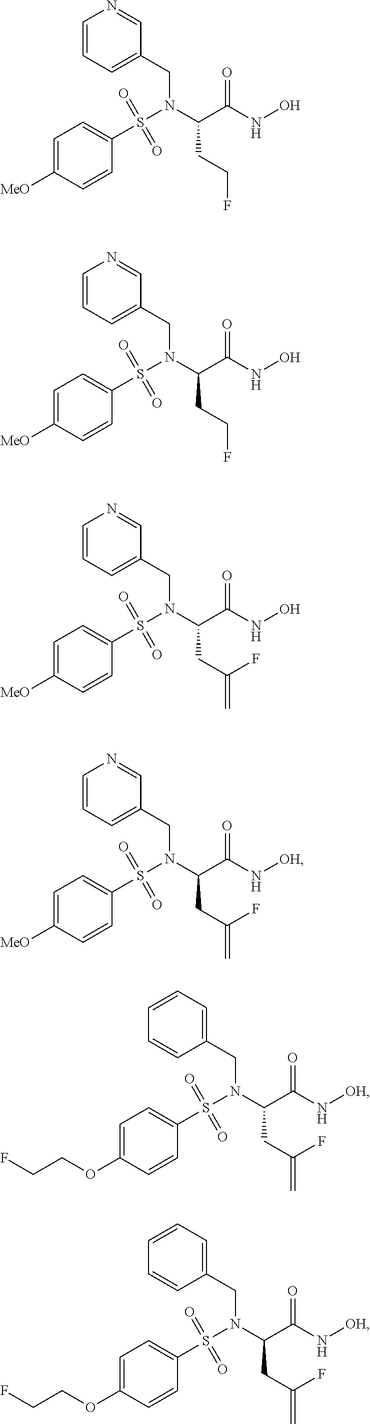 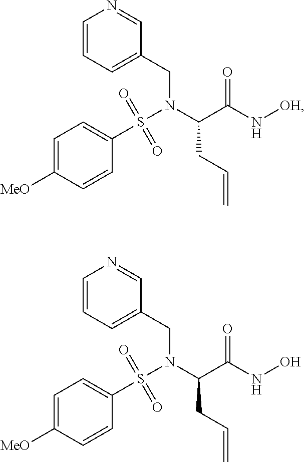
racemates, esters and pharmaceutically acceptable salts thereof.

6. The compound of claim 1 wherein at least one carbon atom bears an ¹⁸F in addition to or in place of a substituent already present on said carbon atom.

7. The compound according to claim 6 wherein the carbon atom is the carbon atom of radical R³.

8. The compound according to claim 7 wherein R³ is C₁-C₆ (¹⁸F) alkyl or C₂-C₆ (¹⁸F)-alkenyl.

9. The compound according to claim 8 wherein R³ is CH₂—CH₂¹⁸F or CH₂—C(¹⁸F)═CH₂.

10. The compound according to claim 6 wherein the carbon atom is the carbon atom of radical R¹.

11. The compound according to claim 10, wherein R¹ is —CH₂—CH₂¹⁸F.

12. A compounds selected from the group consisting of: 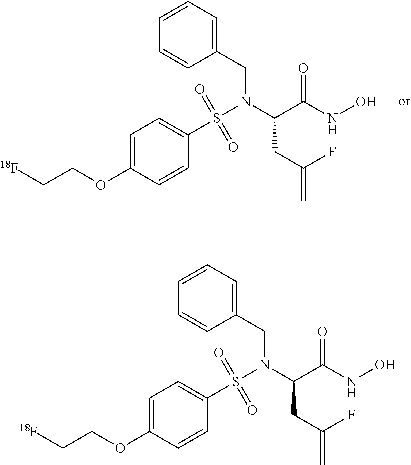
racemate, esters and pharmaceutically acceptable salts thereof.

13. A method for imaging a biological target, the method comprising:
(a) administering a compound of formula (I) or a racemate, ester or pharmaceutically acceptable salts thereof, to a human or animal comprising a biological target, 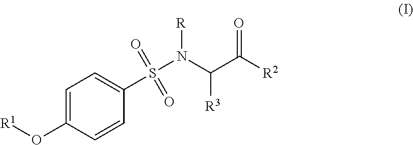
wherein:
R is an optionally substituted arylalkyl or an optionally substituted heteroarylalkyl;
R¹ is (CH₂—CH₂—O—)ₙ—CH₂—CH₂—R⁵, C₁-C₆ alkyl, C₁-C₆ alkenyl or C₁-C₇ alkynyl optionally substituted with one or more substituents selected from halogen, OH or a leaving group;
R² is C₁-C₆ alkyl, C₁-C₆ alkoxy or —NH—OR⁴;
R³ is C₁-C₆ fluoro-alkyl or a C₂-C₆ alkenyl;
R⁴ is H, C₁-C₆ alkyl, C₃-C₆ cycloalkyl or C₅-C₆ heterocycloalkyl;
R⁵ is F, Cl, OH, or a leaving group; and
n is 0, 1, 2, 3, 4, 5 or 6,
wherein at least one carbon atom bears a radionuclide in addition to or in place of a substituent already present on said carbon atom; and
(b) imaging the compound.

14. The method according to claim 13, wherein the imaging is performed on a PET or SPECT machine.

15. The method according to claim 13 wherein the target is associated with an unpaired expression of matrix-metalloprotease.

16. A method of treating pathological conditions associated with an unpaired expression of matrix-metalloprotease in human and animal, the method comprising:
administering to the human or animal a compound of formula (I) 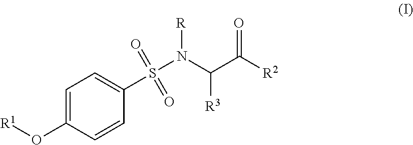
wherein:
R is an optionally substituted arylalkyl or an optionally substituted heteroarylalkyl;
R¹ is (CH₂—CH₂—O—)ₙ—CH₂—CH₂—R⁵, C₁-C₆ alkyl, C₁-C₆ alkenyl or C₁-C₇ alkynyl optionally substituted with one or more substituents selected from halogen, OH or a leaving group;
R² is C₁-C₆ alkyl, C₁-C₆ alkoxy or —NH—OR⁴;
R³ is C₁-C₆ fluoro-alkyl or a C₂-C₆ alkenyl optionally substituted with a halogen;
R⁴ is H, C₁-C₆ alkyl, C₃-C₆ cycloalkyl or C₅-C₆ heterocycloalkyl;
R⁵ is F, Cl, OH, or a leaving group;
n is 0, 1, 2, 3, 4, 5 or 6;
and enantiomers, esters and pharmaceutically acceptable salts thereof.

17. The method according to claim 16 wherein the pathological condition is selected from the group consisting of cardiovascular diseases, inflammatory diseases and malignant diseases.

18. The method according to claim 17 wherein the cardiovascular diseases are selected from atherosclerosis and congestive heart failure and the inflammatory disease is chronic obstructive pulmonary disease.

19. A pharmaceutical composition comprising:
the compound of claim 1 and
a pharmaceutically acceptable carrier.

20. A process for preparing the compound of claim 6, the process comprising:
a) reacting a compound of formula (II) 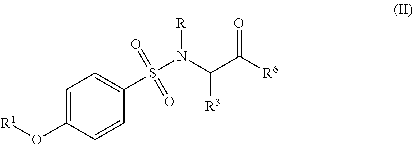
wherein:
R¹ is C₁-C₆ alkyl optionally substituted with OHor a leaving groupnucleophilic substitution;
R³ is C₁-C₆-alkyl or C₂-C₆ alkenyl, each optionally substituted with OH, or a leaving group for nucleophilic substitution with an ¹⁸F containing reagentnucleophilic substitution;
wherein at least one of R¹ and R³ is substituted with OH, a leaving group for nucleophilic substitution
wherein when R¹ is substituted with OH, or a leaving group for nucleophilic substitution, R³ is C₁-C₆ fluoro alkyl or C₂-C₆ alkenyl optionally substituted with F.

21. The process according to claim 20 wherein R¹ is CH₂—CH₂-OTs and R³ is CH₂—CH₂F, CH₂—C(F)═CH₂ or CH₂—CH═CH₂, or R¹ is CH₃ and R³ is CH₂—CH₂OTs.

22. The process according to claim 20 wherein the ¹⁸F containing reagent is [¹⁸F](Kryptofix222)KF.

23. A compound of formula (III): 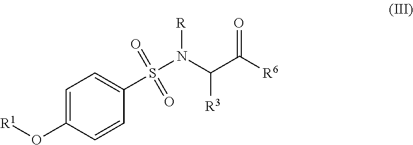
wherein
R is an optionally substituted arylalkyl or an optionally substituted heteroarylalkyl;
R¹ is (CH₂—CH₂—O—)ₙ—CH₂—CH₂—R⁵, C₁-C₆ alkyl, C₁-C₆ alkenyl or C₁-C₇ alkynyl optionally substituted with one or more substituents selected from halogen, OH or a leaving group;
R³ is C₁-C₆ fluoro-alkyl or a C₂-C₆ alkenyl optionally substituted with a halogen or a radionuclide; and
R⁶ is OH.

24. The compound according to claim 23 selected from the group consisting of: 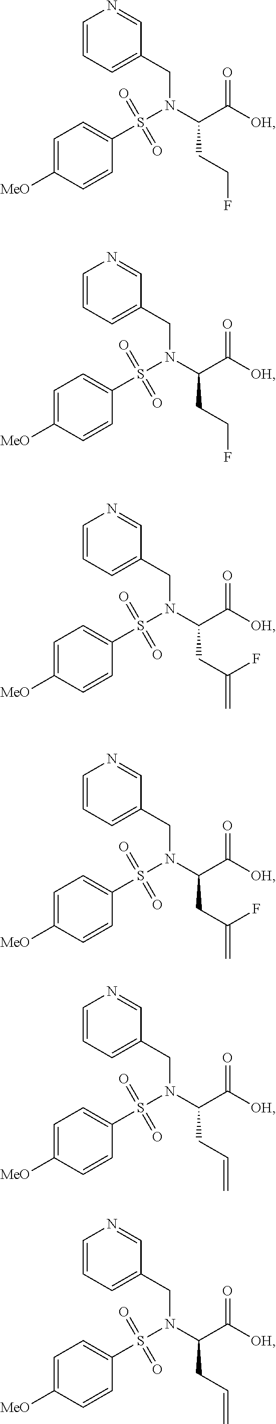 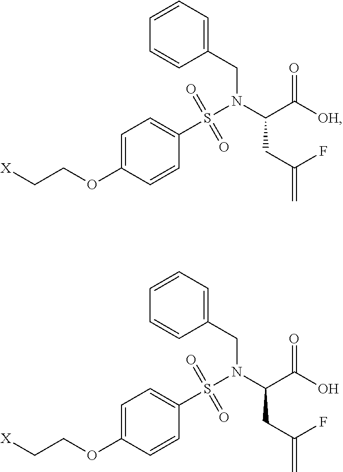
wherein X is F, Cl, OH, a radionuclide or a leaving group.

25. A process for preparing a compound of formula (I): 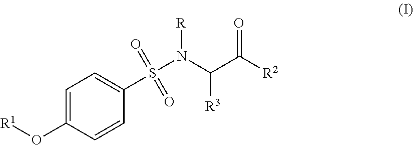
wherein
R is an optionally substituted arylalkyl or an optionally substituted heteroarylalkyl;
R¹ is (CH₂—CH₂—O—)ₙ—CH₂—CH₂—R⁵, C₁-C₆ alkyl, C₁-C₆ alkenyl or C₁-C₇ alkynyl optionally substituted with one or more substituents selected from halogen, OH or a leaving group;
R² is NH—OR⁴;
R³ is C₁-C₆ fluoro-alkyl or a C₂-C₆ alkenyl optionally substituted with a halogen or a radionuclide; and
R⁴ is H, C₁-C₆ alkyl, C₃-C₆ cycloalkyl or C₅-C₆ heterocycloalkyl the process comprising:
a) reacting a compound of formula (III′): 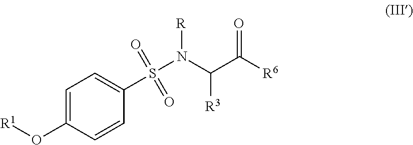
wherein
R is an optionally substituted arylalkyl or an optionally substituted heteroarylalkyl;
R¹ is (CH₂—CH₂—O—)ₙ—CH₂—CH₂—R⁵, C₁-C₆ alkyl, C₁-C₆ alkenyl or C₁-C₇ alkynyl optionally substituted with one or more substituents selected from halogen, OH or a leaving group;
R³ is C₁-C₆ fluoro-alkyl or a C₂-C₆ alkenyl optionally substituted with a halogen or a radionuclide;
and R⁶ is OH or C₁-C₆ alkoxy;
with an hydroxylamine derivative of formula (IV):
R⁴O—NH₂ (IV)
wherein R⁴ is H, C₁-C₆ alkyl, C₃-C₆ cycloalkyl or C₅-C₆ heterocycloalkyl; and
b) when in the compound obtained in step a), R⁴ is C₁-C₆ alkyl, C₃-C₆ cycloalkyl or C₅-C₆ heterocycloalkyl, optionally hydrolyzing it.

26. A process for preparing a compound of formula (III′) 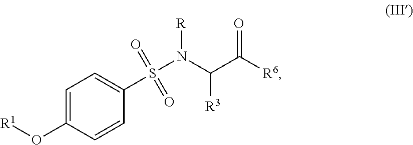
the process comprising:
a′) reacting a compound of formula (V): 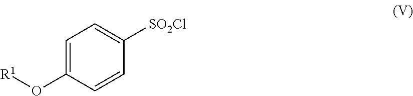
with a compound of formula (VI): 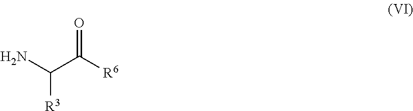
to obtain a compound of formula (VII): 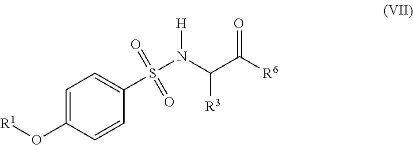
and
a″) reacting the compound of formula (VII) with a compound of formula (VIII):
R—Cl (VIII)
to obtain a compound of formula (III′): 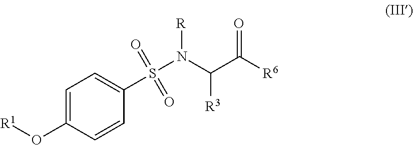
wherein in any of the formula (III′), (V), (VI), and (VII) and (VIII)
R is an optionally substituted arylalkyl or an optionally substituted heteroarylalkyl;
R¹ is (CH₂—CH₂—O—)ₙ—CH₂—CH₂—R⁵, C₁-C₆ alkyl, C₁-C₆ alkenyl or C₁-C₇ alkynyl optionally substituted with one or more substituents selected from halogen, OH or a leaving group;
R² is NH—OR⁴;
R³ is C₁-C₆ fluoro-alkyl or a C₂-C₆ alkenyl optionally substituted with a halogen or a radionuclide;
R⁴ is H, C₁-C₆ alkyl, C₃-C₆ cycloalkyl or C₅-C₆ heterocycloalkyl; and
R⁶ is OH or C₁-C₆ alkoxy.

27. The method of claim 13, wherein pathological conditions are associated with the unpaired expression of matrix-metalloproteases, wherein the pathological conditions are cardiovascular diseases, inflammatory diseases and malignant diseases.

28. The method of claim 27, comprising imaging active plaque burden.
